(19) United States
(12) Patent Application Publication
(10) Pub. No.: US 2022/0404340 A1
(43) Pub. Date: Dec. 22, 2022

(54) METHODS AND COMPOSITIONS FOR HIGH-THROUGHPUT COMPRESSED SCREENING FOR THERAPEUTICS

(71) Applicant: Massachusetts Institute of Technology, Cambridge, MA (US)

(72) Inventors: Benjamin Mead, Cambridge, MA (US); Conner Samuel Kummerlowe, Cambridge, MA (US); Alexander K. Shalek, Cambridge, MA (US)

(21) Appl. No.: 17/771,309

(22) PCT Filed: Oct. 23, 2020
(86) PCT No.: PCT/US2020/057131

§ 371 Date: Apr. 22, 2022

Related U.S. Application Data
(60) Provisional application No. 62/926,159, filed on Oct. 25, 2019.
(60) Provisional application No. 63/009,921, filed on Apr. 14, 2020.

(51) Int. Cl. G01N 33/50; G16B 5/00; G16B 40/20; G16C 20/50; G16H 20/10

(52) U.S. Cl. CPC G01N 33/5082; G01N 33/502; G16B 5/00; G16B 40/20; G16C 20/50; G16H 20/10

(57) ABSTRACT

Described in certain example embodiments herein are systems, methods, and uses thereof for high-throughput in vitro evaluating multiple test compounds in parallel for biological or pharmacological functions. In certain embodiments, the system allows the selection of a subset of test compounds from a group of test compounds to form an optimized pool, and methods are provided to use such optimized pool of test compounds to identify and validate therapeutic agents for treating diseases and driving guided differentiation of stem cells into desired types of cells. The systems described herein can provide, for example, a cost-effective and high-quality high-throughput approach for drug screening.

### CROSS-REFERENCE TO RELATED APPLICATIONS

This application claims the benefit of and priority to U.S. Provisional Patent Application Nos. 62/926,159, filed on Oct. 25, 2019, entitled “Methods and Compositions for High-Throughput Compressed Screening for Therapeutics”, and 63/009,921, filed on Apr. 14, 2020, entitled “Methods and Compositions for High-Throughput Compressed Screening for Therapeutics,” the contents of which are incorporated by reference herein in their entirety.

### STATEMENT REGARDING FEDERALLY SPONSORED RESEARCH

This invention was made with government support under Grants DE013023, HL095722, OD020839, AI089992, HL095791, CA217377, AI039671, AI118672, HG006193, CA202820, HL126554, DA046277, and AI138546 awarded by the National Institutes of Health and Graduate Research Fellowship Program from the U.S. National Science Foundation (NSF). The government has certain rights in the invention.

### TECHNICAL FIELD

The present invention is generally directed to methods, compositions, and uses thereof for in vitro high-throughput screening for therapeutics. The present invention also relates to methods of evaluating agents capable of guiding stem cell differentiation.

### BACKGROUND

The intestinal barrier plays critical roles in maintaining the structural integrity and physiological functions of intestine. A number of diseases arise from or result in the breakdown of intestinal barrier, including malignant diseases, infectious diseases, inflammatory disease, and autoimmune diseases. Compositions and methods for maintaining, repairing, and restoring the integrity and function of intestine may offer a unique therapeutic approach for the treatments of these diseases. Intestinal organoids, derived from intestinal stem cells (ISCs) and composed of ISCs, Paneth cells (PCs), enteroendocrine cells (EECs), goblet cells, and absorptive enterocytes have been invaluable to the study of intestinal biology. However, utilization of intestinal organoids for guided differentiation to produce desired types of cells and/or tissues that can be used in clinical applications remains unexplored. High-throughput methods and compositions are urgently needed for using intestinal organoids, and more generally organoids derived from diseased or healthy tissues and/or organs, to screen and identify effective therapeutics for treating diseases of the intestine and other organs.

Citation or identification of any document in this application is not an admission that such a document is available as prior art to the present invention.

### SUMMARY

In certain embodiments, methods and systems are provided for in vitro high-throughput screening for therapeutics using organoids or stem cells. In some embodiments, a system is disclosed that allows one to use computer-implemented method for selecting a subset of test compounds from a group of test compounds to form an optimized pool, and evaluating the pool of test compounds in parallel. Such optimized pool is characterized by low probability of interactions between molecules with known bioactivities and is approximated as an independent set of test compounds. In addition, the system provides a method for composing a plurality of such optimized pools from a group of test compounds. As a result, a given test compound can appear in different optimized pools to be evaluated in parallel with different subsets of test compounds, thus offering a versatile platform for deep and high-throughput screening of test compounds. Furthermore, the methods and systems disclosed herein allow for more compounds to be tested in a given number of biological samples, and for fewer biological samples to be used for testing a given number of compounds, thus providing a “compressed” approach for screening test compounds. The systems and methods provided herein are not only applicable to the screening of compounds disclosed in present invention, but also can be used for selecting a plurality of test compounds for any circumstances or any testing system.

In certain embodiments, methods of parallelly screening for therapeutic agents for treating a disease is provided. The method disclosed herein provides a particular advantage for situations where the availability of test subjects is limited, e.g. organoids derived from human biopsy. In some embodiments, the methods teach to use a plurality of test compounds in a pool optimized using the system disclosed herein for evaluating their biological activities in parallel on test subjects including cultured organoids or cells. In some embodiments, the organoids can be derived from stem cells from any tissues or organs.

In certain embodiments, methods and uses of screening for therapeutic agents for guiding differentiation of stem cells are provided. Using these methods, one can effectively identify agents capable of driving the differentiation of stem cells into a desired type of desired types of functional cells, thus being able to prepare an organoid enriched in the desired type or desired types of cells. In certain embodiments, the teachings also include how to compare the organoids prepared using methods disclosed herein with their in vivo tissue counterparts at molecular level and single-cell level, so that one can improve the process of making organoids to more faithfully mimic the in vivo tissue counterparts both structurally and functionally.

In certain embodiments, the methods and compositions disclosed herein can be used for identifying therapeutic agent for preparing certain types of cells required for maintaining the function and structural integrity of intestine, e.g. Paneth cells, enteroendocrine cells, and enterocytes. In some embodiments, the methods and compositions disclosed herein can also be applied in identifying therapeutic agents for preparing certain type of cells required for maintaining the function and structural integrity of other tissues and/or organs, e.g. the skin, eye, pancreas, brain, and liver.

One of the major advantages of this high-throughput screening method is that it allows a plurality of test compounds to be evaluated in parallel using a single unit of organoid culture or cell culture, e.g. a plurality of test compounds to be present simultaneously in one well of a culture plate, so that it greatly facilitates the efficiency of screening and/or identifying process by reducing substantially the time and cost, while removing possible errors introduced by using multiple organoids or cell cultures for testing multiple compounds.

Described in certain exemplary embodiments herein are computer-implemented methods for selecting a subset of test compounds from a group of test compounds to place in a pool of test compounds, wherein the pool of test compounds are to be evaluated in parallel for their biological functions, comprising, by one or more computing devices: receiving a request to evaluate a group of test compounds; determining chemical similarity of each test compound with every other test compound in the group of test compounds; determining a biological connectivity of each test compound with every other test compound in the group of test compounds, wherein the biological connectivity is assessed based on a transcriptional profile, mode of action, gene targets, effect on protein-protein interactions, or any combination thereof of each test compound; calculating an optimization score based in part on the chemical similarity and the biological connectivity of each test compound in the group; and based on the optimization score, assigning test compounds into the subset that can be evaluated together with minimal interaction or interference with other test compounds in a given subset.

In certain example embodiments, assigning test compounds into the subset is based on a gradient-free optimization algorithm.

In certain example embodiments, the method further comprises selecting a plurality of subsets such that each test compound is placed into at least one subset and a lowest total energy is determined for the plurality of subsets.

In certain example embodiments, the method further comprises receiving a number of test compounds to include in a plurality of subgroups of test compounds; determining the chemical similarity and the biological connectivity of each of the plurality of subgroups to determine the energy of each subgroup; and based on the determined energy for each subgroup, selecting subsets of test compounds that minimize the determined energy for each subgroup.

In certain example embodiments, the method further comprises calculating a scaled score of biological connectivity, a scaled score of chemical similarity, or both for each pair of test compounds evaluated, wherein the scaled scores of biological connectivity and chemical similarity are based on a scaling function; and based on the scaled score of biological connectivity, the scaled score of chemical similarity, or both for each pair of test compounds evaluated, selecting a subset of the group of test compounds to place in a pool for evaluation.

In certain example embodiments, the biological connectivity is based on a connectivity map of common gene expression signatures, and wherein the chemical similarity is based on the calculation of Tamonoto coefficient for each pair of test compounds.

In certain example embodiments, the pools of test compounds are optimized to test for combinatorial effects of agents versus isolation of individual agent effects.

Described in certain example embodiments herein are methods of parallel screening for therapeutic agents for treating a disease, the method comprising contacting a cell or a tissue with a pool of test compounds in parallel, wherein the test compounds in the pool are selected using the methods of any one of paragraphs [0011]-[0017]; evaluating the effect of the test compounds on the cell or tissue; and selecting one or more test compounds with a desired activity, whereby a plurality of test compounds are screened in parallel for therapeutic application in treating the disease.

In certain example embodiments, the tissue is an in vitro cultured organoid.

In certain example embodiments, the organoid is derived from stem cells originated from tissues or organs comprising the intestine, liver, pancreas, brain, ovary, uterus, skin, esophagus, lung, spleen, kidney, bone, bone marrow, brain, central nervous system, peripheral nervous system, blood, cartilage, fat, and eye.

In certain example embodiments, the number of test compounds in a pool is any number between 1 and 1,000,000,000,000.

In certain example embodiments, the effect of the test compounds is evaluated by measuring changes in biological activities comprising transcriptomics, genomics, epigenomics, proteomics, genetics, epigenetics, metabolomics, multiomics, phenotype, or any combination thereof.

In certain example embodiments, the effect of the test compounds is evaluated by measuring changes in transcriptomics.

In certain example embodiments, the measurement of the effect of the test compounds is performed at single-cell level.

Described in certain example embodiments herein are methods of screening for therapeutic agents for guiding cell differentiation, the method comprising contacting an in vitro cultured organoid or a cultured cell with a pool of test compounds in parallel, wherein the test compounds in the pool are selected using the methods of any one paragraphs [0011]-[0017]; evaluating the effect of test compounds on cell differentiation of the organoid or cultured cell; and selecting the one or more test compounds capable of guiding the differentiation of the organoid or the cultured cell from a first cell state to a second cell state.

In certain example embodiments, the cultured cell is a stem cell.

In certain example embodiments, the organoid is derived from stem cells that originated from, were isolated from, or were derived from tissues or organs comprising the intestine, liver, pancreas, brain, ovary, uterus, skin, esophagus, lung, spleen, kidney, bone, bone marrow, brain, central nervous system, peripheral nervous system, blood, cartilage, fat, and eye.

In certain example embodiments, the cultured cell is isolated from tissues or organs comprising the intestine, liver, pancreas, brain, ovary, uterus, skin, esophagus, lung, spleen, kidney, bone, bone marrow, brain, central nervous system, peripheral nervous system, blood, cartilage, fat, and eye.

In certain example embodiments, the number of test compounds in a pool is any number between 1 and 1,000,000.

In certain example embodiments, the differentiation of the organoid or cultured cell is measured by changes in comprising phenotype, genotype, genomics, epigenomics, transcriptomics, genetics, epigenetics, proteomics, multiomics, metabolomics, or any combination thereof.

In certain example embodiments, the measurement is performed at single-cell level.

In certain example embodiments, the second cell state is a differentiated, functional cell selected from the group consisting of: a Paneth cell, goblet cell, M cell, Tuft cell, enteroendocrine cell, enterocytes, hepatocyte, pancreatic B-cell, pancreatic alpha-cell, neuron, glia cell, brain cell, keratinocyte, melanocyte, epithelial cell, endothelial cell, hematopoietic cell, T lymphocyte, B lymphocyte, natural killer cell, dendritic cell, macrophage, monocyte, neutrophil, eosinophil, basophil, megakaryocyte, platelet, adipocyte, osteoblast, osteoclast, chondrocyte, and a combination thereof.

Described in certain example embodiments herein are methods for determining specific biological effect, pharmacological effect, or both of a test compound in a test pool, comprising forming an optimized pool containing the test compound of interest according to any of paragraphs [0011]-[0017]; testing the biological and/or pharmacological effects of the pool of testing compounds according to any of the preceding claims; and performing deconvolution of the biological effects, pharmacological effects, or both of the pool, wherein the deconvolution comprises computational de-coding using methods comprising large linear computational models, whereby the specific biological effect, pharmaceutical effect, or both of a test compound are determined.

Described in certain example embodiments herein are methods of determining one or more characteristics of a disease, comprising: contacting a biological sample in vitro with a pool of test compounds in parallel, wherein the test compounds in the pool are selected using the methods of any one of paragraphs [0011]-[0017]; evaluating the effect of one or more of the test compounds in the pool of test compounds on the biological sample, whereby the specific biological effect, pharmaceutical effect, or both of a test compound are determined and whereby the biological effect, pharmaceutical effect, or both of a test compound determined is/are indicative of one or more disease characteristics.

In certain example embodiments, the one or more disease characteristics is evaluated by measuring or evaluating one or more of the following: expression, activity, or function of one or more genes, proteins, gene programs, biological pathways, cell processes, cell or tissue functions, or combinations thereof in the biological sample.

In certain example embodiments, evaluating the effect of one or more of the test compounds comprises measuring changes in one or more biologic activities of the biological sample.

In certain example embodiments, measuring changes in one or more biologic activities comprises a transcript or transcriptome analysis, a gene or genomic analysis, an epigenome analysis, a protein or proteome analysis, a metabolomic analysis, a multiomic analysis, a phenotype analysis, a genetic analysis, or a combination thereof.

In certain example embodiments, evaluating the effect of one or more of the test compounds further comprises performing deconvolution of the biological effects, pharmacological effects, or both the pool, wherein the deconvolution comprises computational de-coding using one or more methods comprising large linear computational models.

In certain example embodiments, the biological sample is a cell or cell population, organoid, or tissue.

In certain example embodiments, the biological sample is a biopsy sample obtained from a subject.

In certain example embodiments, the biological sample is isolated from, derived from, or comprises, cells or tissue of the intestine, liver, pancreas, ovary, uterus, skin, esophagus, lung, spleen, kidney, bone, bone marrow, brain, central nervous system, peripheral nervous system, blood, cartilage, fat, eye, heart, lymph node, lymphatic system, thyroid, endocrine system tissue or gland, or a combination thereof.

In certain example embodiments, the test pool is an optimized test pool.

In certain example embodiments, the number of test compounds in the test pool is any number from 1 to 1,000,000.

Described in certain example embodiments herein are methods of diagnosing, prognosing, monitoring, or staging a disease in a subject, comprising contacting, in vitro, a biological sample obtained from the subject with a pool of test compounds in parallel, wherein the test compounds in the pool are selected using the methods of any one of paragraphs [0011]-[0017]; evaluating the effect of one or more of the test compounds in the pool of test compounds on the biological sample, whereby the specific biological effects, pharmaceutical effects, or both are determined and whereby the specific biological effects, pharmaceutical effects, or both of a test compound determined is/are indicative of a disease, a disease symptom, and/or stage of a disease in the subject.

In certain example embodiments, evaluating the effect of one or more of the test compounds comprises measuring changes in one or more biologic activities of the biological sample.

In certain example embodiments, measuring changes in one or more biologic activities comprises a transcript or transcriptome analysis, a gene or genomic analysis, an epigenome analysis, a protein or proteome analysis, a multiomic analysis, a metabolomic analysis, a phenotype analysis, a genetic analysis, or a combination thereof.

In certain example embodiments, evaluating the effect of one or more of the test compounds further comprises performing deconvolution of the biological effects, pharmacological effects, or both of the pool, wherein the deconvolution comprises computational de-coding using one or more methods comprising large linear computational models.

In certain example embodiments, the biological sample is a cell or cell population, organoid, or tissue.

In certain example embodiments, the biological sample is a biopsy sample obtained from a subject.

In certain example embodiments, the biological sample is isolated from, derived from, or comprises cells or tissue of the intestine, liver, pancreas, ovary, uterus, skin, esophagus, lung, spleen, kidney, bone, bone marrow, brain, central nervous system, peripheral nervous system, blood, cartilage, fat, eye, heart, lymph node, lymphatic system, thyroid, endocrine system tissue or gland, or a combination thereof.

In certain example embodiments, the test pool is an optimized test pool.

In certain example embodiments, the number of test compounds in the test pool is any number from 1 to 1,000,000.

Described in certain example embodiments herein are pharmaceutical formulations comprising one or more active agents; and a pharmaceutically acceptable carrier, wherein the one or more active agents is identified as an active agent by performing a method as in any one of paragraphs [0018]-[0043].

Described in certain example embodiments herein are methods of guiding differentiation of a cell from a first cell state to a second cell state comprising contacting a cell or cell population with one or more compounds capable of guiding differentiation of a cell, wherein the one or more active agents is identified as an active agent by performing a method as in any one of paragraphs [0025]-[0032].

In certain example embodiments, the cell or cell population is a stem cell or stem cell population.

Described in certain example embodiments herein are differentiated cells, cell populations, tissues, or organoids, wherein the differentiated cells, cell populations, tissues, or organoids are produced by a method of guided differentiation as in any one of paragraphs [0054]-[0055].

Described in certain example embodiments herein are methods of treating a subject in need thereof, comprising administering a pharmaceutical formulation as in paragraph [0053], a differentiated cell, cell population, tissue, or organoid as in paragraph [0056], or both to the subject in need thereof.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—A schematic of general structure of the system used for selecting a plurality of test compounds to form a pool in which the test compounds have a minimal scaled score of biological connectivity.

FIG. 2—A schematic flow diagram that shows the steps used by the computer system in selecting a plurality of test compounds to form a pool in which the test compounds have a minimal scaled score of biological connectivity.

FIG. 3—A schematic of the computing system used for selecting a plurality of test compounds to form a pool in which the test compounds have a minimal scaled score of biological connectivity.

FIGS. 4A-4B—Energy (cost) per test compound in a pool is substantially reduced after the optimization of biological connectivity. FIG. 4A shows the start cost before optimization and FIG. 4B shows the final cost after optimization. Numbers by each plot point indicate number of drugs per pool. Number of replicates are indicated below each plot line.

FIGS. 5A-5B—Chemical similarity among test compounds in a pool is not substantially changed after optimization. Each individual graph in FIG. 5A shows the before (gray bars) and after (black bars) pool optimization with each showing mean similarity per pool (x-axis) versus frequency (y-axis). The x-axis values range from 0 to 2.5 and y-axis values range from 0 to 1200 in each individual graph of FIG. 5A. FIG. 5B shows KS distance between mean connectivities per pool before and after optimization.

FIGS. 6A-6B—Biological connectivity among test compounds in a pool is substantially reduced after optimization. Each individual graph in FIG. 6A shows the before (gray bars) and after (black bars) pool optimization with each showing mean connectivity per pool (x-axis) versus frequency (y-axis) for each combination of pool size and replicates noted. The x-axis values range from 0 to 5.0 and y-axis values range from 0 to 1000 in each individual graph of FIG. 6A. FIG. 6B shows KS distance between mean connectivities per pool before and after optimization.

FIG. 7—Repeated drug paring in a pool is rare. It increases primarily with the increase in pool size, i.e. the number of test compounds per pool. This can imply that as designed that it is not the best way to assess drug interactions, while the right model and high enough replicates is likely a good way to measure gene-drug effects. Each individual graph shows the number of times a pair occurs together (x-axis) versus the frequency (y-axis) for each combination of pool size and replicates noted. The x-axis values range from 0 to 5 and y-axis values range from 0 to 10⁶.

FIG. 8—Compressed screening described in embodiments herein can, among other things, reduce the amount of biomass needed for effective screening. The top graphic demonstrates the number of samples needed without compressed screening via one or more embodiments described herein. The bottom graphic can demonstrate the reduction of biomass needed for screening when using one or more of the several embodiments of compressed screening described herein.

FIG. 9—Overview of using computational methods to facilitate the rational design and/or optimization of pools for embodiments of compressed screening described to maximize perturbations while minimizing the amount or biomass needed.

FIG. 10—Compressed screening as described herein can unlock models and readouts and allow for, among other things, rich data sets from minimized biomass of complex samples.

FIG. 11—Optimization independent pools coupled with decoding pooled screens can, among other things, reduce the number or replicates per drug needed for an effective screen. In some embodiments, a matrix of drug-drug similarity can be used to calculate the ‘cost’ or ‘energy’ of any given pool and optimize for minimally interacting pools in bio-active libraries, which can provide for many-fold compression.

FIG. 12—A general linear model for deconvolution of pooled results to determine individual agent (e.g. compound, drug, or other potentially active agent) effect.

FIG. 13—Diagram of exemplary dataset for a compressed screening assay.

FIG. 14—Design of compressed screening of human intestinal organoids.

FIG. 15—First pass analysis of a simple readout from the screen, in the case of the human intestinal organoids experiment: cell morphology. All images were observed and were classified into one of four different classes: “branchy”, “dead”, “small”, and “cystic”.

FIG. 16—Results from the simple readout demonstrating that three drugs are lethal, which were identified in 27 wells in a conventional randomized screen and 30 wells of an optimized screen described by one or more embodiments herein.

FIG. 17—Optimization of the rational design of pools in the compressed screening can reduce the redundancy in samples needed to demonstrate an effect of an agent in a screen.

FIG. 18—Some phenotypes do not need optimization to discern agent effect(s). For example, in human intestinal organoids that displayed a “small” or “cystic” phenotype as determined during a first pass analysis (see e.g. FIG. 15) both randomized and optimized pools identified one agent driving each phenotype (FGFR inhibitor in the case of the “small” phenotype and prostanoid receptor agonist in the case of the “cystic” phenotype). In optimized and randomized settings, one drug is clearly driving each phenotype.

FIG. 19—Some phenotypes benefit from optimization to discern agent effect(s). For example, in human intestinal organoids that displayed a “branched” phenotype optimization provided clearer insight into agents affecting the phenotype.

FIG. 20—Effects of some agents can be buried or “swamped out” using conventional randomized screening techniques. For example, in the case of the human intestinal organoids the effect of BRD-K64052570 (see e.g. FIG. 19) could have been lost without optimization, particularly in the branched phenotype. This loss can be attributed to different agents having similar effects. For FIG. 20, randomization and optimization were rerun 10 times. Overlaps between BRD-K64052570 and the five other effective drugs. As shown in FIG. 20 BRD-K64052570 overlapped with other effective drugs and optimization reduced the overlap with these drugs and allow the effect to be discerned.

FIGS. 21A-21B—Effective agents can be connected by CMAP scores. In the case of the human intestinal organoids the effective agents were connected by high CMAP scores.

FIG. 22—Shows clustering CMAP scores for all 127 agents tested against the human intestinal organoids. FIG. 22 can demonstrate that some drugs with similar effects clustered by phenotype based on effect. For example, 3 lethal drugs clustered with the “small” and “branchy” phenotype. Drugs 1-127 are listed in Table 1.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2nd edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011)

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murine, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader embodiments discussed herein. One embodiment described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### OVERVIEW

Embodiments disclosed herein provide methods, compositions, and uses thereof for high-throughput in vitro screening of a plurality of test compounds in parallel for therapeutic or other uses. In some embodiments, a system is disclosed for selecting a subset of test compounds from a group of test compounds to form an optimized pool. Such formed pool is characterized by having minimal biological connectivity among the test compounds within the pool. Therefore, with multiple replicates for a given test compound appearing in different pools, the biological function of each test compound can be deciphered from a plurality of test compounds. In other words, the pools are optimized such that compounds in any given pool have a low probability of interacting such that a pool can be approximated as an independent set of tests, thus increasing the efficiency of the pools and screening methods described and demonstrated herein. To demonstrate the method, a plurality of test compounds was evaluated for their ability to guide differentiation of ISCs to desired types of cells in an organoid, which is set forth in the Working Examples herein.

In some embodiments, methods are disclosed to use the system disclosed herein for forming a pool containing a plurality of test compounds for screening therapeutic agents for treating a disease or diseases. In some embodiments, methods disclosed herein are used for screening therapeutic agents for guiding differentiation of stem cells into desired type or types of cells. In some embodiments, methods disclosed herein are used for screening therapeutic agents for guiding differentiation of intestinal stem cells (ISC) into functional cells including, but not limited to, Paneth cells, M cells, Tuft cells, enteroendocrine cells, and enterocytes. One of the major advantages of the present invention is that multiple test compounds can be evaluated simultaneously for their biological and pharmaceutical activities using a single testing system, e.g. an organoid in a well of a culture plate. As a result, the time and cost for drug screening can be substantially reduced, while the quality of drug screening can be substantially increased due to the removal of possible errors occurring during singleplex screening in which multiple test subjects are used for multiple test compounds. Furthermore, by testing compounds in parallel in a pool, the present invention provides great benefits for situations in which the availability of test subjects is limited, e.g. biopsy specimens from patients or other difficult-to-obtain sources.

In some embodiments, methods for making an organoid that is suitable for high-throughput in vitro screening of test compounds is also disclosed. These methods teach the optimization of culture conditions for proper preparation of organoids from animal or human biopsy specimens. Such formed organoids can be used for testing the guided differentiation of stem cells into desired type or desired types of functional cells.

In some embodiments, methods for evaluating the effectiveness of an organoid for in vitro screening of test compounds are disclosed. These methods comprise massively-parallel bulk population DNA and RNA sequencing, massively-parallel single-cell RNA sequencing, phenotype determination, cellular imaging, molecular imaging, spatial-molecular imaging, spatial transcriptomics, flow cytometry, soluble assays, and reporter cell lines. One or more of these methods can be used to evaluate the gene expression program at single cell level in an organoid growing from stem cells derived from animal or human biopsy specimens and the source tissues as well. The gene expression programs of single cells in an organoid can faithfully reflect how similarly it resembles the source tissue in vivo from which it was derived. One of the major advantages of these methods is that one can improve the conditions under that an organoid is prepared so that the resulting organoid can faithfully mimic the in vivo source tissue.

In some embodiments, methods for identifying therapeutic targets for a disease are disclosed. The therapeutic targets identified using the methods provided herein can more faithfully represent authentic changes in molecular machinery and the cell state, thus providing attractive modality for screening and evaluating drugs that are capable of treating the disease through acting on the targets.

### Systems and Methods for Selecting Pools of Test Compounds for Parallel Screening

Described in exemplary embodiments herein are systems and methods for selecting and/or optimizing pools of test compounds for parallel screening and parallel screening of said pools. In some embodiments, the systems and methods can provide for selection of subsets of test compounds to form pools and/or optimized pools of test compounds for parallel screening. Described in exemplary embodiments herein are pools that can contain a plurality of test compounds and are also referred to herein as “test pools”. As used herein, the term “pool” refers to a plurality of agents (for example, test compounds) that are grouped together. In some embodiments, the test pool can contain a plurality of test compounds that can be selected such that an optimized pool of test compounds is formed where the biological connectivity of the test compounds is optimized and minimized. This can result in a minimized average scaled score for the biological connectivities among the test compounds in the pool. A scaled biological connectivity score can be calculated for each measured metric. In some embodiments, provided are system, methods, and uses thereof for selecting a plurality of test compounds to form a pool so that the biological connectivity of the plurality of test compounds in such a pool is optimized and minimized. “Biological connectivity” as the term is used herein, refers to how similar two or more compounds (or other agents) are based upon their biological function and/or activity in a given context (such as cell type, cell state, disease state, etc.). Biological connectivity can be presented in the form of a biological connectivity map, which is further defined and discussed elsewhere herein. Biological connectivity can be evaluated by measuring an effect or response of a subject to each compound and comparing the effect(s) or response. Biological connectivity can be evaluated by measuring and/or considering, for example, one or more signatures (e.g. gene, protein, or other signatures) of the subject; one or more gene, transcript, protein, epigenome, metabolome, secretome, function, activity or interaction; mode of action of each of the one or more compounds; one or more gene targets, transcript targets, protein targets, epigenome targets, or a combination thereof of each compound; each compound's effect on protein-protein interactions and/or gene-protein interactions; and any combination thereof. As used in this context herein, “target” refers to a gene, transcript, protein, and/or epigenome or region there of that is specifically acted upon or associates with a particular compound said to target that gene, transcript, protein and/or epigenome or region thereof.

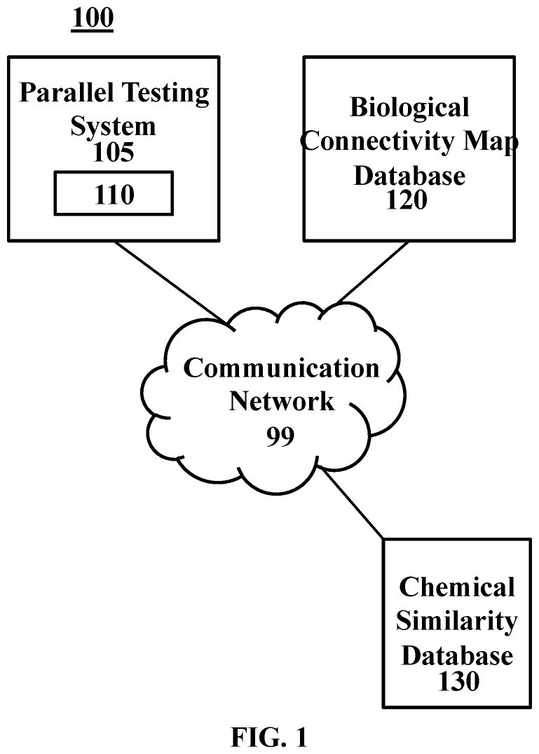

In some embodiments, a system is disclosed for performing the selection work as depicted in, e.g. FIG. 1. This system comprises, in some embodiments, a parallel testing system, a communications network, a biological connectivity map database, and a chemical similarity database. The system can be configured to perform one or more of the methods described herein, including, but not limited to, selecting test compounds for pools and/or optimizing pools of test compounds. These and other methods are described elsewhere herein. As used herein, the term “computer-implemented” refers to performing a method, executing processing logic stored in memory, or other action via a computing device. Computing devices are any device, machine, and/or system (electronic or otherwise) capable of taking inputs in any suitable form, processing the inputs according to one or more processing logics (e.g. capable of executing processing logic), and providing an output based on results from processing. Such processing can be executed automatically based upon the receiving an input. Inputs can be provided manually or automatically. Computing devices can be fixed or mobile. Computing devices can be connected physically, wirelessly, electronically, so as to form a network.

In some embodiments, the parallel testing system is an in vitro system or an in vivo system. In some embodiments, the parallel testing system comprises test subjects comprising organoids, stem cells, primary tissues, in vitro organ cultures, primary cell cultures, immortalized cell cultures, and cultured cell lines. In some embodiments, the test subjects in the parallel testing system are contained or cultured in a cell/tissue culture vessel comprising tissue culture plates, culture flasks, and other type of in vitro culture vessels. In some embodiments, well defined culture medium is used for maintaining the test subjects. In some embodiments, a specifically formulated culture medium is used for maintaining the test subjects.

In some embodiments, a test subject is defined as a subcellular organelle, a cell, an organoid, a tissue, an organ, a physiological system of animal or human, or the whole body of an animal or human. In some embodiments, an in vitro or ex vivo test subject is used. In some embodiments, an in vivo test subject is used. In some embodiments, an in vivo animal test subject is used. In some embodiments, an in vivo human test subject is used. In some embodiments, an ex vivo organ test subject from an animal or a human is used.

In some embodiments, the parallel testing system comprising contacting a test subject with a plurality of test compounds in an optimized pool. In some embodiments, a phenotype of the subject is measured. In some embodiments, a genotype of the subject is measured. In some embodiments, the growth of test subject is measured. In some embodiments, the differentiation of test subject is measured. In some embodiments, the cell death of test subject is measured. In some embodiments, the death of test subject is recorded. In some embodiments, biological functions and activities comprising cell-cell interactions, cell adhesion, cell mobility, and endocytosis are measured.

In some embodiments, a biological connectivity map is defined as a large-scale compendium of functional perturbations in cultured human cells or tissues or organoids coupled to a functional readout, phenotype readout, biological activity readout, and/or gene-expression readout, so that genes, functional molecules, biological activities, therapeutic agents, physiological status, and/or pathological status are connected. A biological connectivity map database facilitates the discovery of connections between genes, drugs, and diseases. In some embodiments, a biological connectivity map database is similar to the one as previously shown (see e.g., Subramanian et al., 2017, Cell 171, 1437-1452).

In some embodiments, the system disclosed herein comprises a biological connectivity map database. In some embodiments, the biological connectivity map database comprises biological activity of a plurality of compounds comprising therapeutic agents, large molecules, antibodies, vaccines, and small molecules. In some embodiments, the biological connectivity map database comprises the changes in structure, activity, and function of genes, proteins, receptors, and other functional biomolecules. In some embodiments, the biological connectivity map database comprises the physiological and pathological states of an animal or a human. In some embodiments, the metrics of the biological connectivity map database are all connected to each other.

In some embodiments, the system disclosed herein further comprises a chemical similarity database. As used herein, “chemical similarity” is defined as the similarity of chemical elements, molecules or chemical compounds with respect to either structural or functional qualities, i.e. the effect that the chemical compound has on reaction partners in inorganic or biological settings. Biological effects and thus also similarity of effects are usually quantified using the biological activity of a compound. In general terms, function can be related to the chemical activity of compounds (among others). In some embodiments, compounds with high chemical similarity have similar biological functions. In some embodiments, compounds with high chemical similarity have different biological functions. The most popular similarity measure for comparing chemical structures represented by means of fingerprints is the Tanimoto (or Jaccard) coefficient T. Two structures are usually considered similar if T>0.85. However, it is a common misunderstanding that a similarity of T>0.85 reflects similar bioactivities in general.

In some embodiments, the system disclosed herein calculates the chemical similarity based on Tanimoto (or Jaccard) coefficient (T) method or a modified version of Tanimoto (or Jaccard) method. In some embodiments, the system disclosed herein utilizes a database available to the system. In some embodiments, test compounds with similarity metric T>0.85 are precluded to be included in the same given pool.

In some embodiments, the system disclosed herein comprises a communication network that connects the components together. The components comprise a parallel testing system, a biological connectivity map database, and a chemical similarity database. In some embodiments, the system disclosed herein functions as a whole. Described in certain example embodiments herein are computer-implemented methods for selecting a subset of test compounds from a group of test compounds to place in a pool of test compounds, wherein the pool of test compounds are to be evaluated in parallel for their biological functions, comprising, by one or more computing devices: receiving a request to evaluate a group of test compounds determining chemical similarity of each test compound with every other test compound in the group of test compounds; determining a biological connectivity of each test compound with every other test compound in the group of test compounds, wherein the biological connectivity is assessed based on a transcriptional profile, mode of action, gene targets, effect on protein-protein interactions, or any combination thereof of each test compound; calculating an optimization score based in part on the chemical similarity and the biological connectivity of each test compound in the group; and based on the optimization score, assigning test compounds into the subset that can be evaluated together with minimal interaction or interference with other test compounds in a given subset.

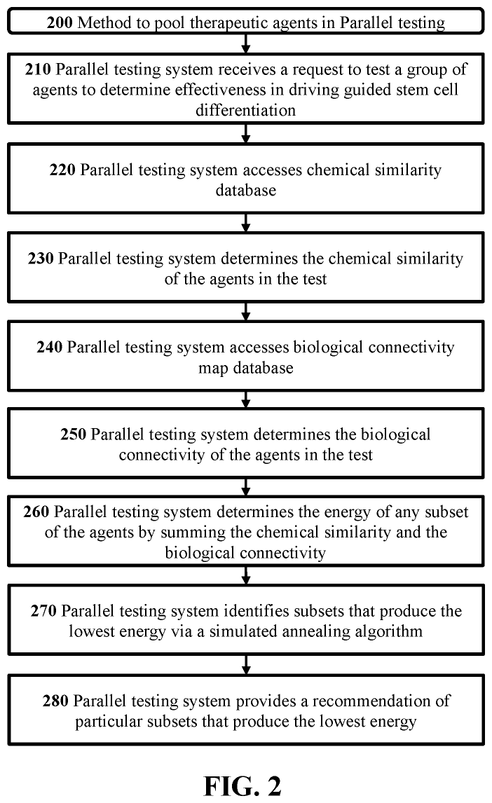

In some embodiments, a method to pool test compounds in parallel testing 200 is disclosed herein as depicted in FIG. 2. The method comprises steps for performing the task of selection. In some embodiments, the method comprises a step 210 that the parallel testing system receives a request to test a group of agents to determine effectiveness in driving guided stem cell differentiation or in other biological and/or pharmacological activities.

In some embodiments, chemical similarity and biological connectivity scores are combined to form an optimization score via the following method. Originally biological connectivity scores range from −100 to 100. The absolute value of these scores (ranging from 0 to 100) are used as in the method a negative correlation is equally informative as a positive correlation. The score is then scaled to the 0 to 1 range such that the score now has the same range as the chemical similarity.

Both the chemical similarity and biological connectivity scores are then scaled using Formula 1:

$$y = \frac{1}{( 1 + {e}^{- 20 x + 15} )} . \qquad ( Formula 1 )$$

This can also be referred to herein as a scaling function.

In applying this function (or a number of other similar functions), drug pairs with chemical similarity or connectivity less than 0.75 are down-weighted and drug pairs with higher values are up-weighted. This allows the optimization to focus on pairs that have particularly high values, which makes the optimization problem more tractable.

Subsets that find the lowest energy are identified 270 by using, for example, a simulated annealing algorithm or other suitable algorithm. This simulated annealing algorithm works by randomly initializing the drugs into pools and then randomly swapping the drug pairs. At each random swap, the energy favorability of the swap is calculated. If the swap is favorable, the swap is accepted. If it is not favorable, the swap is accepted with probably k1−e^{ΔE/k}^{_{B}}^{T} (Formula 2), where ΔE is the energy difference between the system before and after the swap, k_{B} is the Boltzmann constant (a known physical parameter) and T is a temperature set by an empirically determined cooling schedule. At the start of the optimization, T is high such that the system can explore many states, while at the end of the optimization T is low such that the system can settle into a low energy state. One of the characteristics and functionalities of this algorithm comprises that it allows for an efficient search of the energy space and is much faster than looking at all possible combinations.

In some embodiments the parallel testing system provides a recommendation of particular subsets of test compounds that produce the lowest energy 280, which can be used as, for example an optimized pool.

In some embodiments, the method comprises a step 220 that the parallel testing system accesses chemical similarity database. In some embodiments, the chemical similarity database comprises calculated T metrics for each test compound. In some embodiments, the T metric is compared among the test compounds.

In some embodiments, the method comprises a step 230 that the parallel testing system determines the chemical similarity of the test compounds in the group. In some embodiments, test compounds with T>0.85 are precluded from being staying in a given pool.

In some embodiments, the method comprises accessing biological connectivity map database by the parallel testing system 240. In some embodiments, the biological connectivity map database comprises measurements in transcriptome, genome, epigenome, genetics, epigenetics, proteome, metabolome, or any combination thereof. In some embodiments, the biological connectivity map is composed with a method similar to previous reported CMap (see e.g., Subramanian et al., 2017, Cell 171, 1437-1452). In some embodiments, the biological connectivity map is composed with revised or different methods as the reported CMap. In some embodiments, the biological connectivity used herein comprises more than the CMap.

In some embodiments of the method, the parallel testing system determines 250 the biological connectivity of the test compounds in the group. In some embodiments of the method, the parallel testing system determines the energy (cost) of a given subset of test compounds by summing the chemical similarity and the biological connectivity. In some embodiments, the energy (cost) per test compound in a given subset of test compounds is calculated using models comprising linear model or non-linear model. In some embodiments, the parallel testing system identifies a subset or a plurality of subsets of test compounds that produce the lowest energy (cost) per test compound using a gradient-free optimization algorithm, and parallel testing system providing a recommendation of particular subsets that produce the lowest energy. In some embodiments, the gradient-free optimization algorithm has some modifications to fit into the purpose of the present invention. In some embodiments, the algorithm used for optimization is simulated annealing. In some embodiments, simulated annealing is an adapted Metropolis-Hastings algorithm. By annealing, the probability of accepting non-optimal energy is lower with consecutive test compound swaps. Gradient-free optimization is also referred to in the art as derivative free optimization and generally refers to algorithms that proceed without going through the process of calculating derivatives (e.g. first, second, etc. derivatives). Such processes and algorithms will be appreciated by those of skill in the art, any of which can be modified and adapted for use with the present invention in view of the descriptions provided herein.

In some embodiments, the method can include receiving a number of test compounds to include in a plurality of subgroups of test compounds, determining the chemical similarity and the biological connectivity of each of the plurality of subgroups to determine the energy of each subgroup, and based on the determined energy for each subgroup, selecting subsets of test compounds that minimize the determined energy for each subgroup. Minimized energy between subgroups can be determined as previously described with respect to selecting subsets of target compounds to include in pools having minimal or minimized energy.

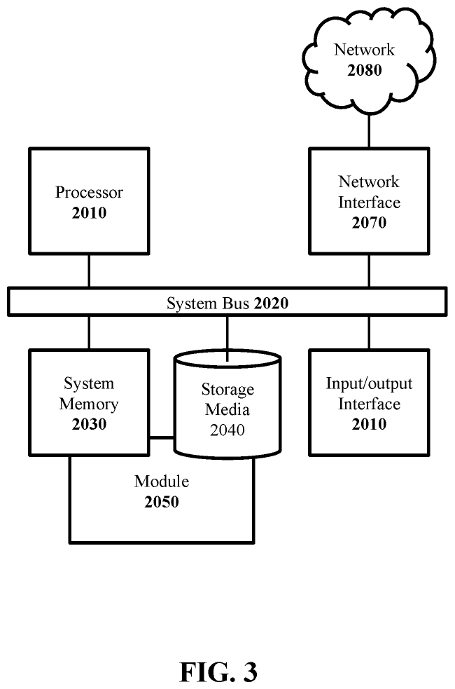

In some embodiments, a computing system is disclosed herein for implementing the process of selecting a plurality of test compounds for a pool with minimal average biological connectivity as depicted in FIG. 3. The system comprises a system bus, a processor, a network interface that is connected to a network, a system memory, a storage media, and an input/output interface. The storage media and system memory together form a module.

In some embodiments, the number of test compounds to be placed in a given pool is pre-determined. In some embodiments, the number of test compounds to be placed in a given pool is not pre-determined and requires to be adjusted empirically. In some embodiments, the number of replicates for a given test compound to appear in different pools is pre-determined. In some embodiments, the number of replicates for a given test compound to appear in different pools is not pre-set and requires to be adjusted empirically.

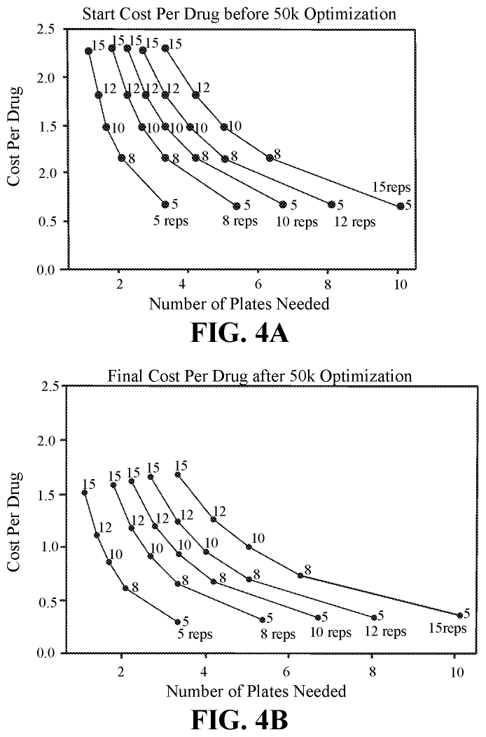

In some embodiments, the optimization steps take a number of iterations. In some embodiments, the number of iterations is greater than 10, greater than 100, greater than 10,000, greater than 1 million, greater than 10 million, greater than 100 million, greater than 1 billion, greater than 100 billion, greater than 1 trillion, or greater than 100 trillion. In some embodiments, the number of iterations is between 1,000 and 1 million. In some embodiments, the number of iterations is between 10,000 and 100,000. In some embodiments, the number of iterations is 50,000. After the number of iterations, an optimized pool is produced using the methods and system disclosed herein as depicted in FIGS. 4A-4B. Such an optimized pool has substantially reduced energy/cost per test compound.

In some embodiments, as selected using such optimization process, the test compounds in a given pool are “independent” to each other with none or minimal biological connectivity among them.

In some embodiments, optimized pools contain sets of test compounds having minimal or minimized energy, which are produced by swapping one test compound in the pool per step of optimization (or iteration).

In some embodiments, the number of test compounds to be optimized for pooling can be any number between 2 and 1,000,000. In some embodiments, the number of test compounds to be optimized for pooling can be more than 1,000,000. In some embodiments, the number of test compounds in an optimized pool can be any number between 2 and 1,000,000. In some embodiments, the number of test compounds in an optimized pool can be more than 1,000,000. In some embodiments, the number of test compounds in an optimized pool comprises 2, 3, 4, 5, 6, 7, 8, 9, 10, 11, 12, 13, 14, 15, 16, 17, 18, 19, or 20. In some embodiments, the number of test compounds in an optimized pool is more than 20, more than 30, more than 40, more than 50, or more than 100.

In some embodiments, a plurality of such optimized pools are produced for screening of a group of test compounds in parallel. In some embodiments, a given test compound can appear in different optimized pool. The number of times a given test compound appears in different distinct pools is called replicate. In some embodiments, the number of replicates for a test compound comprises 1, 2, 3, 4, 5, 6, 7, 8, 9, 10, 11, 12, 13, 14, 15, 16, 17, 18, 19, or 20. In some embodiments, the number of replicates for a test compound is more than 20, more than 30, more than 40, more than 50, more than 100, or more than 500.

In some embodiments, the optimization leads to a reduction in energy (cost) per test compound by 10%, 20%, 30%, 40%, 50%, 60%, 70%, 80%, or 90%.

In some embodiments, the optimization comprises the optimization of both of the number of test compounds in a pool and the number of replicates of each test compound.

### Test Compounds

As used herein, “test compound” refers to any compound, composition, cell, molecule, element, that can be rationally or randomly chosen to use in a method, such as a screening method described elsewhere herein to determine an effect it may cause alone or in combination with one or more other compounds on a biological sample. One of ordinary skill in the art will be able to, in view of this disclosure, rationally or randomly identify and choose, compounds to be used as test compounds within the screening methods herein. Rational test compound choices can be made based upon, inter alia, the interests, objectives, diseases to be investigated or treated, biological sample type, or other criteria, that will be appreciated by those of ordinary skill in the art in view of this disclosure. Random identification and choice of test compounds can be achieved by, for example, using an array of compounds without deference to any particular input or desired application or output.

Test compounds can be biologic or non-biologic molecules, metals, non-metals, metalloids, natural compounds, synthetic compounds, radioactive compounds, non-radioactive compounds, optically active compounds, non-optically active compounds, small molecules, large molecules, and combinations thereof. Test compounds can be without limitation, DNA, RNA, amino acids, peptides, polypeptides, antibodies, aptamers, ribozymes, guide sequences for ribozymes that inhibit translation or transcription of essential tumor proteins and genes, gene modification compounds, compositions, or complexes, hormones, immunomodulators, antipyretics, anxiolytics, antipsychotics, analgesics, antispasmodics, anti-inflammatories, anti-histamines, anti-infectives, radiation sensitizers, chemotherapeutics.

Exemplary hormones include, but are not limited to, amino-acid derived hormones (e.g. melatonin and thyroxine), small peptide hormones and protein hormones (e.g. thyrotropin-releasing hormone, vasopressin, insulin, growth hormone, luteinizing hormone, follicle-stimulating hormone, and thyroid-stimulating hormone), eicosanoids (e.g. arachidonic acid, lipoxins, and prostaglandins), and steroid hormones (e.g. estradiol, testosterone, tetrahydro testosterone, cortisol).

Exemplary immunomodulators include, but are not limited to, prednisone, azathioprine, 6-MP, cyclosporine, tacrolimus, methotrexate, interleukins (e.g. IL-2, IL-7, and IL-12), cytokines (e.g. interferons (e.g. IFN-α, IFN-β, IFN-ε, IFN-K, IFN-ω, and IFN-γ), granulocyte colony-stimulating factor, and imiquimod), chemokines (e.g. CCL3, CCL26 and CXCL7), cytosine phosphate-guanosine, oligodeoxynucleotides, glucans, antibodies, and aptamers).

Suitable antipyretics include, but are not limited to, non-steroidal anti-inflammants (e.g. ibuprofen, naproxen, ketoprofen, and nimesulide), aspirin and related salicylates (e.g. choline salicylate, magnesium salicylate, and sodium salicylate), paracetamol/acetaminophen, metamizole, nabumetone, phenazone, and quinine.

Exemplary anxiolytics include, but are not limited to, benzodiazepines (e.g. alprazolam, bromazepam, chlordiazepoxide, clonazepam, clorazepate, diazepam, flurazepam, lorazepam, oxazepam, temazepam, triazolam, and tofisopam), serotenergic antidepressants (e.g. selective serotonin reuptake inhibitors, tricyclic antidepressants, and monoamine oxidase inhibitors), mebicar, afobazole, selank, bromantane, emoxypine, azapirones, barbiturates, hydroxyzine, pregabalin, validol, and beta blockers.

Exemplary antipsychotics include, but are not limited to, benperidol, bromoperidol, droperidol, haloperidol, moperone, pipaperone, timiperone, fluspirilene, penfluridol, pimozide, acepromazine, chlorpromazine, cyamemazine, dizyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, tiotixene, zuclopenthixol, clotiapine, loxapine, prothipendyl, carpipramine, clocapramine, molindone, mosapramine, sulpiride, veralipride, amisulpride, amoxapine, aripiprazole, asenapine, clozapine, blonanserin, iloperidone, lurasidone, melperone, nemonapride, olanzapine, paliperidone, perospirone, quetiapine, remoxipride, risperidone, sertindole, trimipramine, ziprasidone, zotepine, alstonie, befeprunox, bitopertin, brexpiprazole, cannabidiol, cariprazine, pimavanserin, pomaglumetad methionil, vabicaserin, xanomeline, and zicronapine.

Exemplary analgesics include, but are not limited to, paracetamol/acetaminophen, nonsteroidal anti-inflammants (e.g. ibuprofen, naproxen, ketoprofen, and nimesulide), COX-2 inhibitors (e.g. rofecoxib, celecoxib, and etoricoxib), opioids (e.g. morphine, codeine, oxycodone, hydrocodone, dihydromorphine, pethidine, buprenorphine), tramadol, norepinephrine, flupiretine, nefopam, orphenadrine, pregabalin, gabapentin, cyclobenzaprine, scopolamine, methadone, ketobemidone, piritramide, and aspirin and related salicylates (e.g. choline salicylate, magnesium salicylate, and sodium salicylate).

Exemplary antispasmodics include, but are not limited to, mebeverine, papverine, cyclobenzaprine, carisoprodol, orphenadrine, tizanidine, metaxalone, methodcarbamol, chlorzoxazone, baclofen, dantrolene, baclofen, tizanidine, and dantrolene. Suitable anti-inflammatories include, but are not limited to, prednisone, non-steroidal anti-inflammants (e.g. ibuprofen, naproxen, ketoprofen, and nimesulide), COX-2 inhibitors (e.g. rofecoxib, celecoxib, and etoricoxib), and immune selective anti-inflammatory derivatives (e.g. submandibular gland peptide-T and its derivatives).

Exemplary anti-histamines include, but are not limited to, H1-receptor antagonists (e.g. acrivastine, azelastine, bilastine, brompheniramine, buclizine, bromodiphenhydramine, carbinoxamine, cetirizine, chlorpromazine, cyclizine, chlorpheniramine, clemastine, cyproheptadine, desloratadine, dexbromapheniramine, dexchlorpheniramine, dimenhydrinate, dimetindene, diphenhydramine, doxylamine, ebasine, embramine, fexofenadine, hydroxyzine, levocetirzine, loratadine, meclozine, mirtazapine, olopatadine, orphenadrine, phenindamine, pheniramine, phenyltoloxamine, promethazine, pyrilamine, quetiapine, rupatadine, tripelennamine, and triprolidine), H2-receptor antagonists (e.g. cimetidine, famotidine, lafutidine, nizatidine, rafitidine, and roxatidine), tritoqualine, catechin, cromoglicate, nedocromil, and p2-adrenergic agonists.

Exemplary anti-infectives include, but are not limited to, amebicides (e.g. nitazoxanide, paromomycin, metronidazole, tinidazole, chloroquine, miltefosine, amphotericin b, and iodoquinol), aminoglycosides (e.g. paromomycin, tobramycin, gentamicin, amikacin, kanamycin, and neomycin), anthelmintics (e.g. pyrantel, mebendazole, ivermectin, praziquantel, abendazole, thiabendazole, oxamniquine), antifungals (e.g. azole antifungals (e.g. itraconazole, fluconazole, posaconazole, ketoconazole, clotrimazole, miconazole, and voriconazole), echinocandins (e.g. caspofungin, anidulafungin, and micafungin), griseofulvin, terbinafine, flucytosine, and polyenes (e.g. nystatin, and amphotericin b), antimalarial agents (e.g. pyrimethamine/sulfadoxine, artemether/lumefantrine, atovaquone/proquanil, quinine, hydroxychloroquine, mefloquine, chloroquine, doxycycline, pyrimethamine, and halofantrine), antituberculosis agents (e.g. aminosalicylates (e.g. aminosalicylic acid), isoniazid/rifampin, isoniazid/pyrazinamide/rifampin, bedaquiline, isoniazid, ethambutol, rifampin, rifabutin, rifapentine, capreomycin, and cycloserine), antivirals (e.g. amantadine, rimantadine, abacavir/lamivudine, emtricitabine/tenofovir, cobicistat/elvitegravir/emtricitabine/tenofovir, efavirenz/emtricitabine/tenofovir, avacavir/lamivudine/zidovudine, lamivudine/zidovudine, emtricitabine/tenofovir, emtricitabine/opinavir/ritonavir/tenofovir, interferon alfa-2v/ribavirin, peginterferon alfa-2b, maraviroc, raltegravir, dolutegravir, enfuvirtide, foscarnet, fomivirsen, oseltamivir, zanamivir, nevirapine, efavirenz, etravirine, rilpivirine, delaviridine, nevirapine, entecavir, lamivudine, adefovir, sofosbuvir, didanosine, tenofovir, avacivr, zidovudine, stavudine, emtricitabine, xalcitabine, telbivudine, simeprevir, boceprevir, telaprevir, lopinavir/ritonavir, fosamprenvir, dranuavir, ritonavir, tipranavir, atazanavir, nelfinavir, amprenavir, indinavir, sawuinavir, ribavirin, valacyclovir, acyclovir, famciclovir, ganciclovir, and valganciclovir), carbapenems (e.g. doripenem, meropenem, ertapenem, and cilastatin/imipenem), cephalosporins (e.g. cefadroxil, cephradine, cefazolin, cephalexin, cefepime, ceflaroline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, cefizoxime, and ceftazidime), glycopeptide antibiotics (e.g. vancomycin, dalbavancin, oritavancin, and telvancin), glycylcyclines (e.g. tigecycline), leprostatics (e.g. clofazimine and thalidomide), lincomycin and derivatives thereof (e.g. clindamycin and lincomycin), macrolides and derivatives thereof (e.g. telithromycin, fidaxomicin, erythromycin, azithromycin, clarithromycin, dirithromycin, and troleandomycin), linezolid, sulfamethoxazole/trimethoprim, rifaximin, chloramphenicol, fosfomycin, metronidazole, aztreonam, bacitracin, penicillins (amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxacillin, dicloxacillin, and nafcillin), quinolones (e.g. lomefloxacin, norfloxacin, ofloxacin, qatifloxacin, moxifloxacin, ciprofloxacin, levofloxacin, gemifloxacin, moxifloxacin, cinoxacin, nalidixic acid, enoxacin, grepafloxacin, gatifloxacin, trovafloxacin, and sparfloxacin), sulfonamides (e.g. sulfamethoxazole/trimethoprim, sulfasalazine, and sulfasoxazole), tetracyclines (e.g. doxycycline, demeclocycline, minocycline, doxycycline/salicylic acid, doxycycline/omega-3 polyunsaturated fatty acids, and tetracycline), and urinary anti-infectives (e.g. nitrofurantoin, methenamine, fosfomycin, cinoxacin, nalidixic acid, trimethoprim, and methylene blue).

Exemplary chemotherapeutics include, but are not limited to, paclitaxel, brentuximab vedotin, doxorubicin, 5-FU (fluorouracil), everolimus, pemetrexed, melphalan, pamidronate, anastrozole, exemestane, nelarabine, ofatumumab, bevacizumab, belinostat, tositumomab, carmustine, bleomycin, bosutinib, busulfan, alemtuzumab, irinotecan, vandetanib, bicalutamide, lomustine, daunorubicin, clofarabine, cabozantinib, dactinomycin, ramucirumab, cytarabine, Cytoxan, cyclophosphamide, decitabine, dexamethasone, docetaxel, hydroxyurea, decarbazine, leuprolide, epirubicin, oxaliplatin, asparaginase, estramustine, cetuximab, vismodegib, asparginase Erwinia chrysanthemi, amifostine, etoposide, flutamide, toremifene, fulvestrant, letrozole, degarelix, pralatrexate, methotrexate, floxuridine, obinutuzumab, gemcitabine, afatinib, imatinib mesylatem, carmustine, eribulin, trastuzumab, altretamine, topotecan, ponatinib, idarubicin, ifosfamide, ibrutinib, axitinib, interferon alfa-2a, gefitinib, romidepsin, ixabepilone, ruxolitinib, cabazitaxel, ado-trastuzumab emtansine, carfilzomib, chlorambucil, sargramostim, cladribine, mitotane, vincristine, procarbazine, megestrol, trametinib, mesna, strontium-89 chloride, mechlorethamine, mitomycin, busulfan, gemtuzumab ozogamicin, vinorelbine, filgrastim, pegfilgrastim, sorafenib, nilutamide, pentostatin, tamoxifen, mitoxantrone, pegaspargase, denileukin diftitox, alitretinoin, carboplatin, pertuzumab, cisplatin, pomalidomide, prednisone, aldesleukin, mercaptopurine, zoledronic acid, lenalidomide, rituximab, octretide, dasatinib, regorafenib, histrelin, sunitinib, siltuximab, omacetaxine, thioguanine (tioguanine), dabrafenib, erlotinib, bexarotene, temozolomide, thiotepa, thalidomide, BCG, temsirolimus, bendamustine hydrochloride, triptorelin, aresnic trioxide, lapatinib, valrubicin, panitumumab, vinblastine, bortezomib, tretinoin, azacitidine, pazopanib, teniposide, leucovorin, crizotinib, capecitabine, enzalutamide, ipilimumab, goserelin, vorinostat, idelalisib, ceritinib, abiraterone, epothilone, tafluposide, azathioprine, doxifluridine, vindesine, and all-trans retinoic acid.

Exemplary radiation sensitizers include, but are not limited to, 5-fluorouracil, platinum analogs (e.g. cisplatin, carboplatin, and oxaliplatin), gemcitabine, DNA topoisomerase I-targeting drugs (e.g. camptothecin derivatives (e.g. topotecan and irinotecan)), epidermal growth factor receptor blockade family agents (e.g. cetuximab, gefitinib), farnesyltransferase inhibitors (e.g., L-778-123), COX-2 inhibitors (e.g. rofecoxib, celecoxib, and etoricoxib), bFGF and VEGF targeting agents (e.g. bevazucimab and thalidomide), NBTXR3, Nimoral, trans sodium crocetinate, NVX-108, and combinations thereof. See also e.g., Kvols, L. K., J Nucl Med 2005; 46:187S-190S.

### Optimization of Test Compound Pools

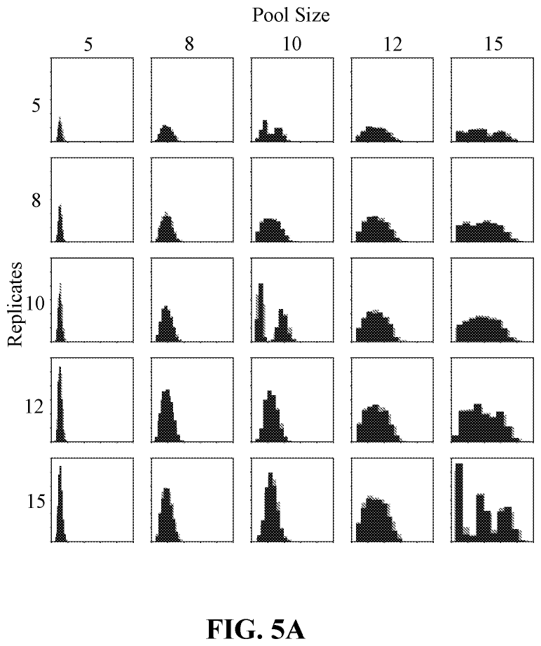

The pools of test compounds can be optimized. In some embodiments, a group of test compounds to be selected for composing subsets can be pre-screened or pre-selected to have maximal structure distinctiveness. This approach can substantially reduce the chemical similarity among the test compounds. In a preferred embodiment, a library of chemically distinct test compounds can be used for selecting for subsets. As a result, the chemical similarity will not change substantially after the optimization process as depicted in FIG. 5. In some embodiments, the KS distance between mean connectivities per pool before and after optimization is composed. In some embodiments, the KS distances composed in such way show the effect of chemical similarity on biological connectivity is minimal after the optimization process disclosed herein.

In some embodiments, test compounds without pre-screening for maximizing structure distinctiveness are used for selecting for forming subsets. The chemical similarity among the test compounds can be reduced by not placing test compounds with high structure similarity into the same pool. In some embodiments, an increase in replicates, i.e. an increase in the number of a given test compound appearing in different pool, will not increase the chemical similarity in a given pool. In some embodiments, the chemical similarity in a given pool is not impacted substantially by an increase in the pool size, i.e. the number of test targets in a given pool.

In some embodiments, the biological connectivity of test compounds in a given pool is reduced substantially with the optimization methods disclosed herein. In some embodiments, as the number of test compounds in a given pool (the pool size) increases, the biological connectivity of the given pool will increase. In some embodiments, as the number of replicates for a given test compounds increases, the biological connectivity of a given pool will not increase. This characteristic provides great benefit for the use of many replicates for a given test compound, while maintaining the pool size to a certain number. In some embodiments, the Kolmogorov-Smirnov (KS) distance between mean connectivity can be used to show the changes before and after optimization. The KS distance is a statistical term that refers to the distance between the empirical distribution function of a sample and the cumulative distribution function of a reference distribution, or between the empirical distribution functions of two samples.

In some embodiments, the optimization method disclosed herein produces rear repeated drug pairing. In some embodiments, the repeated drug pairing does not change while the number of replicates increases for a given test compound. In some embodiments, the repeated drug pairing increases as the pool size increases.

### Methods of Screening Using Pools of Target Compounds

The pools of target compounds described herein can be used in various screening methods, including high-throughput methods, where identifying the effects of a pool of compounds or one or more specific compounds within a pool on a subject is useful and/or advantageous. Without limitation, the pools of target compounds described elsewhere herein can allow for screening methods that are more efficient and cheaper that previous compound screening methods. Such methods can be used to identify, for example, one or more effects of one or more of the test compounds on the subject, therapies for disease, methods and compounds for directing cell differentiation, identifying compound(s) capable of causing a beneficial functional change (e.g. improve growth rate, improve muscle mass, improve fat content, increase hair growth, etc.) that may or may not be associated with a disease state, among others. In some embodiments, the method of screening described herein can be used in a personalized medicine context, such as when the sample used in the screen is obtained from the specific human or non-human animal or plant in need of treatment. Generally, the methods include contacting a biological sample with a pool of test compounds and subsequently detecting a change in the sample in response to the pool of test compounds. In some embodiments, the method includes determining the test compound(s) generating the observed effect. In some embodiments, the methods can further include identifying one or more therapy targets, therapies, methods of cell differentiation, and/or diagnosing, prognosing, and or monitoring a disease based on the observed effect (or lack thereof), effective test compounds identified, or combination thereof.

### Subjects

The screening methods herein can generally include contacting a subject with a pool of test compounds as previously described, such as an optimized pool of test compounds. In this context, the term “test subject” or “subject” refers to a biological sample such as a cell, cell population, tissue, organoid, or other biological sample. In some embodiments, the biological sample, such as the cell, cell population, tissue, organoid, or other biological sample can be obtained, derived, isolated, or otherwise generated from a cell, cell population, tissue, organ, of a human or non-human animal. In some embodiments, a cell used as test subject for screening can be a stem cell, a cancer cell, a primary cultured cell, a cell line, a cell derived from an animal or a human, a cell with genetic modifications, a cell cultured in attachment manner, or a cell cultured in suspension manner. In some embodiments, the cell is a stem cell. In some embodiments, the stem cell is derived from tissues comprising the intestine, liver, spleen, pancreas, stomach, esophageal, skin, eye, central nervous system, peripheral nervous system, kidney, ovary, breast, testis, uterus, bone, cartilage, bone marrow, peripheral blood, lymph node, thymus, and the lung. In some embodiments, the tissue used as test subject for screening can be an organoid, a biopsy from an animal or a human, or a dissected tissue from an animal or a human. The tissue used herein comprises the intestine, liver, spleen, pancreas, stomach, esophageal, skin, eye, central nervous system, peripheral nervous system, kidney, ovary, breast, testis, uterus, bone, cartilage, bone marrow, peripheral blood, lymph node, thymus, and the lung.

In some embodiments, the test subject for screening is an organoid. The organoid can be derived from stem cells from a variety of tissues or organs. The source of stem cells comprises the intestine, liver, spleen, pancreas, stomach, esophageal, skin, eye, central nervous system, peripheral nervous system, kidney, ovary, breast, testis, uterus, bone, cartilage, bone marrow, peripheral blood, lymph node, thymus, and the lung. The term “organoid” refers to an in vitro collection of cells that resemble their in vivo counterparts and form 3D structures. In some embodiments, the organoids of the assay are mammalian organoids, for example human or murine organoids i.e. they are derived from cells taken from a mammal. The mammal may be any mammal of interest, for example a human or mouse. In some embodiments the organoids are non-human. In a preferred embodiment, the organoids are derived from human. In some embodiments, the organoids of the assay are epithelial organoids or endothelial organoids. In a preferred embodiment the organoids are epithelial organoids. In some embodiments, the organoids do not comprise non-epithelial cells, i.e. the only cell type present in the organoid is an epithelial cell. The organoids of the assay typically comprise a lumen, preferably a closed lumen. The cells of the organoid typically form an epithelial layer or endothelial layer around the lumen and the cells of the epithelial layer or endothelial layer are polarized.

In some embodiments, the organoids used for the screening are derived from gastric, intestinal (for example, small intestinal, colonic, rectum, duodenum or ileum), pancreatic, prostate, lung, breast, kidney, blood vessel or lymphatic vessel organoids. This typically means that the organoids are derived from gastric, intestinal (for example, small intestinal, colonic, rectum, duodenum or ileum), pancreatic, prostate, liver, pancreas, brain, ovary, uterus, skin, esophagus, lung, spleen, kidney, bone, bone marrow, brain, central nervous system, peripheral nervous system, blood, cartilage, fat, and eye lung, breast, kidney, blood vessel or lymphatic vessel cells respectively. In a preferred embodiment, the organoids used for the screening of a plurality of test compounds herein are derived stems cells from the intestine of mammals comprising humans and mice.

In some embodiments, a skilled person in the art will understand that there may be alternative ways of generating an organoid that has an in vivo genotype and phenotype. Thus, an organoid that has the in vivo genotype and phenotype of the intestine is for the purposes of this invention comprised within the definition of an intestinal organoid. The same applies for the other organoid types listed above. In some embodiments, the one or more organoids are intestinal or lung organoids. The term “resembles” means that the organoid has genetic and phenotypic characteristics that allow it to be recognized by a skilled person in the art as being from or associated with a particular tissue type (such as the tissues listed above). It does not mean that the organoid necessarily has to be genetically and phenotypically identical (or thereabouts) to the corresponding in vivo tissue cell type. However, in a preferred embodiment, the organoids used in the assay comprise cells that are genetically and phenotypically stable relative to the in vivo cell or cells that the organoid was derived from. By genetically and phenotypically stable, it is meant that there is no genetic manipulation involved.

### Effect Analysis

The methods of screening herein generally involve detecting, measuring, or otherwise analyzing one or more effects of one or more compounds of the pool of test compounds that the subject was exposed to. In some embodiments, the effect measured is a change in genotype or phenotype of the subject at the bulk or single cell level. A change in genotype can be measured by a variety of suitable analytic techniques such as a suitable bulk and/or single cell sequencing technique. A change in phenotype can be measured by using one or more of a variety of techniques capable of analyzing the epigenome, transcriptome, proteome, metabolome, secretome, combinations thereof and the like. The effect measured can also be a change in cell type or cell state. In some embodiments, one or more biomarkers, signatures, and the like can be used to identify a phenotype, cell type, and/or cell state. Biomarkers in the context of the present invention encompasses, without limitation nucleic acids, proteins, reaction products, and metabolites, together with their polymorphisms, mutations, variants, modifications, subunits, fragments, and other analytes or sample-derived measures. In certain embodiments, biomarkers include the signature genes or signature gene products, and/or cells as described herein.

Biomarkers are useful in methods of diagnosing, prognosing and/or staging an immune response in a subject by detecting a first level of expression, activity and/or function of one or more biomarker and comparing the detected level to a control of level wherein a difference in the detected level and the control level indicates that the presence of an immune response in the subject.

As used herein, “cell type” refers to the more permanent aspects (e.g. a hepatocyte typically can't on its own turn into a neuron) of a cell's identity. Cell state can be thought of as the permanent characteristic profile or phenotype of a cell. Cell types are often organized in a hierarchical taxonomy, types may be further divided into finer subtypes; such taxonomies are often related to a cell fate map, which reflect key steps in differentiation or other points along a development process. Wagner et al., 2016. Nat Biotechnol. 34(111): 1145-1160.

As used herein, “cell state” are used to describe transient elements of a cell's identity. Cell state can be thought of as the transient characteristic profile or phenotype of a cell. Cell states arise transiently during time-dependent processes, either in a temporal progression that is unidirectional (e.g., during differentiation, or following an environmental stimulus) or in a state vacillation that is not necessarily unidirectional and in which the cell may return to the origin state. Vacillating processes can be oscillatory (e.g., cell-cycle or circadian rhythm) or can transition between states with no predefined order (e.g., due to stochastic, or environmentally controlled, molecular events). These time-dependent processes may occur transiently within a stable cell type (as in a transient environmental response), or may lead to a new, distinct type (as in differentiation). Wagner et al., 2016. Nat Biotechnol. 34(111): 1145-1160.

As used herein, “phenotype” refers to the configuration of observable or measurable trait(s) of the subject being evaluated or measure (e.g. a cell, cell population, tissue, organoid and the like).

As used herein, “evaluate” refers to assessing, such as by any suitable objective method of measuring, a characteristic, output, response, or other value or trait of a subject to be assessed.

As used herein, “observable trait” refers to any characteristic of a cell, population of cell, tissue, organ, organ system, and/or organism that is measurable or otherwise observable. An observable trait can be, for example, gene expression, protein expression, epigenetic status or signature, functionality, morphology, temporal and/or spatial localization. An observable trait or traits can define a phenotype.

As used herein a “signature” may encompass any gene or genes, protein or proteins, or epigenetic element(s) whose expression profile or whose occurrence is associated with a specific cell type, subtype, or cell state of a specific cell type or subtype within a population of cells. For ease of discussion, when discussing gene expression, any of gene or genes, protein or proteins, or epigenetic element(s) may be substituted. As used herein, the terms “signature”, “expression profile”, or “expression program” may be used interchangeably. It is to be understood that also when referring to proteins (e.g. differentially expressed proteins), such may fall within the definition of “gene” signature. Levels of expression or activity or prevalence may be compared between different cells in order to characterize or identify for instance signatures specific for cell (sub)populations. Increased or decreased expression or activity or prevalence of signature genes may be compared between different cells in order to characterize or identify for instance specific cell (sub)populations. The detection of a signature in single cells may be used to identify and quantitate for instance specific cell (sub)populations. A signature may include a gene or genes, protein or proteins, or epigenetic element(s) whose expression or occurrence is specific to a cell (sub)population, such that expression or occurrence is exclusive to the cell (sub)population. A gene signature as used herein, may thus refer to any set of up- and down-regulated genes that are representative of a cell type or subtype. A gene signature as used herein, may also refer to any set of up- and down-regulated genes between different cells or cell (sub)populations derived from a gene-expression profile. For example, a gene signature may comprise a list of genes differentially expressed in a distinction of interest.

The signature as defined herein (being it a gene signature, protein signature or other genetic or epigenetic signature) can be used to indicate the presence of a cell type, a subtype of the cell type, the state of the microenvironment of a population of cells, a particular cell type population or subpopulation, and/or the overall status of the entire cell (sub)population. Furthermore, the signature may be indicative of cells within a population of cells in vivo. The signature may also be used to suggest for instance particular therapies, or to follow up treatment, or to suggest ways to modulate immune systems. The signatures of the present invention may be discovered by analysis of expression profiles of single-cells within a population of cells from isolated samples (e.g. blood samples), thus allowing the discovery of novel cell subtypes or cell states that were previously invisible or unrecognized. The presence of subtypes or cell states may be determined by subtype specific or cell state specific signatures. The presence of these specific cell (sub)types or cell states may be determined by applying the signature genes to bulk sequencing data in a sample. Not being bound by a theory the signatures of the present invention may be microenvironment specific, such as their expression in a particular spatio-temporal context. Not being bound by a theory, signatures as discussed herein are specific to a particular pathological context. Not being bound by a theory, a combination of cell subtypes having a particular signature may indicate an outcome. Not being bound by a theory, the signatures can be used to deconvolute the network of cells present in a particular pathological condition. Not being bound by a theory, the presence of specific cells and cell subtypes are indicative of a particular response to treatment, such as including increased or decreased susceptibility to treatment. The signature may indicate the presence of one particular cell type. In one embodiment, the novel signatures are used to detect multiple cell states or hierarchies that occur in subpopulations of cancer cells that are linked to particular pathological condition (e.g. cancer grade), or linked to a particular outcome or progression of the disease, or linked to a particular response to treatment of the disease.

The signature according to certain embodiments of the present invention may comprise or consist of one or more genes, proteins and/or epigenetic elements, such as for instance 1, 2, 3, 4, 5, 6, 7, 8, 9, 10 or more. In certain embodiments, the signature may comprise or consist of two or more genes, proteins and/or epigenetic elements, such as for instance 2, 3, 4, 5, 6, 7, 8, 9, 10 or more. In certain embodiments, the signature may comprise or consist of three or more genes, proteins and/or epigenetic elements, such as for instance 3, 4, 5, 6, 7, 8, 9, 10 or more. In certain embodiments, the signature may comprise or consist of four or more genes, proteins and/or epigenetic elements, such as for instance 4, 5, 6, 7, 8, 9, 10 or more. In certain embodiments, the signature may comprise or consist of five or more genes, proteins and/or epigenetic elements, such as for instance 5, 6, 7, 8, 9, 10 or more. In certain embodiments, the signature may comprise or consist of six or more genes, proteins and/or epigenetic elements, such as for instance 6, 7, 8, 9, 10 or more. In certain embodiments, the signature may comprise or consist of seven or more genes, proteins and/or epigenetic elements, such as for instance 7, 8, 9, 10 or more. In certain embodiments, the signature may comprise or consist of eight or more genes, proteins and/or epigenetic elements, such as for instance 8, 9, 10 or more. In certain embodiments, the signature may comprise or consist of nine or more genes, proteins and/or epigenetic elements, such as for instance 9, 10 or more. In certain embodiments, the signature may comprise or consist of ten or more genes, proteins and/or epigenetic elements, such as for instance 10, 11, 12, 13, 14, 15, or more. It is to be understood that a signature according to the invention may for instance also include genes or proteins as well as epigenetic elements combined.

In certain embodiments, a signature is characterized as being specific for a particular tumor cell or tumor cell (sub)population if it is upregulated or only present, detected or detectable in that particular tumor cell or tumor cell (sub)population, or alternatively is downregulated or only absent, or undetectable in that particular tumor cell or tumor cell (sub)population. In this context, a signature consists of one or more differentially expressed genes/proteins or differential epigenetic elements when comparing different cells or cell (sub)populations, including comparing different tumor cells or tumor cell (sub)populations, as well as comparing tumor cells or tumor cell (sub)populations with non-tumor cells or non-tumor cell (sub)populations. It is to be understood that “differentially expressed” genes/proteins include genes/proteins which are up- or down-regulated as well as genes/proteins which are turned on or off. When referring to up-or down-regulation, in certain embodiments, such up- or down-regulation is preferably at least two-fold, such as two-fold, three-fold, four-fold, five-fold, or more, such as for instance at least ten-fold, at least 20-fold, at least 30-fold, at least 40-fold, at least 50-fold, or more. Alternatively, or in addition, differential expression may be determined based on common statistical tests, as is known in the art.

As discussed herein, differentially expressed genes/proteins, or differential epigenetic elements may be differentially expressed on a single cell level, or may be differentially expressed on a cell population level. Preferably, the differentially expressed genes/proteins or epigenetic elements as discussed herein, such as constituting the gene signatures as discussed herein, when as to the cell population level, refer to genes that are differentially expressed in all or substantially all cells of the population (such as at least 80%, preferably at least 90%, such as at least 95% of the individual cells). This allows one to define a particular subpopulation of tumor cells. As referred to herein, a “subpopulation” of cells preferably refers to a particular subset of cells of a particular cell type which can be distinguished or are uniquely identifiable and set apart from other cells of this cell type. The cell subpopulation may be phenotypically characterized, and is preferably characterized by the signature as discussed herein. A cell (sub)population as referred to herein may constitute of a (sub)population of cells of a particular cell type characterized by a specific cell state.

When referring to induction, or alternatively suppression of a particular signature, preferable is meant induction or alternatively suppression (or upregulation or downregulation) of at least one gene/protein and/or epigenetic element of the signature, such as for instance at least to, at least three, at least four, at least five, at least six, or all genes/proteins and/or epigenetic elements of the signature.

Signatures may be functionally validated as being uniquely associated with a particular immune responder phenotype. Induction or suppression of a particular signature may consequentially be associated with or causally drive a particular immune responder phenotype.

Various aspects and embodiments of the invention may involve analyzing gene signatures, protein signature, and/or other genetic or epigenetic signature based on single cell analyses (e.g. single cell RNA sequencing) or alternatively based on cell population analyses, as is defined herein elsewhere.

In further aspects, the invention relates to gene signatures, protein signature, and/or other genetic or epigenetic signature of particular tumor cell subpopulations, as defined herein elsewhere. The invention hereto also further relates to particular tumor cell subpopulations, which may be identified based on the methods according to the invention as discussed herein; as well as methods to obtain such cell (sub)populations and screening methods to identify agents capable of inducing or suppressing particular tumor cell (sub)populations.

The term “screening” as used herein, refers to the analysis or investigation of one or more agents as part of a methodical survey to assess suitability of the one or more agents for a particular purpose or to identify a previously unknown function, activity, or effect. In this context, the term “screening” has the same meaning as “evaluating”, “evaluation”, “identifying”, and “identification”. Such terms are used in this context interchangeably.

### Exemplary Effect Analysis Techniques

As previously discussed, any suitable detection and/or analytic technique can be used to detect one or more effects of the pool of test compound on the subject. Suitable techniques include, but are not limited to, those capable of measuring and/or detecting (quantitatively or qualitatively) an effect on the genome, transcriptome, proteome, epigenome, secretome, metabolome, combinations thereof, and the like. Exemplary techniques are described herein and will also be appreciated by those of skill in the art.

### Polynucleotide Sequencing Methods

In some embodiments, the methods herein may comprise sequencing isolated nucleic acids, including DNA and RNA. Suitable sequencing techniques include, but are not limited to, using automated Sanger sequencing (AB13730xl genome analyzer), pyrosequencing on a solid support (454 sequencing, Roche), sequencing-by-synthesis with reversible terminations (ILLUMINA® Genome Analyzer), sequencing-by-ligation (ABI SOLiD®) or sequencing-by-synthesis with virtual terminators (HELISCOPE®); Moleculo sequencing (see Voskoboynik et al. eLife 2013 2:e00569 and U.S. patent application Ser. No. 13/608,778, filed Sep. 10, 2012); DNA nanoball sequencing; Single molecule real time (SMRT) sequencing; Nanopore DNA sequencing; Sequencing by hybridization; Sequencing with mass spectrometry; and Microfluidic Sanger sequencing. Examples of information that can be obtained from the disclosed methods and the analysis of the results thereof, include without limitation uni- or multiplex, three-dimensional genome mapping, genome assembly, one dimensional genome mapping, the use of single nucleotide polymorphisms to phase genome maps, for example to determine the patterns of chromosome inactivation, such as for analysis of genomic imprinting, the use of specific junctions to determine karyotypes, including, but not limited to, chromosome number alterations (such as unisomies, uniparental disomies, and trisomies), translocations, inversions, duplications, deletions and other chromosomal rearrangements, the use of specific junctions correlated with disease to aid in diagnosis. As would be apparent, forward and reverse sequencing primer sites that are compatible with a selected next generation sequencing platform can be added to the ends of the fragments during the amplification step. In certain embodiments, the fragments may be amplified using PCR primers that hybridize to the tags that have been added to the fragments, where the primer used for PCR have 5′ tails that are compatible with a particular sequencing platform. In certain cases, the primers used may contain a molecular barcode (an “index”) so that different pools can be pooled together before sequencing, and the sequence reads can be traced to a particular sample using the barcode sequence.

In some cases, the sequencing may be next generation sequencing. The terms “next-generation sequencing” or “high-throughput sequencing” refer to the so-called parallelized sequencing-by-synthesis or sequencing-by-ligation platforms currently employed by Illumina, Life Technologies, and Roche, etc. Next-generation sequencing methods may also include nanopore sequencing methods or electronic-detection based methods such as Ion Torrent technology commercialized by Life Technologies or single-molecule fluorescence-based method commercialized by Pacific Biosciences. Any method of sequencing known in the art can be used before and after isolation. In certain embodiments, a sequencing library is generated and sequenced. In some examples, the sequencing is performed by transporting the fragments through an orifice in an electric field and measuring change of an electric current density across the orifice when the fragments are transported. The diameter of the orifice may be from 0.1 nm to 10 μm, e.g., from 0.1 nm to 1 nm, 0.5 nm to 5 nm, 1 nm to 10 nm, 10 nm to 100 nm, 100 nm to 1 μm, 1 to 10 μm. Such sequencing method may be a nanopore DNA sequencing method. Examples of nanopore DNA sequencing methods are described in nanoporetech.com/applications/epigenetics.

In some cases, the sequencing may be performed at certain “depth.” The terms “depth” or “coverage” as used herein refers to the number of times a nucleotide is read during the sequencing process. In regards to single cell RNA sequencing, “depth” or “coverage” as used herein refers to the number of mapped reads per cell. Depth, in regards to genome sequencing, may be calculated from the length of the original genome (G), the number of reads (N), and the average read length (L) as N×L/G. For example, a hypothetical genome with 2,000 base pairs reconstructed from 8 reads with an average length of 500 nucleotides will have 2× redundancy.

In some cases, the sequencing herein may be low-pass sequencing. The terms “low-pass sequencing” or “shallow sequencing” as used herein refers to a wide range of depths greater than or equal to 0.1×up to 1×. Shallow sequencing may also refer to about 5000 reads per cell (e.g., 1,000 to 10,000 reads per cell).

In some cases, the sequencing herein may deep sequencing or ultra-deep sequencing. The term “deep sequencing” as used herein indicates that the total number of reads is many times larger than the length of the sequence under study. The term “deep” as used herein refers to a wide range of depths greater than 1× up to 100×. Deep sequencing may also refer to 100× coverage as compared to shallow sequencing (e.g., 100,000 to 1,000,000 reads per cell). The term “ultra-deep” as used herein refers to higher coverage (>100-fold), which allows for detection of sequence variants in mixed populations.

In some embodiments, a droplet-based sequencing technique can be used. Exemplary droplet-based sequencing techniques include Drop-Seq (see e.g. International Patent Publication No. WO2016/040476), Single cell Droplet-based RNA sequencing (see e.g., Salomon et al., 2019. Lab on a Chip. 19:1706-1727; Klein et al., Cell. 2015 May 21; 161(5): 1187-1201; Klein and Macosko. DOI: 10.1039/C7LC90070H (Editorial) Lab Chip, 2017, 17, 2540-2541; Zhang et al., 2019. Molecular Cell. 73:130-142; Lan et al., Nature Communications volume 7, Article number: 11784 (2016), which can be adapted for use with the embodiments disclosed herein).

In some embodiments, a bead-based sequencing technique can be used. In some embodiments, the beads can be barcoded using a suitable barcoding technique. See e.g. Zilionis et al., Nat. Prot. 2017. 12(1): 44-73. doi:10.1038/nprot.2016.154; Tambe and Pachter. 2019. BCM Bioinfor. 20,32. https://doi.org/10.1186/s12859-019-2612-0; Cheng et al., 2019. 10.1038/protex.2018.116, which can be adapted for use with the embodiments described herein.

In some embodiments, a single cell sequencing technique can be used. Exemplary single cell sequencing techniques include, but are not limited to, those set forth in WO2016/040476, Chen et al., Front. Genet., 5 Apr. 2019 https://doi.org/10.3389/fgene.2019.00317, particularly at Table 1, scRNA-seq, SUPeR-seq, MATQ-seq, RamDA-seq, SINC-seq, ViscRNA-seq, UMI Methods, Digital RNA HiRes-SEQ, FREQ-SEQ RNAtag-Seq, MARS-Seq, Quartz-Seq, Quartz-Seq2, DP-Seq, Smart-Seq, Nano-Cage, Smart-Seq2, snRNA-Seq, FRISCR, SPLiT-seq, sci-RNA-seq, CEL-seq, STRT, TCR Chain pairing, TCR-LA-MC PCR, CirSeq, TIVA, PAIR, CLaP, CytoSeq, Drop-Seq, snDrop-Seq, DroNC-Seq, CITE-Seq, ECCITE-Seq, CROP-Seq, Mosaic-Seq, Act-Seq, Seq-Well, Microwell-seq, Nanogrid-SNRS, Multi-Seq, Hi-SCL, in-Drop, Nuc-Seq, Div-Seq, SCRB-Seq, smMIP, MIPSTR, MDA, IMS-MDA, MIDAS, SCMDA, MALBAC, SNES, LIANTI, Sci-DNA-Seq, CRISPR-UMI, TSCS, OS-Seq, Safe-SeqS, Duplex-Seq, snmC-Seq, scAba-Seq, sci-MET, scRC-Seq, scChIP-seq, scATAC-Seq, Drop-ChIP, scTHS-seq, sciHi-C, Dip-C, SMDB, SIDR, DR-Seq, G&T-Seq, scM&T-Seq, sci-CAR, scTrio-Seq, scTrio-Seq2, scNMT-seq, scCool-Seq, TruSeq PCR Free, TruSeqNano, AmpliSeq, TruSeq RNA, TruSeq small RNA, TruSeq stranded RNA, TruSeq RNA exome, TruSeq targeted RNA expression, and combinations thereof, which can be adapted for use with the embodiments described herein.

In some embodiments, an electrochemical polynucleotide sequencing method can be used such as that described in U.S. Patent Publication No. US20190137435, which can be adapted for use with the embodiments disclosed herein.

In some embodiments, an electric field assisted sequencing method can be used. Exemplary electric field assisted sequencing method include, but are not limited to, those described in e.g. Sigalov et al. Nano Lett. 2008, 8, 1, 56-63; Applied Physics Letters 82(8):1308-1310, March 2003, which can be adapted for use with the embodiments disclosed herein.

In some embodiments a single-cell sequencing technique is used.

In some embodiments, sequencing comprises a single cell or single nucleus sequencing technique or component thereof, or both. Exemplary single cell and single nucleus sequencing techniques include, but are not limited to, Act-Seq (see e.g. Wu Y. E. et al. (2017) Neuron 96(2): 313-329); CEL-Seq (see e.g., Hashimshony T. et al. (2012) Cell Rep 2: 666-673); CirSeq (see e.g., Acevedo A. et al. (2014) Nature 505: 686-690); CITE-Seq (see e.g., Stoeckius M., et al. (2017) Nat Methods 14(9): 865-868); CLaP (see e.g., Binan L. et al. (2016) Nat Commun 7: 11636); CRISPR-UMI (see e.g., Michlits G. et al. (2017) Nat Methods 14(12): 1191-1197); CROP-Seq (see e.g., Datlinger P. et al. (2017) Nat Methods 14(3): 297-301); CytoSeq (see e.g., Fan H. C. et al. (2015) Science 347: 1258367); Digital RNA (see e.g., Shiroguchi K. et al. (2012) Proc Natl Acad Sci USA 109:1347-1352); Dip-C (see e.g., Tan L., et al. (2018) Science 361(6405): 924-928); Div-Seq (see e.g., Habib N. et al. (2016) Science 353(6302): 925-928); DP-Seq (see e.g., Bhargava V. et al. (2013) Sci Rep 3: 1740); DroNC-seq (see e.g., Habib N. et al. (2017) Nat Methods 14(10): 955-958); Drop-Seq (see e.g., Macosko E. Z. et al. (2015) Cell 161: 1202-1214); DR-Seq (see e.g., Dey S. S. et al. (2015) Nat Biotechnol 33: 285-9); Drop-ChIP (see e.g., Rotem A. et al. (2015) Nat Biotechnol 33: 1165-72); Duplex-Seq (see e.g., Schmitt M. W. et al. (2012) Proc Natl Acad Sci USA 109: 14508-14513); ECCITE-seq (see e.g., Mimitou E. P. et al. (2019) Nat Methods 16(5): 409-412); FREQ-Seq (see e.g., Chubiz L. M. et al. (2012) PLoS One 7: e47959); FRISCR (see e.g., Thomsen E. R. et al. (2016) Nat Methods 13: 87-93); G&T-seq (see e.g., Macaulay I. C. et al. (2015) Nat Methods 12: 519-522); HiRes-Seq (see e.g., Imashimizu M. et al. (2013) Nucleic Acids Res 41:9090-9104); Hi-SCL (see e.g., Rotem A. et al. (2015) PLoS One 10: e0116328); IMS-MDA (see e.g., Seth-Smith H. M. et al. (2013) Nat Protoc 8: 2404-2412); inDrop (see e.g., Klein A. M. et al. (2015) Cell 161: 1187-201); LIANTI (see e.g., Chen C. et al. (2017) Science 356(6334): 189-194); MALBAC (see e.g., Zong C. et al. (2012) Science 338: 1622-1626); MARS-seq (see e.g., Jaitin D. A. et al. (2014) Science 343:776-9); MATQ-seq (see e.g., Sheng K. et al. (2017) Nat Methods 14(3): 267-270); MDA (see e.g., Dean F. B. et al. (2001) Genome Res 11: 1095-1099); Microwell-seq (see e.g., Han X. et al. (2018) Cell 172(5): 1091-1107.e1017); MIDAS (see e.g., Gole J. et al. (2013) Nat Biotechnol 31:1126-32); MIPSTR (see e.g., Carlson K. D. et al. (2015) Genome Res 25: 750-761); Mosaic-seq (see e.g., Han X. et al. (2018) Cell 172(5): 1091-1107 e1017); MULTI-seq (see e.g., McGinnis C. S. et al. (2019) Nat Methods 16(7): 619-626); NanoCAGE (see e.g., Plessy C. et al. (2010) Nat Methods 7: 528-534); Nanogrid SNRS (see e.g., Gao R. et al. (2017) Nat Commun 8(1): 228); nuc-seq (see e.g., Wang Y. et al. (2014) Nature 512: 155-160); Nuc-Seq/SNES (see e.g., Leung M. L. et al. (2015) Genome Biology 16(1): 55); OS-Seq (see e.g., Myllykangas S. et al. (2011) Nat Biotechnol 29: 1024-1027); PAIR (see e.g., Bell T. J. et al. (2015) Methods Mol Biol 1324: 457-68); Quartz-Seq (see e.g., Sasagawa Y. et al. (2013) Genome Biol 14: R31); Quartz-Seq2 (see e.g., Sasagawa Y. et al. (2018) Genome Biology 19(1): 29); RamDA-seq (see e.g., Hayashi T. et al. (2018) Nature Communications 9(1): 619); RNAtag-Seq (see e.g., Shishkin A. A. et al. (2015) Nat Methods 12: 323-325); Safe-SeqS (see e.g., Kinde I. et al. (2011) Proc Natl Acad Sci USA 108: 9530-5); scABA-seq (see e.g., Mooijman D. et al. (2016) Nature Biotechnology 34: 852); scATAC-seq (see e.g., Buenrostro J. D. et al. (2015) Nature 523: 486-490 (Microfluidics)); scATAC-Seq (see e.g., Cusanovich D. A. et al. (2015) Science 348: 910-4 (Cell Index)); scChip-seq (see e.g., Rotem A. et al. (2015) Nat Biotechnol 33: 1165-72); scCool-seq (see e.g., Li L. et al. (2018) Nature Cell Biology 20(7): 847-858); sciHi-C (see e.g., Ramani V. et al. (2017) Nature Methods 14: 263); sci-CAR (see e.g., Cao J. et al. (2018) Science 361(6409): 1380); sci-DNA-seq (see e.g., Rosenberg A. B. et al. (2018) Science 360: 176-182); sci-MET (see e.g., Mulqueen R. M. et al. (2018) Nature Biotechnology 36: 428); sci-RNA-seq (see e.g., Cao J. et al. (2017) Science 357(6352): 661); SCMDA (see e.g., Dong X. et al. (2017) Nature Methods 14: 491); scM&T-seq (see e.g., Angermueller C. et al. (2016) Nature Methods 13: 229); scNIVIT-seq (see e.g., Clark S. J. et al. (2018) Nature Communications 9(1): 781 scRC-Seq Upton K. R. et al. (2015) Cell 161: 228-39); scRNA-seq (see e.g., Tang F. et al. (2009) Nat Methods 6: 377-82); SCRB-Seq Soumillon M. et al. (2014) bioRxiv: 003236); scTHS-seq (see e.g., Lake B. B. et al. (2018) Nature Biotechnology 36(1): 70-80); scTrio-seq (see e.g., Hou Y. et al. (2016) Cell Res 26: 304-19); scTrio-seq2 (see e.g., Bian S. et al. (2018) Science 362(6418): 1060); Seq-Well (see e.g., Gierahn T. M., et al. (2017). Nat Methods 14(4): 395-398); SIDR (see e.g., Han K. Y. et al. (2018) Genome Research 28(1): 75-87); SINC-seq (see e.g., Abdelmoez M. N. et al. (2018) Genome Biology 19(1): 66); Smart-Seq (see e.g., Ramskold D. et al. (2012) Nat Biotechnol 30: 777-782); Smart-seq2 (see e.g., Picelli S. et al. (2013) Nat Methods 10: 1096-1098v); SMDB (see e.g., Lan F. et al. (2016) Nat Commun 7: 11784); smMIP (see e.g., Hiatt J. B. et al. (2013) Genome Res 23: 843-854); snDrop-seq (see e.g., Lake B. B. et al. (2018) Nature Biotechnology 36(1): 70-80); SNES (see e.g., Leung M. L. et al. (2015) Genome Biol 16: 55); snmC-Seq (see e.g., Luo C. et al. (2017) Science 357(6351): 600); snRNA-seq (see e.g., Grindberg R. V. et al. (2013) Proc Natl Acad Sci USA 110: 19802-7); SPLiT-seq (see e.g., Rosenberg A. B. et al. (2018) Science 360(6385): 176); STRT (see e.g., Islam S. et al. (2011) Genome Res 21: 1160-1167); SUPeR-seq (see e.g., Fan X. et al. (2015) Genome Biol 16: 148); TCR Chain Pairing (see e.g., Turchaninova M. A. et al. (2013) Eur J Immunol 43: 507-2515); TCR-LA-MC-PCR (see e.g., Ruggiero E. et al. (2015) Nat Commun 6: 8081); TIVA (see e.g., Lovatt D. et al. (2014) Nat Methods 11: 190-196); TSCS (see e.g., Casasent A. K. et al. (2018) Cell 172(1): 205-217.e212); UMI Method (see e.g., Kivioja T. et al. (2012) Nat Methods 9: 72-74); and viscRNA-seq (see e.g., Zanini F. et al. (2018) Elife 7: e32942).

In certain embodiments, the invention involves single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p 666-673, 2012).

In certain embodiments, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain embodiments, the invention involves high-throughput single-cell RNA-seq. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. Jan; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; Gierahn et al., “Seq-Well: portable, low-cost RNA sequencing of single cells at high throughput” Nature Methods 14, 395-398 (2017); and Hughes, et al., “Highly Efficient, Massively-Parallel Single-Cell RNA-Seq Reveals Cellular States and Molecular Features of Human Skin Pathology” bioRxiv 689273; doi: doi.org/10.1101/689273, all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; International patent application number PCT/US2016/059239, published as WO2017164936 on Sep. 28, 2017; International patent application number PCT/US2018/060860, published as WO/2019/094984 on May 16, 2019; International patent application number PCT/US2019/055894, published as WO/2020/077236 on Apr. 16, 2020; and Drokhlyansky, et al., “The enteric nervous system of the human and mouse colon at a single-cell resolution,” bioRxiv 746743; doi: doi.org/10.1101/746743, which are herein incorporated by reference in their entirety.

In certain embodiments, the invention involves the Assay for Transposase Accessible Chromatin using sequencing (ATAC-seq) as described. (see, e.g., Buenrostro, et al., Transposition of native chromatin for fast and sensitive epigenomic profiling of open chromatin, DNA-binding proteins and nucleosome position. Nature methods 2013; 10 (12): 1213-1218; Buenrostro et al., Single-cell chromatin accessibility reveals principles of regulatory variation. Nature 523, 486-490 (2015); Cusanovich, D. A., Daza, R., Adey, A., Pliner, H., Christiansen, L., Gunderson, K. L., Steemers, F. J., Trapnell, C. & Shendure, J. Multiplex single-cell profiling of chromatin accessibility by combinatorial cellular indexing. Science. 2015 May 22; 348(6237):910-4. doi: 10.1126/science.aab1601. Epub 2015 May 7; US20160208323A1; US20160060691A1; and WO2017156336A1).

### CRISPR-Effector System-Based Nucleic Acid Detection

In some embodiments, polynucleotide detection can include detection by a CRISPR-Effector system, such as any such system having collateral activity. In some embodiments, the CRISPR-Effector system can include a gRNA capable of binding a target polynucleotide and a Cas effector. In some embodiments, the Cas effector can have collateral polynucleotide activity and can be used to detect a replication-specific feature described herein. The CIRSPR-effector system or component thereof can be included in a composition with one or more other reagents (including but not limited to an amplification reagent), molecules, etc. to facilitate detection and/or measuring of one or more effects of the pool of target compounds. Such systems are also referred to as CRISPR diagnostics and can be configured to detect specific DNAs and RNAs and provide a detectable signal upon detection by capitalizing on the collateral effect of the CRISPR system. Such systems are described in e.g., Vangah et al Biol Proced Online. 2020. 22:22 doi: 10.1186/s12575-020-00135-3, Patchsung et al. 2020. Nat Biomed Eng. August 26. doi: 10.1038/s41551-020-00603-x; Barnes et al. 2020 Nat. Commun. 11(1):4131; Iwasaki and Batey. 2020. Nuc. Acid Res. 2020 Sep. 25; 48(17):e101. doi: 10.1093/nar/gkaa673; Joung et al. 2020. medRxiv. 2020 May 8:2020.05.04.20091231. doi: 10.1101/2020.05.04.20091231; de Puig et al. Annu Rev Biomed Eng. 2020 Jun. 4; 22:371-386. doi: 10.1146/annurev-bioeng-060418-052240; Baerwald et al. 2020, Mol Ecol Resour. 2020 July; 20(4):961-970. doi: 10.1111/1755-0998.13186; Ackerman et al., Nature. 2020 June; 582(7811):277-282. doi: 10.1038/s41586-020-2279-8; Petri and Pattanayak et al., CRISPR J. 2018 June; 1:209-211. doi: 10.1089/crispr.2018.29018.kpe; Batista and Pacheco et al., J Microbiol Methods. 2018 September; 152:98-104. doi: 10.1016/j.mimet.2018.07.024; Gootenberg et al. Science. 2018 Apr. 27; 360(6387):439-444. doi: 10.1126/science.aaq0179; Gootenberg et al. Science. 2017 Apr. 28; 356(6336):438-442. doi: 10.1126/science.aam9321; PCT/US18/054472 filed Oct. 22, 2018 at [0183]-[0327], incorporated herein by reference. Reference is made to WO 2017/219027, WO2018/107129, US20180298445, US 2018-0274017, US 2018-0305773, WO 2018/170340, U.S. application Ser. No. 15/922,837, filed Mar. 15, 2018 entitled “Devices for CRISPR Effector System Based Diagnostics”, PCT/US18/50091, filed Sep. 7, 2018 “Multi-Effector CRISPR Based Diagnostic Systems”, PCT/US18/66940 filed Dec. 20, 2018 entitled “CRISPR Effector System Based Multiplex Diagnostics”, PCT/US18/054472 filed Oct. 4, 2018 entitled “CRISPR Effector System Based Diagnostic”, U.S. Provisional 62/740,728 filed Oct. 3, 2018 entitled “CRISPR Effector System Based Diagnostics for Hemorrhagic Fever Detection”, U.S. Provisional 62/690,278 filed Jun. 26, 2018 and U.S. Provisional 62/767,059 filed Nov. 14, 2018 both entitled “CRISPR Double Nickase Based Amplification, Compositions, Systems and Methods”, U.S. Provisional 62/690,160 filed Jun. 26, 2018 and U.S. Pat. No. 62,767,077 filed Nov. 14, 2018, both entitled “CRISPR/CAS and Transposase Based Amplification Compositions, Systems, And Methods”, U.S. Provisional 62/690,257 filed Jun. 26, 2018 and 62/767,052 filed Nov. 14, 2018 both entitled “CRISPR Effector System Based Amplification Methods, Systems, And Diagnostics”, U.S. Provisional 62/767,076 filed Nov. 14, 2018 entitled “Multiplexing Highly Evolving Viral Variants With SHERLOCK” and 62/767,070 filed Nov. 14, 2018 entitled “Droplet SHERLOCK.” Reference is further made to WO2017/127807, WO2017/184786, WO 2017/184768, WO 2017/189308, WO 2018/035388, WO 2018/170333, WO 2018/191388, WO 2018/213708, WO 2019/005866, PCT/US18/67328 filed Dec. 21, 2018 entitled “Novel CRISPR Enzymes and Systems”, PCT/US18/67225 filed Dec. 21, 2018 entitled “Novel CRISPR Enzymes and Systems” and PCT/US18/67307 filed Dec. 21, 2018 entitled “Novel CRISPR Enzymes and Systems”, U.S. 62/712,809 filed Jul. 31, 2018 entitled “Novel CRISPR Enzymes and Systems”, U.S. 62/744,080 filed Oct. 10, 2018 entitled “Novel Cas12b Enzymes and Systems” and U.S. 62/751,196 filed Oct. 26 2018 entitled “Novel Cas12b Enzymes and Systems”, U.S. 715,640 filed August 7, 2-18 entitled “Novel CRISPR Enzymes and Systems”, WO 2016/205711, U.S. Pat. No. 9,790,490, WO 2016/205749, WO 2016/205764, WO 2017/070605, WO 2017/106657, and WO 2016/149661, WO2018/035387, WO2018/194963, Cox DBT, et al., RNA editing with CRISPR-Cas13, Science. 2017 Nov. 24; 358(6366):1019-1027; Gootenberg J S, et al., Multiplexed and portable nucleic acid detection platform with Cas13, Cas12a, and Csm6, Science. 2018 Apr. 27; 360(6387):439-444; Gootenberg J S, et al., Nucleic acid detection with CRISPR-Cas13a/C2c2, Science. 2017 Apr. 28; 356(6336):438-442; Abudayyeh O O, et al., RNA targeting with CRISPR-Cas13, Nature. 2017 Oct. 12; 550(7675):280-284; Smargon A A, et al., Cas13b Is a Type VI-B CRISPR-Associated RNA-Guided RNase Differentially Regulated by Accessory Proteins Csx27 and Csx28. Mol Cell. 2017 Feb. 16; 65(4):618-630.e7; Abudayyeh O O, et al., C2c2 is a single-component programmable RNA-guided RNA-targeting CRISPR effector, Science. 2016 Aug. 5; 353(6299):aaf5573; Yang L, et al., Engineering and optimising deaminase fusions for genome editing. Nat Commun. 2016 Nov. 2; 7:13330, Myrvhold et al., Field deployable viral diagnostics using CRISPR-Cas13, Science 2018 360, 444-448, Shmakov et al. “Diversity and evolution of class 2 CRISPR-Cas systems,” Nat Rev Microbiol. 2017 15(3):169-182, each of which is incorporated herein by reference in its entirety and can be adapted for use with the methods described herein.

### Amplification of Nucleic Acids

In some embodiments, the method of analyzing an effect includes amplification of a polynucleotide from the subject. Any suitable RNA or DNA amplification technique may be used. In certain example embodiments, the RNA or DNA amplification is an isothermal amplification. In certain example embodiments, the isothermal amplification may be nucleic-acid sequenced-based amplification (NASBA), recombinase polymerase amplification (RPA), loop-mediated isothermal amplification (LAMP), strand displacement amplification (SDA), helicase-dependent amplification (HDA), or nicking enzyme amplification reaction (NEAR). In certain example embodiments, non-isothermal amplification methods may be used which include, but are not limited to, PCR, multiple displacement amplification (MDA), rolling circle amplification (RCA), ligase chain reaction (LCR), or ramification amplification method (RAM). In certain embodiments, the amplification can utilize a transposase-based isothermal amplification method (see e.g. WO 2020/006049, which is incorporated by reference herein as if expressed in its entirety), nickase-based isothermal amplification method (see e.g. WO 2020/006067, which is incorporated by reference herein as if expressed in its entirety), a helicase-based amplification method (see e.g. WO 2020/006036, which is incorporated by reference herein as if expressed in its entirety), polymerase chain reaction (PCR), quantitative real-time PCR; reverse transcriptase PCR (RT-PCR); real-time PCR (rt PCR); real-time reverse transcriptase PCR (rt RT-PCR); nested PCR; strand displacement amplification; transcription-free isothermal amplification; ligase chain reaction amplification; gap filling ligase chain reaction amplification; coupled ligase detection and PCR; or other methods known in the art. In some embodiments, amplification is via LAMP. In some embodiments, amplification is via RPA.

In certain example embodiments, the RNA or DNA amplification is nucleic acid sequence-based amplification is NASBA, which is initiated with reverse transcription of target RNA by a sequence-specific reverse primer to create a RNA/DNA duplex. RNase H is then used to degrade the RNA template, allowing a forward primer containing a promoter, such as the T7 promoter, to bind and initiate elongation of the complementary strand, generating a double-stranded DNA product.

In certain other example embodiments, a recombinase polymerase amplification (RPA) reaction may be used to amplify the target nucleic acids. RPA reactions employ recombinases which are capable of pairing sequence-specific primers with homologous sequence in duplex DNA. If target DNA is present, DNA amplification is initiated and no other sample manipulation such as thermal cycling or chemical melting is required. The entire RPA amplification system is stable as a dried formulation and can be transported safely without refrigeration. RPA reactions may also be carried out at isothermal temperatures with an optimum reaction temperature of 37-42° C. The sequence specific primers are designed to amplify a sequence comprising the target nucleic acid sequence to be detected. In certain example embodiments, a RNA polymerase promoter, such as a T7 promoter, is added to one of the primers. This results in an amplified double-stranded DNA product comprising the target sequence and a RNA polymerase promoter. After, or during, the RPA reaction, a RNA polymerase is added that will produce RNA from the double-stranded DNA templates.

Accordingly, in certain example embodiments the systems disclosed herein may include amplification reagents. Different components or reagents useful for amplification of nucleic acids are described herein. For example, an amplification reagent as described herein may include a buffer, such as a Tris buffer. A Tris buffer may be used at any concentration appropriate for the desired application or use, for example including, but not limited to, a concentration of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 25 mM, 50 mM, 75 mM, 1 M, or the like. One of skill in the art will be able to determine an appropriate concentration of a buffer such as Tris for use with the present invention.

A salt, such as magnesium chloride (MgCl₂), potassium chloride (KCl), or sodium chloride (NaCl), may be included in an amplification reaction, such as PCR, in order to improve the amplification of nucleic acid fragments. Although the salt concentration will depend on the particular reaction and application, in some embodiments, nucleic acid fragments of a particular size may produce optimum results at particular salt concentrations. Larger products may require altered salt concentrations, typically lower salt, in order to produce desired results, while amplification of smaller products may produce better results at higher salt concentrations. One of skill in the art will understand that the presence and/or concentration of a salt, along with alteration of salt concentrations, may alter the stringency of a biological or chemical reaction, and therefore any salt may be used that provides the appropriate conditions for a reaction of the present invention and as described herein.

Amplification reactions may include dNTPs and nucleic acid primers used at any concentration appropriate for the invention, such as including, but not limited to, a concentration of 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or the like.

### Other Nucleic Acid Analysis Techniques

Other techniques can be used to analyze nucleic acids to determine one or more effects of the pool of target compounds on the subject. These include without limitation, immunofluorescence, immunohistochemistry, fluorescence activated cell sorting (FACS), mass cytometry (CyTOF), MERFISH (multiplex (in situ) RNA FISH) and/or by in situ hybridization. Other methods including absorbance assays and colorimetric assays are known in the art and may be used herein.

### PCR-Based Polynucleotide Detection

In some embodiments, a PCR-based polynucleotide detection can be used detect or measure an effect of the pool of test compounds. In some embodiments, the PCR-based detection method selectively amplifies the target molecule, thus providing specific detection of the target molecule. Some techniques involve direct amplification of the polynucleotide. Other techniques involve amplification of a proxy for the original target molecule such as cDNA or cRNA. Exemplary PCR-based polynucleotide detection methods include, without limitation, semi-qualitative, semi-quantitative, or quantitative PCR, quantitative real-time PCR, reverse transcriptase PCR, real-time reverse transcriptase PCR (rt RT-PCR), nested PCR, strand displacement amplification (see U.S. Pat. No. 5,744,311); transcription-free isothermal amplification (see U.S. Pat. No. 6,033,881); repair chain reaction amplification (see WO 90/01069); ligase chain reaction amplification (see EP-A-320 308); gap filling ligase chain reaction amplification (see U.S. Pat. No. 5,427,930); coupled ligase detection and PCR (see U.S. Pat. No. 6,027,889); and NASBATM RNA transcription-free amplification (see U.S. Pat. No. 6,025,134).

### Epigenome Analysis Techniques

As used herein the term “Epigenome, Epigenetics” and the like refer to changes, which can be heritable, in gene activity caused by something other than DNA (or genome) sequence changes, and include without limitation, DNA methylation, DNA-protein interactions, chromatin accessibility, and histone isoforms, modifications, and location (occupancy) in genome regions. In some embodiments, the method includes performing a technique to detect one or more of an epigenetic change in response to the pool of target compounds in the subject. In some embodiments, a sequencing- and/or an array-based technique is used to analyze DNA methylation and include methylation sequencing with a next-generation sequencing technique and the use of methylation microarrays both capable of analyzing the methylation state of various CpGs.

In some cases, the DNA methylation may be detected in a methylation assay utilizing next-generation sequencing. For example, DNA methylation may be detected by massive parallel sequencing with bisulfite conversion, e.g., whole-genome bisulfite sequencing or reduced representation bisulfite sequencing. Optionally, the DNA methylation is detected by microarray, such as a genome-wide microarray. Microarrays, and massively parallel sequencing, have enabled the interrogation of cytosine methylation on a genome-wide scale (Zilberman D, Henikoff S. 2007. Genome-wide analysis of DNA methylation patterns. Development 134(22): 3959-3965.). Genome wide methods have been described previously (Deng, et al. 2009. Targeted bisulfite sequencing reveals changes in DNA methylation associated with nuclear reprogramming. Nat Biotechnol 27(4): 353-360; Meissner, et al. 2005. Reduced representation bisulfite sequencing for comparative high-resolution DNA methylation analysis. Nucleic Acids Res 33(18): 5868-5877; Down, et al. 2008. A Bayesian deconvolution strategy for immunoprecipitation-based DNA methylome analysis. Nat Biotechnol 26(7): 779-785; Gu et al. 2011. Preparation of reduced representation bisulfite sequencing libraries for genome-scale DNA methylation profiling. Nat Protoc 6(4): 468-481).

In some embodiments, DNA methylation may be detected by whole genome bisulfite sequencing (WGBS) (Cokus, et al. 2008. Shotgun bisulphite sequencing of the Arabidopsis genome reveals DNA methylation patterning. Nature 452(7184): 215-219; Lister, et al. 2009. Human DNA methylomes at base resolution show widespread epigenomic differences. Nature 462(7271): 315-322; Harris, et al. 2010. Comparison of sequencing-based methods to profile DNA methylation and identification of monoallelic epigenetic modifications. Nat Biotechnol 28(10): 1097-1105).

In certain cases, DNA methylation may be detected methylation-specific PCR, whole genome bisulfite sequence, the HELP assay and other methods using methylation-sensitive restriction endonucleases, ChiP-on-chip assays, restriction landmark genomic scanning, COBRA, Ms-SNuPE, methylated DNA immunoprecipitation (MeDip), pyrosequencing of bisulfite treated DNA, molecular break light assay for DNA adenine methyltransferase activity, methyl sensitive Southern blotting, methylCpG binding proteins, mass spectrometry, HPLC, and reduced representation bisulfite sequencing. In some embodiments, the DNA methylation is detected in a methylation assay utilizing next-generation sequencing. For example, DNA methylation may be detected by massive parallel sequencing with bisulfite conversion, e.g., whole-genome bisulfite sequencing or reduced representation bisulfite sequencing. Optionally, the DNA methylation is detected by microarray, such as a genome-wide microarray.

A methylation profile can be determined from the methods disclosed herein. In embodiments, determining the methylation profile comprises generating a genome-wide methylation profile of the cells. Neighborhood methylation profile analysis may be performed by analyzing the loci with which any given locus was in contact. Such analysis may be used to evaluate can how the chromatin neighborhood affected the methylation state of the DNA of that locus. Aggregate methylation profile may also be performed to sum the methylation profile at a large number of positions and to reveal subtle effects in WGBS data. In some examples, aggregate methylation analysis may be performed by plotting DNA methylation in the vicinity of selected sequences (e.g., motifs) and compare it to nucleosome occupancy data (e.g., from MNase-Seq). Methylation profile may comprise unmethylation, methylation and co-methylation at each end of the end-joined nucleic acid fragments.

In some embodiments, DNA-protein interactions can be evaluated using a ChIP assay, ChIP-Seq, DNA electrophoretic mobility shift assay, DNA pull down assays, a microplate capture and detection assay, or a reporter assay (such as a Luciferase-based reporter assay). Such assays are generally known in the art.

Histone analysis can include detection of histone isoforms, modifications, and/or location can be analyzed using techniques such as immunodetection assays (e.g. ELISA, Western Blot, ChIP, ChIP-Seq, immunofluorescence, Histone Acetyltransferase assay, histone deacetylase assay, Mitotic assays, mass spectrometry and others), Histone modifications that can be analyzed include, without limitation, acetylation, methylation, phosphorylation, ubiquitylation, glycosylation, ADP-ribosylation, carbonylation and SUMOylation.

The epigenome can also include the presence or level of non-translated RNAs such as RNAi. These can be detected by methods previously described in relation to nucleic acid and transcriptome detection and analysis.

### Protein Analysis

Proteins can be evaluated using a variety of techniques generally known to those of ordinary skill in the art. In some embodiments, the protein analysis includes analyzing the primary, secondary, tertiary, quaternary structure of the protein (or complex as the case may be). In some embodiments, the analysis includes analyzing one or more functionalities of the protein(s). Suitable techniques include, without limitation, protein sequencing (e.g. Edman, de novo, or peptide mass fingerprinting), mass-spectrometry, immunochemical techniques, histological techniques (e.g. staining techniques), immunofluorescent techniques, FACS, post-translation modification analysis (e.g. glycosylation analysis), a light scattering technique (e.g., batch dynamic light scattering, static light scattering, charge and zeta potential determination, circular dichroism spectrometry, isothermal titration calorimetry, size separation technique (e.g. gel electrophoresis), charge-based separation technique (e.g. isoelectric focusing), affinity-based separation technique, X-ray crystallography, SEM crystallography technique, a spatial proteomic technique, and any combination thereof.

### Multi-Omic Analysis

In some embodiments, analysis of one or more effects of the pool of target compounds can include multi-omic analysis. Multi-omic analysis, or simply multiomics, refers to the analytical approach of a biological sample in which the data sets are from multiple “omes”, such as the genome, transcriptome, proteome, epigenome, metabolome, microbiome, and the like. In some embodiments, such multiomic approach can be a single-cell multiomic approach, which includes multilevel single-cell data (such as that obtained from a single-cell genomic data and single cell protein, epigenome transcriptome or other data from (e.g. a spatial proteomic technique (see e.g. proximity extension assays using e.g. DNA barcoded antibodies (see e.g., Assarsson, et al. 2014. “Homogenous 96-Plex PEA Immunoassay Exhibiting High Sensitivity, Specificity, and Excellent Scalability”. PLoS ONE. 9 (4): e95192), mass cytometry for multiomics (see e.g. Gherardini, et al. 2016. “Highly multiplexed simultaneous detection of RNAs and proteins in single cells”. Nature Methods. 13 (3): 269-275. doi:10.1038/nmeth.3742. ISSN 1548-7105), single-cell bisulfite sequencing (see e.g., Kelsey, et al. 2014. “Single-Cell Genome-Wide Bisulfite Sequencing for Assessing Epigenetic heterogeneity”. Nature Methods. 11 (8): 817-820), sc-RNA Seq, scATAC-Seq and scHiC (see e.g., Fraser, et. al. 2013. “Single-cell Hi-C reveals cell-to-cell variability in chromosome structure”. Nature. 502 (7469): 59-64).

### MS Methods

Biomarker detection may also be evaluated using mass spectrometry methods. A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al., Anal. Chem. 70:647 R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS).sup.N, atmospheric pressure photoionization mass spectrometry (APPI-MS), APPI-MS/MS, and APPI-(MS).sup.N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab)₂ fragment, a single chain antibody fragment, an Fv fragment, a single chain Fv fragment, a nucleic acid, a lectin, a ligand-binding receptor, affibodies, nanobodies, ankyrins, domain antibodies, alternative antibody scaffolds (e.g. diabodies etc.) imprinted polymers, avimers, peptidomimetics, peptoids, peptide nucleic acids, threose nucleic acid, a hormone receptor, a cytokine receptor, and synthetic receptors, and modifications and fragments of these.

### Immunoassays

Immunoassay methods are based on the reaction of an antibody to its corresponding target or analyte and can detect the analyte in a sample depending on the specific assay format. To improve specificity and sensitivity of an assay method based on immunoreactivity, monoclonal antibodies are often used because of their specific epitope recognition. Polyclonal antibodies have also been successfully used in various immunoassays because of their increased affinity for the target as compared to monoclonal antibodies Immunoassays have been designed for use with a wide range of biological sample matrices Immunoassay formats have been designed to provide qualitative, semi-quantitative, and quantitative results.

Quantitative results may be generated through the use of a standard curve created with known concentrations of the specific analyte to be detected. The response or signal from an unknown sample is plotted onto the standard curve, and a quantity or value corresponding to the target in the unknown sample is established.

Numerous immunoassay formats have been designed. ELISA or EIA can be quantitative for the detection of an analyte/biomarker. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I¹²⁵) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

### Hybridization Assays

Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of a signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the biomarkers whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acids provides information regarding expression for each of the biomarkers that have been probed, where the expression information is in terms of whether or not the gene is expressed and, typically, at what level, where the expression data, i.e., expression profile, may be both qualitative and quantitative.

Optimal hybridization conditions will depend on the length (e.g., oligomer vs. polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., “Current Protocols in Molecular Biology”, Greene Publishing and Wiley-interscience, NY (1987), which is incorporated in its entirety for all purposes. When the cDNA microarrays are used, typical hybridization conditions are hybridization in 5×SSC plus 0.2% SDS at 65 C for 4 hours followed by washes at 25° C. in low stringency wash buffer (1×SSC plus 0.2% SDS) followed by 10 minutes at 25° C. in high stringency wash buffer (0.1 SSC plus 0.2% SDS) (see Shena et al., Proc. Natl. Acad. Sci. USA, Vol. 93, p. 10614 (1996)). Useful hybridization conditions are also provided in, e.g., Tijessen, Hybridization With Nucleic Acid Probes”, Elsevier Science Publishers B.V. (1993) and Kricka, “Nonisotopic DNA Probe Techniques”, Academic Press, San Diego, Calif. (1992).

### Cell Lysis

In some embodiments, the method includes lysing one or more cells. Cell lysis can be achieved by any suitable physical or chemical method. Suitable physical methods include, but are not limited to, thermal (repeated freeze-thaw cycles), sonic (e.g. ultrasound), electroporation, and physical bombardment. Chemical cell lysis can be achieved by exposing the cell(s) to chemicals that disrupt the cell wall and/or cell membrane. Such chemicals include, without limitation, detergents, acids, bases, salts and others that will be appreciated by those of ordinary skill in the art. Exemplary salts include, without limitation, NaCl, KCl, ammonium sulfate [(NH₄)₂SO₄], or others. Detergents that may be appropriate for the invention may include Triton X-100, sodium dodecyl sulfate (SDS), CHAPS (3-[(3-cholamidopropyl)dimethylammonio]-1-propanesulfonate), ethyl trimethyl ammonium bromide, nonyl phenoxypolyethoxylethanol (NP-40). Concentrations of detergents may depend on the particular application and may be specific to the reaction in some cases.

In some embodiments, such as single-cell sequencing techniques, lysing can occur in a bead. In certain example embodiments, sequencing DNA or other polynucleotide for each cell comprises lysing the cell in each bead such that genomic DNA is retained in the polymerized bead.

### High-Throughput Screening of Test Compounds

The pools of target compounds described in greater detail elsewhere herein can be used for the high-throughput screening of test compounds so as to identify, for example, therapeutic agents for treating diseases or a symptom thereof, those agents capable of achieving a particular function (such as guiding cell differentiation, evaluating single and multi-compound toxicities, or achieving a beneficial effect (e.g. stimulating hair growth, increasing muscle mass, increase growth rate, decreasing fat, etc.) that may or may not be associated with a disease state. It will be appreciated by those of skill in the art effects measured in the different instances may however, be different, as they will be reflective of the outcome being measured. In some embodiments, the in-parallel is defined as performing a task for multiple subjects simultaneously. In-parallel screening of test compounds, as the phrase is used herein, means that a plurality of test compounds are to be screened together simultaneously.

The number of test compounds to be in-parallel screened can be any number between two and one hundred, one thousand, ten thousand, one hundred thousand, one million, one billion or more. In some embodiments, the number of test compounds is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000, 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 46000, 47000, 48000, 49000, 50000, 51000, 52000, 53000, 54000, 55000, 56000, 57000, 58000, 59000, 60000, 61000, 62000, 63000, 64000, 65000, 66000, 67000, 68000, 69000, 70000, 71000, 72000, 73000, 74000, 75000, 76000, 77000, 78000, 79000, 80000, 81000, 82000, 83000, 84000, 85000, 86000, 87000, 88000, 89000, 90000, 91000, 92000, 93000, 94000, 95000, 96000, 97000, 98000, 99000, 100000, 10000, 120000, 130000, 140000, 150000, 160000, 170000, 180000, 190000, 200000, 210000, 220000, 230000, 240000, 250000, 260000, 270000, 280000, 290000, 300000, 310000, 320000, 330000, 340000, 350000, 360000, 370000, 380000, 390000, 400000, 410000, 420000, 430000, 440000, 450000, 460000, 470000, 480000, 490000, 500000, 510000, 520000, 530000, 540000, 550000, 560000, 570000, 580000, 590000, 600000, 610000, 620000, 630000, 640000, 650000, 660000, 670000, 680000, 690000, 700000, 710000, 720000, 730000, 740000, 750000, 760000, 770000, 780000, 790000, 800000, 810000, 820000, 830000, 840000, 850000, 860000, 870000, 880000, 890000, 900000, 910000, 920000, 930000, 940000, 950000, 960000, 970000, 980000, 990000, 1000000, or more.

In some embodiments, the method of high-throughput in-parallel screening for therapeutic agents (or other active and/or functional agents) disclosed herein comprises contacting a cell, cell population, or a tissue with a pool of test compounds in parallel. In some embodiments, a cell used as test subject for screening can be a stem cell, a cancer cell, a primary cultured cell, a cell line, a cell derived from an animal or a human, a cell with genetic modifications, a cell cultured in attachment manner, or a cell cultured in suspension manner. In some embodiments, the cell is a stem cell. In some embodiments, the stem cell is derived from tissues comprising the intestine, liver, spleen, pancreas, stomach, esophageal, skin, eye, central nervous system, peripheral nervous system, kidney, ovary, breast, testis, uterus, bone, cartilage, bone marrow, peripheral blood, lymph node, thymus, and the lung. In some embodiments, the tissue used as test subject for screening can be an organoid, a biopsy from an animal or a human, a dissected tissue from an animal or a human. The tissue used herein comprises the intestine, liver, spleen, pancreas, stomach, esophageal, skin, eye, central nervous system, peripheral nervous system, kidney, ovary, breast, testis, uterus, bone, cartilage, bone marrow, peripheral blood, lymph node, thymus, and the lung.

In some embodiments, the test subject for screening is an organoid. The organoid can be derived from stem cells from a variety of tissues or organs. The source of stem cells comprises the intestine, liver, spleen, pancreas, stomach, esophageal, skin, eye, central nervous system, peripheral nervous system, kidney, ovary, breast, testis, uterus, bone, cartilage, bone marrow, peripheral blood, lymph node, thymus, and the lung.

In some embodiments, a plurality of test compounds to be screened together in parallel are optimized using the system and methods disclosed herein as described in above paragraphs.

The effect of the pool of test compounds on the subject can be evaluated by detecting and/or measuring a genomic, transcriptomic, epigenomic, proteomic, metabolomic, secretomic, microbiomic, microbiomic, multiomic, “other omic” change (or lack thereof) in response to one or more test compound(s) in the pool. In some embodiments, the effect is measured and/or detected by evaluating a phenotype and/or genotype of the subject. In some embodiments, the effect is measured and/or detected by evaluating a cell state and/or type of the subject. Suitable methods for evaluating the effect(s) of the pool of test compounds are described in greater detail elsewhere herein.

In some embodiments, the effects of a plurality of test compounds on the test subject can be evaluated based on the changes in a signature (such as a gene transcriptional profile (also referred to herein as a transcription or gene signature), genome, epigenome, genetics, epigenetics, metabolome, proteome, and any other biological functions. In some embodiments, the effects of a plurality of test compounds on a cell or a tissue are measured at single-cell level. In some embodiments, one or more biomarkers using one or more analytic techniques or approaches are evaluated in response to exposure of the subject to the plurality of test compounds. Exemplary biomarkers include, but are not limited to, polynucleotides, polypeptides, lipids, carbohydrates, and the like. In some embodiments, the biomarker(s) is/are differentiation (CD) molecules comprising CD2, CD3e, CD4, CD5, CD9, CD10/Neprilysin, CD13/Aminopeptidase N, CD14, CD16a/Fc gamma RIIIA, CD16b/Fc gamma RIIIB, CD18/Integrin beta 2, CD19, CD20/MS4A1, CD22/Siglec-2, CD29, CD33/Siglec-3, CD34, CD36/SR-B3, CD38, CD44, CD45, CD48/SLAMF2, CD49e/Integrin alpha 5, CD54/ICAM-1, CD56/NCAM-1, CD59, CD61/Integrin beta 3, CD62L/L-Selectin, CD66a/CEACAM-1, CD66b/CEACAM-8, CD66c/CEACAM-6, CD66d/CEACAM-3, CD66e/CEACAM-5, CD66f/PSG-1, CD68/SR-D1, CD69, CD90/Thyl, CD93/Clq R1, CD105/Endoglin, CD106/VCAM-1, CD107a/LAMP-1, CD117/c-kit, CD123/IL-3R alpha, CD127/IL-7 R alpha, CD135/Flt-3, CD146/MCAM, CD150/SLAM, CD163, CD164, CD166/ALCAM, CD183/CXCR3, CD192/CCR2, CD200, CD235a/Glycophorin A, CD244/2B4, CD318/CDCP1, and CD33.

In some embodiments, the effects of a plurality of test compounds on a tissue and/or an organoid or organoids are measured at a bulk population level. In some embodiments, the effects of a plurality of test compounds on a cell, cell population, tissue, and/or organoid are measured on the single cell level. In some embodiments, a massive-parallel single-cell RNA sequencing is performed to measure the changes of transcriptome of single cells.

In some embodiments, a multi-omic analytic approach is used to evaluate the effects of a plurality of test compounds on a cell, cell population, tissue, and/or organoid. In some embodiments, the multi-omic analytic approach can include analyzing two or more of the following: the genome, transcriptome, epigenome, proteome, metabolome, secretome, or any combination thereof. In some embodiments, these single cells with measured transcriptome are also identified using other biomarkers comprising cell surface biomarkers, intracellular biomarkers, nuclear biomarkers. These biomarkers can be a protein or proteins, a carbohydrate or carbohydrates, a lipid or lipids, any metabolites, or any derivatives thereof and any combinations thereof. In some embodiments, these single cells with measure transcriptome are identified based on the expression of cluster of differentiation (CD) molecules comprising CD2, CD3e, CD4, CD5, CD9, CD10/Neprilysin, CD13/Aminopeptidase N, CD14, CD16a/Fc gamma RIIIA, CD16b/Fc gamma RIIIB, CD18/Integrin beta 2, CD19, CD20/MS4A1, CD22/Siglec-2, CD29, CD33/Siglec-3, CD34, CD36/SR-B3, CD38, CD44, CD45, CD48/SLAMF2, CD49e/Integrin alpha 5, CD54/ICAM-1, CD56/NCAM-1, CD59, CD61/Integrin beta 3, CD62L/L-Selectin, CD66a/CEACAM-1, CD66b/CEACAM-8, CD66c/CEACAM-6, CD66d/CEACAM-3, CD66e/CEACAM-5, CD66f/PSG-1, CD68/SR-D1, CD69, CD90/Thy1, CD93/C1q R1, CD105/Endoglin, CD106/VCAM-1, CD107a/LAMP-1, CD117/c-kit, CD123/IL-3R alpha, CD127/IL-7 R alpha, CD135/Flt-3, CD146/MCAM, CD150/SLAM, CD163, CD164, CD166/ALCAM, CD183/CXCR3, CD192/CCR2, CD200, CD235a/Glycophorin A, CD244/2B4, CD318/CDCP1, and CD33. In some embodiments, the combination of cellular biomarker or biomarkers with the transcriptional profile of single cells at single-cell level produces a comprehensive identity of the cells under investigation.

In some embodiments, the effect of each test compound in a given pool of test compounds under evaluation is deciphered based on the gene transcriptional profile of each cell and comparison to reference gene transcriptional profile of cells under known treatments. In some embodiments, such reference transcriptional profile is called biological connectivity map. In some embodiments, an example of such biological connectivity map is a CMap as described in Subramanian et al., 2017, Cell 171, 1437-1452. In some embodiments, such reference transcriptional profile can be obtained from public database, such as the GEO datasets curated by the NIH (ncbi.nlm.nih.gov/gds).

### Screening for Compounds Capable of Guiding Cellular Differentiation

Described in certain example embodiments are methods of screening test compounds for those capable of guiding cellular differentiation. Cellular differentiation is the process in which a cell changes from one cell type to another and involves changes in the expression and/or activity of various biological pathways. Identification of compounds capable of stimulating such changes (or inhibiting them) can be useful in guiding cellular differentiation. Guiding cellular differentiation can be useful in various fields, such as bioengineering, as well as in the treatment of diseases. In some embodiments, methods for in vitro evaluation of test compounds capable of guiding stem cell differentiation are disclosed.

In some embodiments, the methods used are a high throughput method as described in greater detail elsewhere herein. In some embodiments, a plurality (e.g. a pool) of test compounds can be evaluated in parallel, and guided differentiation of stem cells can be assessed using scRNA-seq.

In some embodiments, an in vitro cultured organoid is used for these methods. The use of organoid in high-throughput screening provides a number of advantages, including but not limited to providing a scale beyond animal models, providing a profiling complex and dynamic system, and empowering direct comparisons to the tissue of origin in vivo. Other advantages of using organoids for drug screening include that organoids provide a cost-efficient, massive expansion of stem-cell enriched precursors that allow manipulation of model representation (e.g. cell types) and creation of bio-repository.

In some embodiments, the organoid used herein can be derived from gastric, intestinal (for example, small intestinal, colonic, rectum, duodenum or ileum), pancreatic, prostate, lung, breast, kidney, blood vessel or lymphatic vessel organoids. This typically means that the organoids are derived from gastric, intestinal (for example, small intestinal, colonic, rectum, duodenum or ileum), pancreatic, prostate, liver, pancreas, brain, ovary, uterus, skin, esophagus, lung, spleen, kidney, bone, bone marrow, brain, central nervous system, peripheral nervous system, blood, cartilage, fat, and eye lung, breast, kidney, blood vessel or lymphatic vessel cells respectively. In a preferred embodiment, the organoids used for the screening of a plurality of test compounds herein are derived from the intestine of mammals comprising humans and mice.

In some embodiments, the organoid used as test subject herein can be cultured in a variety of culture medium(s). Specialized culture medium allows the organoid to be in specific physiological or structural conditions. In a preferred embodiment, the organoid used herein is derived from the intestine or mammals, thus containing intestinal stem cells (ISCs). In some embodiments, the organoid can be maintained to be an ISC-enriched status by culturing in media comprising EGF, IGF-1, FGF-2, Noggin, DMI-1-1, R-spondin 1, and R-spondin 3. In some embodiments, the concentration of EGF can be 1, 10, 20, 30, 40, 50, 60, or higher than 60 ng/mL. In some embodiments, Noggin can be at a concentration of 10, 20, 30, 40, 50, 60, 70, 80, 90, 100, or higher than 100 ng/mL. In some embodiments, DMH-1 can be at a concentration of 1, 2, 3, 4, 5, 6, 7, 8, 9, 10, or higher than 10 μM. In some embodiments, R-spondin 1 can be used at a concentration of 100, 200, 300, 400, 500, or higher than 500 ng/mL. In some embodiments, R-spondin 3 can be used at a concentration of 10, 50, 100, 200, 250, 300, 400, 500, or higher than 500 ng/mL. In a preferred embodiment, the medium further contains small molecule CHIR 99021 at a concentration of 1, 2, 3, or higher than 3 μM. In a preferred embodiment, the medium further contains small molecule Ly2090314 at a concentration of 1, 2, 3, 4 or higher than 4 nM. In a preferred embodiment, the medium further contains valproic acid at a concentration of 100, 200, 300, 400, 500, 600, 700, 800, 900, 1000, or more 1000 μM. In some embodiments, the organoid can be maintained to be an ISC-enriched status by using different culture media comprising different growth factors.

In some embodiments, the organoid derived from ICSs used as test subject herein can be cultured in a specialized culture medium that contains MPAK agonist, BMP/TGF-b inhibitor, gastrin, and Wnt-axis agonist.

In some embodiments, a defined culture medium is used to drive the guided differentiation of ICSs in organoid. In a preferred embodiment, a defined culture medium for driving differentiation of ICSs into Paneth cells comprises CHIR and DAPT, a gamma-secretase inhibitor to inhibit Notch pathway. In some embodiments, the phenotype and genotype of Paneth cells obtained in such way are to be used as references for evaluation of ability of test compounds to guide ICSs differentiation into Paneth cells. In some embodiments, scRNA-seq is performed for single cells before and after the guiding differentiation of ICSs into Paneth cells.

In some embodiments, a scRNA-seq dataset was also built for primary tissues and organoids that to be bio-banked at single-cell level. These datasets can be used as references to evaluate the similarity between an in vitro cultured organoid and an in vivo intestine tissue at both single-cell level and molecule level. This empowers the approach disclosed in the present invention in pinpointing the target molecule(s) and target cell(s).

In some embodiments, the primary tissues comprise ileum, duodenum, colon, and rectum from human, mouse, or any other animals. In some embodiments, a plurality of test compounds in a given pool are to be added to an organoid culture, and the changes in phenotype and transcriptional profile of single cells of the organoid are determined. In some embodiments, the changes in genomics, epigenomics, proteomics, metabolomics, genetics, and epigenetics are also determined in single cells or whole organoid before and after the treatment with a plurality of test compounds.

In some embodiments, the differentiation of ICSs into functional mature cells comprises Paneth cells, goblet cells, enteroendocrine cells, M cells, Tuft cells, and enterocytes. In a preferred embodiment, test compounds capable of guiding differentiation of ICSs into these cells are identified based on single-cell transcriptomes measured before and after treatments.

### Screening for Disease Diagnosis, Prognosis, Staging, and Monitoring

Evaluating a response to one or more compounds can provide valuable information on cell state and thus can allow for disease diagnosis, prognosis, staging, and monitoring disease progression or regression and/or tolerance or efficacy of a treatment. Described herein are methods of screening to diagnose, prognose, and/or monitor a disease. Such methods can include a high-throughput method and/or pool of test compounds as previously described.

As used herein, “diagnosing” encompasses detecting, analyzing, measuring, and/or determining the existence, nature, stage, and/or characteristic of a disease, disorder, condition, syndrome, or a symptom thereof in a subject. As understood by those skilled in the art, a diagnosis does not necessarily indicate that it is certain that a subject certainly has the disease, but rather that it is very likely that the subject has the disease. It will be appreciated that in some cases, the diagnosis is a certainty that a subject has a particular disease, disorder, condition, syndrome, or a symptom thereof. A diagnosis can be provided with varying levels of certainty, such as indicating that the presence of the disease is 90% likely, 95% likely, 98%, 99%, or 100% likely, for example. The term diagnosis, as used herein also encompasses determining the severity and probable outcome of disease or episode of disease or prospect of recovery, which is generally referred to as prognosis. The term diagnosis, as used herein, also encompasses determining a stage and/or other characteristic of a disease.

As used herein, “prognosis”, “prognose”, or “prognosing” refer to a prediction of a probability, course, or outcome. Specifically, “prognosing a disease” refers to the prediction that a subject has the disease or a symptom thereof or that a subject will develop a mitochondrial disease or a symptom thereof. For example, the prognostic methods of the instant invention provide for determining whether a sample from a subject exhibits specific characteristics (e.g. a specific response to one or more test agents and/or disease characteristic (discussed elsewhere herein), which can be used to predict whether a subject in need thereof has or will develop a disease or a symptom thereof.

As used herein, “monitoring” refers to evaluating the development (or non-development) and/or progression (or non-progression or regression) of a disease or a symptom thereof or an indicator (e.g. a biomarker, signature, effect of one or more test agents on a biological sample, and the like) in a subject over a period of time. In such embodiments, the methods herein or one or more steps thereof can be performed multiple times on samples obtained over time, where changes in the outcome of the screen can be used to determine the development or progression of a disease or a symptom thereof.

As used herein, “staging” or “staging a disease” refers to evaluating the developmental stage or grade of a disease based on an indicator (e.g. a biomarker, signature, effect of one or more test agents on a biological sample, and the like) measured in a biological sample and/or the subject at a specific point in time. The stage of the disease can be used to classify a subject into a particular patient population and can be used to determine appropriate course of treatment.

As such also described herein are methods of diagnosing, prognosing, monitoring, and/or staging a disease in a subject, that can include contacting, in vitro, a biological sample obtained from the subject with a pool of test compounds in parallel, wherein the test compounds in the pool are selected using any method described elsewhere herein; evaluating the effect of one or more of the test compounds in the pool of test compounds on the biological sample, whereby the specific biological and/or pharmaceutical effects of a test compound are determined and whereby the specific and/or pharmaceutical effect(s) of a test compound determined is/are indicative of a disease, a disease symptom, and/or stage of a disease in the subject.

In some embodiments, evaluating the effect of one or more of the test compounds comprises measuring changes in one or more biologic activities of the biological sample.

In some embodiments, measuring changes in one or more biologic activities comprises a transcript or transcriptome analysis, a gene or genomic analysis, an epigenome analysis, a protein or proteome analysis, a metabolomic analysis, a phenotype analysis, a genetic analysis, or a combination thereof.

In some embodiments, evaluating the effect of one or more of the test compounds further comprises performing deconvolution of the biological and/or pharmacological effects of the pool, wherein the deconvolution comprises computational de-coding using methods comprising large linear computational models.

In some embodiments, the biological sample is a cell or cell population, organoid, or tissue. In some embodiments, the biological sample is a biopsy sample obtained from a subject.

In some embodiments, the biological sample is isolated from, derived from, and/or comprises, consists essentially of, or consists of cells or tissue of the intestine, liver, pancreas, ovary, uterus, skin, esophagus, lung, spleen, kidney, bone, bone marrow, brain, central nervous system, peripheral nervous system, blood, cartilage, fat, eye, heart, lymph node, lymphatic system, thyroid, endocrine system tissue or gland, or a combination thereof.

In some embodiments, the test pool is an optimized test pool. Methods of optimizing the test pool are described in greater detail elsewhere herein.

In some embodiments, the number of test compounds in the test pool is any number from 1 to 1,000,000.

Methods of Determining Cell State and/or Disease Characteristics

The identified effects from one or more of the test compounds in the pool can be used to identify one or more characteristics of a disease, such as a pathway involved in the disease or symptom thereof. In short, if a test compound has an effect on a sample such that the sample changes from a diseased state to a less- or non-diseased state or vice versa (or from one cell state to another) and it is known or identified by the test compound screen what biological process or component thereof the test compound is affecting, then it can be determined that said biological process or component thereof is involved in the disease or symptom thereof (i.e. is a disease characteristic) (or a particular cell state).

As such, also described herein are methods of determining one or more characteristics of a disease or cell state, that can include contacting a biological sample in vitro with a pool of test compounds in parallel, wherein the test compounds in the pool are selected using any one or more of the methods described elsewhere herein; evaluating the effect of one or more of the test compounds in the pool of test compounds on the biological sample, whereby the specific biological and/or pharmaceutical effects of a test compound are determined and whereby the specific and/or pharmaceutical effect(s) of a test compound determined is/are indicative of one or more disease characteristics (or cell state(s)).

In some embodiments, the one or more disease (or cell state) characteristics is determined by evaluating or measuring the expression, activity, or function of one or more genes, proteins, gene programs, biological pathways, cell processes, cell or tissue functions, or combinations thereof.

In some embodiments, evaluating the effect of one or more of the test compounds comprises measuring changes in one or more biologic activities of the biological sample. In some embodiments, measuring changes in one or more biologic activities comprises a transcript or transcriptome analysis, a gene or genomic analysis, an epigenome analysis, a protein or proteome analysis, a metabolomic analysis, a phenotype analysis, a genetic analysis, or a combination thereof.

In some embodiments, evaluating the effect of one or more of the test compounds further comprises performing deconvolution of the biological and/or pharmacological effects of the pool, wherein the deconvolution comprises computational de-coding using methods comprising large linear computational models.

In some embodiments, the biological sample is a cell or cell population, organoid, or tissue.

In some embodiments, the biological sample is a biopsy sample obtained from a subject.

In some embodiments, the biological sample is isolated from, derived from, and/or comprises, consists essentially of, or consists of cells or tissue of the intestine, liver, pancreas, ovary, uterus, skin, esophagus, lung, spleen, kidney, bone, bone marrow, brain, central nervous system, peripheral nervous system, blood, cartilage, fat, eye, heart, lymph node, lymphatic system, thyroid, endocrine system tissue or gland, or a combination thereof.

In some embodiments, the test pool is an optimized test pool. Optimization of the test pool is described in greater detail elsewhere herein.

In some embodiments, the number of test compounds in the test pool is any number from 1 to 1,000,000.

### Therapies

Described in several embodiments herein are therapies for treatment of a disease, disorder or a symptom thereof or achieving a desired effect in a subject. In some embodiments, the treatment includes administering one or more compounds identified in a screen described elsewhere herein to a subject in need of treatment or in need of a desired effect. In some embodiments, the identified compounds can be used to generate a cell, cell population, or tissue that can subsequently be used as a treatment for a disease or disorder in a subject in need thereof.

In some embodiments, the treatment is for disease/disorder of an organ, including brain diseases, liver disease, eye disease, muscle disease, heart disease, blood disease, kidney disease, or may comprise treatment for an autoimmune disease, central nervous system disease, cancer and other proliferative diseases, neurodegenerative disorders, inflammatory disease, metabolic disorder, musculoskeletal disorder and the like.

### Therapies Including the Screen-Identified Compounds

Compounds identified using the screening methods herein can be used a therapies (such as an active primary or secondary agent in a pharmaceutical formulation) for disease or to achieve a particular effect in a subject to be treated. In some embodiments, the particular effect is an effect observed in the screening method used to identify such effective compounds. In some embodiments, the particular effect can be measured in the subject after treatment with one or more compound(s) to, for example, confirm efficacy and/or monitor dosage.

### Pharmaceutical Formulations

Also described herein are pharmaceutical formulations that can contain an amount, effective amount, and/or least effective amount, and/or therapeutically effective amount of one or more compounds and/or compositions identified in a screening method described elsewhere herein (which are also referred to as the primary active agent or ingredient elsewhere herein) described in greater detail elsewhere herein and a pharmaceutically acceptable carrier or excipient. As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo. As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. When present, the compound can optionally be present in the pharmaceutical formulation as a pharmaceutically acceptable salt.

In some embodiments, the active ingredient is present as a pharmaceutically acceptable salt of the active ingredient. As used herein, “pharmaceutically acceptable salt” refers to any acid or base addition salt whose counter-ions are non-toxic to the subject to which they are administered in pharmaceutical doses of the salts. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

The pharmaceutical formulations described herein can be administered to a subject in need thereof via any suitable method or route to a subject in need thereof. Suitable administration routes can include, but are not limited to auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intraci sternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresi s, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the active ingredient(s).

Where appropriate, compounds and/or compositions identified in a screen described elsewhere herein can be provided to a subject in need thereof as an ingredient, such as an active ingredient or agent, in a pharmaceutical formulation. As such, also described are pharmaceutical formulations containing one or more of the compounds and salts thereof, or pharmaceutically acceptable salts thereof described herein. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

In some embodiments, the subject in need thereof has or is suspected of having a disease or a symptom thereof. As used herein, “agent” refers to any substance, compound, molecule, and the like, which can be biologically active or otherwise can induce a biological and/or physiological effect on a subject to which it is administered to. As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed. In some embodiments, “agent” refers to a compound identified in a screening method described elsewhere herein.

### Pharmaceutically Acceptable Carriers and Secondary Ingredients and Agents

The pharmaceutical formulation can include a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In some embodiments, the pharmaceutical formulation can also include an effective amount of secondary active agents, including but not limited to, biologic agents or molecules including, but not limited to, e.g. polynucleotides, amino acids, peptides, polypeptides, antibodies, aptamers, ribozymes, hormones, immunomodulators, antipyretics, anxiolytics, antipsychotics, analgesics, anti spasmodics, anti-inflammatories, anti-hi stamines, anti-infectives, chemotherapeutics, and combinations thereof.

### Effective Amounts

In some embodiments, the amount of the primary active agent and/or optional secondary agent can be an effective amount, least effective amount, and/or therapeutically effective amount. As used herein, “effective amount” refers to the amount of the primary and/or optional secondary agent included in the pharmaceutical formulation that achieve one or more therapeutic effects or desired effect. As used herein, “least effective” amount refers to the lowest amount of the primary and/or optional secondary agent that achieves the one or more therapeutic or other desired effects. As used herein, “therapeutically effective amount” refers to the amount of the primary and/or optional secondary agent included in the pharmaceutical formulation that achieves one or more therapeutic effects. In some embodiments, the one or more effective amount can be the amount that results in an effect observed in a screening method described elsewhere herein.

The effective amount, least effective amount, and/or therapeutically effective amount of the primary and optional secondary active agent described elsewhere herein contained in the pharmaceutical formulation can range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, mg, or g or be any numerical value with any of these ranges.

In some embodiments, the effective amount, least effective amount, and/or therapeutically effective amount can be an effective concentration, least effective concentration, and/or therapeutically effective concentration, which can each range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, μM, mM, or M or be any numerical value with any of these ranges.

In other embodiments, the effective amount, least effective amount, and/or therapeutically effective amount of the primary and optional secondary active agent can range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value with any of these ranges.

In some embodiments, the primary and/or the optional secondary active agent present in the pharmaceutical formulation can range from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation.

In some embodiments where a cell population is present in the pharmaceutical formulation (e.g., as a primary and/or or secondary active agent), the effective amount of cells can range from about 2 cells to 1×10′/mL, 1×10²⁰/mL or more, such as about 1×10′/mL, 1×10²/mL, 1×10³/mL, 1×10⁴/mL, 1×10⁵/mL, 1×10⁶/mL, 1×10⁷/mL, 1×10⁸/mL, 1×10⁹/mL, 1×10¹⁰/mL, 1×10¹¹/mL, 1×10¹²/mL, 1×10¹³/mL, 1×10¹⁴/mL, 1×10¹⁵/mL, 1×10¹⁶/mL, 1×10¹⁷/mL, 1×10¹⁸/mL, 1×10¹⁹/mL, to/or about 1×10²⁰/mL.

In some embodiments, the amount or effective amount, particularly where an infective particle is being delivered (e.g. a virus particle having the primary or secondary agent as a cargo), the effective amount of virus particles can be expressed as a titer (plaque forming units per unit of volume) or as a MOI (multiplicity of infection). In some embodiments, the effective amount can be 1×10¹ particles per pL, nL, μL, mL, or L to 1×10²⁰/particles per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ particles per pL, nL, μL, mL, or L. In some embodiments, the effective titer can be about 1×10¹ transforming units per pL, nL, μL, mL, or L to 1×10²⁰/transforming units per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ transforming units per pL, nL, μL, mL, or L. In some embodiments, the MOI of the pharmaceutical formulation can range from about 0.1 to 10 or more, such as 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 or more.

In some embodiments, the amount or effective amount of the one or more of the active agent(s) described herein contained in the pharmaceutical formulation can range from about 1 pg/kg to about 10 mg/kg based upon the bodyweight of the subject in need thereof or average bodyweight of the specific patient population to which the pharmaceutical formulation can be administered.

In embodiments where there is a secondary agent contained in the pharmaceutical formulation, the effective amount of the secondary active agent will vary depending on the secondary agent, the primary agent, the administration route, subject age, disease, stage of disease, among other things, which will be one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially with the compound, derivative thereof, or pharmaceutical formulation thereof.

In some embodiments, the effective amount of the secondary active agent can range from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total secondary active agent in the pharmaceutical formulation. In additional embodiments, the effective amount of the secondary active agent can range from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation.

### Dosage Forms

In some embodiments, the pharmaceutical formulations described herein can be provided in a dosage form. The dosage form can be administered to a subject in need thereof. The dosage form can be an effective generate specific concentration, such as an effective concentration, at a given site in the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a pharmaceutical formulation thereof calculated to produce the desired response or responses in association with its administration. In some embodiments, the given site is proximal to the administration site. In some embodiments, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In some embodiments, compounds, molecules, compositions, vectors, vector systems, cells, or a combination thereof described herein can be the ingredient whose release is delayed. In some embodiments, the primary active agent is the ingredient whose release is delayed. In some embodiments, an optional secondary agent can be the ingredient whose release is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et al., (Media, Pa.: Williams and Wlkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome. In these embodiments, primary active ingredient(s), and/or optional secondary active ingredient(s), and/or pharmaceutically acceptable salt thereof where appropriate are incorporated into a liposome. In embodiments where the dosage form is a liposome, the pharmaceutical formulation is thus a liposomal formulation. The liposomal formulation can be administered to a subject in need thereof.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, a primary active ingredient, optional secondary active ingredient, and/or pharmaceutically acceptable salt thereof where appropriate can be in a dosage form adapted for inhalation is in a particle-size-reduced form that is obtained or obtainable by micronization. In some embodiments, the particle size of the size reduced (e.g. micronized) compound or salt or solvate thereof, is defined by a D₅₀ value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In some embodiments, the dosage forms are aerosol formulations suitable for administration by inhalation. In some of these embodiments, the aerosol formulation contains a solution or fine suspension of a primary active ingredient, secondary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g. metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional secondary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, 3 or more doses are delivered each time. The aerosol formulations can be administered to a subject in need thereof.

For some dosage forms suitable and/or adapted for inhaled administration, the pharmaceutical formulation is a dry powder inhalable-formulations. In addition to a primary active agent, optional secondary active ingredient, and/or pharmaceutically acceptable salt thereof where appropriate, such a dosage form can contain a powder base such as lactose, glucose, trehalose, mannitol, and/or starch. In some of these embodiments, a primary active agent, secondary active ingredient, and/or pharmaceutically acceptable salt thereof where appropriate is in a particle-size reduced form. In further embodiments, a performance modifier, such as L-leucine or another amino acid, cellobiose oxoacetate, and/or metals salts of stearic acid, such as magnesium or calcium stearate, is provided. In some embodiments, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in some embodiments, from sterile powders, granules, and tablets. The parenteral formulations can be administered to a subject in need thereof.

For some embodiments, the dosage form contains a predetermined amount of a primary active agent, secondary active ingredient, and/or pharmaceutically acceptable salt thereof where appropriate per unit dose. In an embodiment, the predetermined amount of primary active agent, secondary active ingredient, and/or pharmaceutically acceptable salt thereof where appropriate can be an effective amount, a least effect amount, and/or a therapeutically effective amount. In other embodiments, the predetermined amount of a primary active agent, secondary active agent, and/or pharmaceutically acceptable salt thereof where appropriate, can be an appropriate fraction of the effective amount of the active ingredient.

### Co-Therapies and Combination Therapies

In some embodiments, the pharmaceutical formulation(s) described herein can be part of a combination treatment or combination therapy. The combination treatment can include the pharmaceutical formulation described herein and an additional treatment modality. The additional treatment modality can be a chemotherapeutic, a biological therapeutic, surgery, radiation, diet modulation, environmental modulation, a physical activity modulation, and combinations thereof.

In some embodiments, the co-therapy or combination therapy can additionally include but not limited to, polynucleotides, amino acids, peptides, polypeptides, antibodies, aptamers, ribozymes, hormones, immunomodulators, antipyretics, anxiolytics, antipsychotics, analgesics, anti spasmodics, anti-inflammatories, anti-hi stamines, anti-infectives, chemotherapeutics, and combinations thereof.

### Administration of the Pharmaceutical Formulations

The pharmaceutical formulations or dosage forms thereof described herein can be administered one or more times hourly, daily, monthly, or yearly (e.g. 1, 2, 3, 4, 5, 6, 7, 8, 9, 10, 11, 12, 13, 14, 15, 16, 17, 18, 19, 20, or more times hourly, daily, monthly, or yearly). In some embodiments, the pharmaceutical formulations or dosage forms thereof described herein can be administered continuously over a period of time ranging from minutes to hours to days. Devices and dosages forms are known in the art and described herein that are effective to provide continuous administration of the pharmaceutical formulations described herein. In some embodiments, the first one or a few initial amount(s) administered can be a higher dose than subsequent doses. This is typically referred to in the art as a loading dose or doses and a maintenance dose, respectively. In some embodiments, the pharmaceutical formulations can be administered such that the doses over time are tapered (increased or decreased) overtime so as to wean a subject gradually off of a pharmaceutical formulation or gradually introduce a subject to the pharmaceutical formulation.

As previously discussed, the pharmaceutical formulation can contain a predetermined amount of a primary active agent, secondary active agent, and/or pharmaceutically acceptable salt thereof where appropriate. In some of these embodiments, the predetermined amount can be an appropriate fraction of the effective amount of the active ingredient. Such unit doses may therefore be administered once or more than once a day, month, or year (e.g. 1, 2, 3, 4, 5, 6, or more times per day, month, or year). Such pharmaceutical formulations may be prepared by any of the methods well known in the art.

Where co-therapies or multiple pharmaceutical formulations are to be delivered to a subject, the different therapies or formulations can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g. within seconds or just a few minutes apart), where the intent is that the formulations be administered together at the same time.

Therapies Utilizing Cells and/or Tissue Generated Using Guided Differentiation

As previously described, the screening methods can be used to identify one or more compounds that are capable of guided differentiation, particularly of stem cells. In some embodiments, one or more compounds identified in a screen previously described can be used to guide differentiation of one or more cells, such that the one or more cells are changed from a first cell state to a second cell state, wherein the second cell state is a therapeutically effective cell state. As the term is used herein, “therapeutically effective cell state” is the cell state that is capable of providing a therapeutic benefit to a subject when the cell or population thereof is administered to a subject in need thereof. In some embodiments, a tissue or organoid can contain one or more cells having a therapeutically effective cell state. In some embodiments, all of the cells of the organoid or tissue has a therapeutically effective cell state. Such tissues and organoids can also be referred to as therapeutically effective tissues and therapeutically effective organoids, respectively.

In some embodiments, a therapeutically effective tissue can be developed, such as by using one or more compounds identified as being capable of guiding cell differentiation by a screening method described herein, from an organoid. Such a tissue can also be referred to herein as an “organoid-developed” tissue. In some embodiments, the organoid-developed tissue can be used as a treatment, such as to restore the structural and/or physiological function of tissues that the organoid is derived from.

In some embodiments the therapeutically effective cell(s), tissue(s), and/or organoid(s) can be administered to a subject in need thereof. This is a form of adoptive therapy. In some embodiments, the therapeutically effective cell(s), tissue(s), and/or organoid(s) are autologous. In some embodiments, the therapeutically effective cell(s), tissue(s), and/or organoid(s) are heterologous. In some embodiments, the therapeutically effective cell(s), therapeutically effective tissue(s), and/or therapeutically effective organoid(s) to restore an abnormal or diseased tissue to its normal state or non-diseased state. In some embodiments the therapeutically effective cell(s), tissue(s), and/or organoid(s) can produce one or more effective compounds that can be optionally isolated and administered to a subject in need thereof. In some embodiments, the therapeutically effective cell(s), tissue(s), and/or organoid(s) can be developed from, such as by guided differentiation using one or more compounds identified using a screening method described elsewhere herein, from cell(s) and/or tissue obtained from the subject to be treated (i.e. are autologous).

In some embodiments, the treatment is for disease/disorder of an organ, including brain diseases, liver disease, eye disease, muscle disease, heart disease, blood disease, kidney disease, or may comprise treatment for an autoimmune disease, central nervous system disease, cancer and other proliferative diseases, neurodegenerative disorders, inflammatory disease, metabolic disorder, musculoskeletal disorder and the like.

### EXEMPLARY EMBODIMENTS

In one exemplary embodiment, the method can be a method of restoring the function of the barrier structure of the intestine. The intestinal barrier comprises enterocytes, enteroendocrine cells, M cells, Tuft cells, goblet cells, and other types of cells. These cells can be impaired by a variety of pathological conditions including malignant diseases, benign tumors, autoimmune diseases, infectious diseases, environmental and chemical toxin challenges. Thus far, for gastrointestinal diseases, very little attention has been directed to repair the barrier itself to restore the physiological functions and structural barrier.

In some embodiments, the compounds selected using the system and methods disclosed in the present invention for their capability to drive the guided differentiation of IPSs in intestinal organoids represent novel therapeutic approaches to address the pathological conditions aforementioned. Such selected compounds can be small molecules, antibodies, or other types of biological large molecules. These compounds can be administered to a subject in need thereof to act on ISCs in the intestine to drive guided differentiation into desired types of functional cells. In some embodiments, new Paneth cells can be produced in such way. Paneth cells synthesize and secrete substantial quantities of antimicrobial peptides and proteins. These antimicrobial molecules are key mediators of host-microbe interactions, including homeostatic balance with colonizing microbiota and innate immune protection from enteric pathogens. In addition, Paneth cells secrete factors that help sustain and modulate the epithelial stem and progenitor cells that cohabitate in the crypts and rejuvenate the small intestinal epithelium. Thus, Paneth cells play critical roles in immune response and renewal of the intestine. New Paneth cells produced using the methods disclosed herein will help a subject to counter the pathogen challenges, treat infectious diseases, while maintain the ability of self-renewal of intestine epithelium.

In some embodiments, compounds selected using the system and methods disclosed in the present invention for their capability to drive the guided differentiation of pancreatic stem cells in pancreas derived organoids to form functional cells including alpha cells, B cells, delta cells, PP cells, and glandular cells. This therapeutic approach can be used for treating type-I and type-II diabetes, pancreatitis, and pancreatic tumors.

In some embodiments, compounds selected using the system and methods disclosed in the present invention for their capability to drive the guided differentiation of skin stem cells to form skin tissues, keratinocyte sheets, or fibroblast-enriched connective tissues. This therapeutic approach can be used for treating chronic and acute wounds, and for repair of damaged connective tissues such as dura mater.

In some embodiments, the invention disclosed herein relates to compositions and methods for cellular therapy or immunocell therapy. In some embodiments, the newly formed cells from the guided differentiation of stem cells driven by compounds selected using the system and methods disclosed herein can be transplanted into patients in need thereof. In some embodiments, the isolated cell or cells disclosed herein can be modified genetically, epigenetically, genomically, epigenomically, and/or proteomically to have novel or improved functionality and to be transplanted into patients in need thereof.

### Determining Disease Characteristics

The identified effects from the test compounds in the pool can be used to identify one or more characteristics of a disease, such as a pathway involved in the disease or symptom thereof. In short, if a test compound has an effect on a sample such that the sample changes from a diseased state to a less- or non-diseased state or vice versa and it is known or identified by the test compound screen what biological process or component thereof the test compound is affecting, then it can be determined that said biological process or component thereof is involved in the disease or symptom thereof (i.e. is a disease characteristic).

As such, also described herein are methods of determining one or more characteristics of a disease (or cell state) that can include contacting a biological sample in vitro with a pool of test compounds in parallel, wherein the test compounds in the pool are selected using any one or more of the methods described elsewhere herein; evaluating the effect of one or more of the test compounds in the pool of test compounds on the biological sample, whereby the specific biological and/or pharmaceutical effects of a test compound are determined and whereby the specific and/or pharmaceutical effect(s) of a test compound determined is/are indicative of one or more disease characteristics.

In some embodiments, the one or more disease characteristics (or cell states) are determined by evaluating or measuring the expression, activity, and/or function of one or more genes, proteins, gene programs, biological pathways, cell processes, cell or tissue functions, or combinations thereof.

In some embodiments, evaluating the effect of one or more of the test compounds comprises measuring changes in one or more biologic activities of the biological sample. In some embodiments, measuring changes in one or more biologic activities comprises a transcript or transcriptome analysis, a gene or genomic analysis, an epigenome analysis, a protein or proteome analysis, a metabolomic analysis, a phenotype analysis, a genetic analysis, or a combination thereof.

In some embodiments, evaluating the effect of one or more of the test compounds further comprises performing deconvolution of the biological and/or pharmacological effects of the pool, wherein the deconvolution comprises computational de-coding using methods comprising large linear computational models.

In some embodiments, the biological sample is a cell or cell population, organoid, or tissue.

In some embodiments, the biological sample is a biopsy sample obtained from a subject.

In some embodiments, the biological sample is isolated from, derived from, and/or comprises, consists essentially of, or consists of cells or tissue of the intestine, liver, pancreas, ovary, uterus, skin, esophagus, lung, spleen, kidney, bone, bone marrow, brain, central nervous system, peripheral nervous system, blood, cartilage, fat, eye, heart, lymph node, lymphatic system, thyroid, endocrine system tissue or gland, or a combination thereof.

In some embodiments, the test pool is an optimized test pool. Optimization of the test pool is described in greater detail elsewhere herein.

In some embodiments, the number of test compounds in the test pool is any number from 1 to 1,000,000.

Further embodiments are illustrated in the following Examples which are given for illustrative purposes only and are not intended to limit the scope of the invention.

### EXAMPLES

### Example 1. Methods for Crypt Organoid Preparation, Enrichment, and Differentiation

This example describes the methods for preparing crypt organoids. This example also describes the intestinal stem cell (ISC) enrichment and differentiation of the organoids.

Small intestinal crypts were cultured as previously described (see e.g., Sato et al. Nature. 2009; 459:262-265). Briefly, crypts were resuspended in basal culture medium (Advanced DMEM/F12 with 2 mM GlutaMAX and 10 mM HEPES; Thermo Fisher Scientific) at a 1:1 ratio with Corning™ Matrigel™ Membrane Matrix— GFR (Fisher Scientific) and plated at the center of each well of 24-well plates. Following Matrigel polymerization, 500 μL of small intestinal crypt culture medium (basal media plus 100×N2 supplement, 50×B27 supplement; Life Technologies, 500×N-acetyl-L-cysteine; Sigma-Aldrich) supplemented with growth factors EGF (E; 50 ng/mL, Life Technologies), Noggin (N; 100 ng/mL, PeproTech), and R-spondin 1 (R; 500 ng/mL, PeproTech) and small molecules CHIR99021 (C; 3 μM, LC Laboratories) and valproic acid (V; 1 mM, Sigma-Aldrich) was added to each well. ROCK inhibitor Y-27632 (Y; 10 μM, R&D Systems) was added for the first 2 days of culture. Cells were cultured at 37° C. with 5% CO₂, and cell culture medium was changed every other day. After 6 days of culture, crypt organoids were isolated from Matrigel by mechanical dissociation. Isolated organoids were resuspended in TrypLE Express (Life Tech) to dissociate into single cells, then re-plated in Matrigel with ENR+CV+Y media for 2 days. Cells were once again passaged, either into freezing media (Life Tech) for cryopreservation or re-plated at approximately 200 organoids per well (24-well plate) for ISC-enriched organoid expansion. ISC-enriched organoids were passaged or differentiated every 6 days in the ENR+CV condition. To differentiate, cells were passaged as previously described, and crypt culture medium containing growth factors ENR only or ENR+CD (DAPT, 10 μM; Sigma-Aldrich) was added to each well.

In some embodiments, a method for generating and culturing human organoid is provided herein. The method contains specifically modifications based on report of Fujji M. et al. (Fujii M, Matano M, Toshimitsu K, Takano A, Mikami Y, Nishikori S, Sugimoto S, Sato T. Human Intestinal Organoids Maintain Self-Renewal Capacity and Cellular 6 Diversity in Niche-Inspired Culture Condition. Cell Stem Cell 2018, 23:787-793.e6). The specific modifications include, but are not limited to, the use of specific culture medium for human organoids. The specific culture medium disclosed herein comprises the defined “small intestinal crypt culture medium” plus components comprising EGF (Pt 2 days), Y-27632 (Pt 2 days), IGF-1, FGF-2, recombinant R-spondin 3, recombinant Noggin (or DMH-1), Afamin-Wnt3A conditioned media (1:10) (or Ly2090314), Gastrin peptide, and A83-01.

### Example 2. Guided Chemical Induction of Wnt and Inhibition of Notch Drives Paneth Cell Marker Enrichment

This example describes the methods of guided induction of Wnt and inhibition of Notch in intestinal stem cells (ISC) that drive Paneth cell marker enrichment.

Beginning with an LGR5+ISC-enriched population (ENR+CV), it was sought to profile how the modulation of Wnt and Notch signaling through small molecule inhibitors would alter the in vitro PC state, as suggested by the transcriptional profiling. Chemical induction (CI) was performed using the previously identified compounds C to drive Wnt signaling and DAPT (D), a gamma-secretase inhibitor, to inhibit Notch (ENR+CD) and bulk gene expression of Paneth cells (PC) (Lyz1, Defa1, Mmp7) and ISC (Lgr5) markers were measured every 2 days for a total of 6 days. ENR+CD-treated cells had statistically significant increases in Lyz1 (adj. p=0.005) and Mmp7 (adj. p=0.005) within 2 days compared to ENR, with differences plateauing around 4 days. Defa1 (adj. p=0.004) expression was significantly increased by day 4 and plateaued by day 6 in ENR+CD versus ENR populations. Lgr5 expression in ENR+CD at 2 days versus ENR showed an insignificant plateau of expression, which trended down by 6 days. Without being bound by theory, this can be indicative of an expansion in ‘label-retaining’ secretory precursors. The precursor population ENR+CV had no significant difference in PC or ISC markers relative to ENR. The significant increase in PC gene expression in ENR+CD relative to ENR and ENR+CV over the 6-day treatment suggests rapid enrichment following CI, supporting the hypothesis that alterations in Wnt and Notch result in superior PC enrichment in vitro. To phenotypically describe PC enrichment following CI, performed imaging and immunocytochemistry for PC-associated features was performed. After 6 days of ENR+CD, cell populations exhibited darkened annular morphology consistent with increased numbers of granule-rich cells. Confocal microscopy of whole cell clusters stained for anti-DEFA and anti-LYZ showed an increase in LYZ+ and DEFA+ cells in ENR+CD compared to both ENR and ENR+CV. Single-cell counting of confocal imaging showed a significant increase of DEFA and LYZ co-staining cells in ENR+CD (20-30% of cells) versus either ENR or ENR+CV (both <5%; adj. p=0.0001). Additionally, normalized z-axis profiles of individual co-staining cells within cell clusters revealed a consistent distribution of DEFA (luminally polarized) and LYZ (diffuse). High-resolution fluorescent imaging of individual co-staining cells from freshly isolated small intestinal crypts (in vivo equivalent) and 6-day ENR+CD-treated cells showed a similar polarized distribution of LYZ- and DEFA-stained granules, although freshly isolated cells appeared to be more granular than CI-PCs.

To confirm the extent of enrichment seen in whole population imaging, the prevalence of PCs in ENR+CD relative to ENR was assessed by flow cytometry over the course of 12 days, a longer term culture than typical for conventional organoids. An in vivo PC phenotype was identified as CD24 and LYZ co-positive cells, as per previous reports, and noted the presence of single-positive LYZ+ or single-positive CD24+ populations, indicative of alternative cell differentiation, immature, or non-physiological PCs. ENR+CD had substantial enrichment at all time points for double-positive, and single-positive LYZ+ or CD24+ populations relative to ENR, as well as a consistent decrease in the double negative population in agreement with the PC phenotype. Notably, both ENR and ENR+CD experience declines in total cell viability, with ENR+CD having greater survival at longer times, suggesting both a reduction in anoikis, a potentially physiological ‘long-lived’ PC phenotype in ENR+CD versus ENR, or an enhancement

in niche-supporting functionality. Overall, imaging and flow cytometry demonstrate a significant increase in cells morphologically resembling in vivo PCs with respect to granularity, polarity, and antimicrobial co-expression in ENR+CD compared to conventional ENR organoids.

To relate the organoid-derived PC state to in vivo PCs, an unbiased reference in vivo scRNA-seq data set was first generated. Massively parallel scRNA-seq was performed using the recently developed Seq-Well platform on epithelial cells from the ileal region of the small intestine. Quality metrics for the number of genes was measured using unique molecular identifiers (UMIs), mitochondrial genes, and ribosomal genes, all of which fell within expectations (all cells average: 1043 genes, 2168 UMIs, 5.4% ribosomal genes, 10.4% mitochondrial genes).

### Example 3. Methods of Parallel Screening Therapeutic Agents for Guided Stem Cell Differentiation

This example describes methods used for parallel screening a plurality of test compounds for therapeutic application in driving guided stem cell differentiation.

The ISC-enriched organoids were generated as described in Example 1 and maintained in ENR+CV culture medium in 24-well plates. A pool of test compounds were added to each well. Each pool contained 15 compounds that were selected based on the scaled score of their biological connectivity. The scaled score of biological connectivity was calculated based on the biological connectivity of compounds and adjusted for their chemical similarities using Tanimoto coefficient. A pool of test compounds was optimized to have a smallest possible scaled score of biological connectivity, so that there was no or minimal interactions between or among the test compounds in such a pool. As a result, the biological effect and mode of action (MOA) of each test compound in a pool could be deciphered based on the transcriptomic profile generated using massive parallel single-cell RNA-sequencing on the organoid subjected to the treatment. It will be appreciated that the mode of action refers to the specific biochemical interaction through with a compound (such as a pharmaceutical agent) produces its pharmacological effect. This can include the specific receptor(s) in which the compound interacts, signaling pathways it stimulates, inhibitors that bind it, how the compound is metabolized, active metabolites of the compound, etc. The transcriptomic profile of each organoid treated with a pool of test compounds is also used to query the Connectivity Map (CMap) developed by the Broad Institute (https://clue.io) for molecular characterization of the test compounds.

The ability of a test compound to drive differentiation of an ISC-enriched organoid to a Paneth cell-enriched organoid is evaluated based on the changes in transcriptomic profile and cell morphology as well as functional assays as described in Example 2.

### Example 4. Optimization of the Composition of Test Compounds in a Pool

This example describes the methods and uses thereof for optimizing the composition of test compounds in a pool for high-throughput, parallel perturbation-based drug screening.

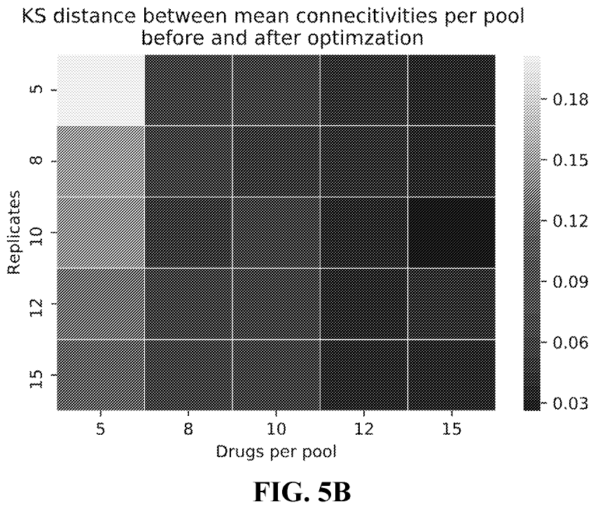

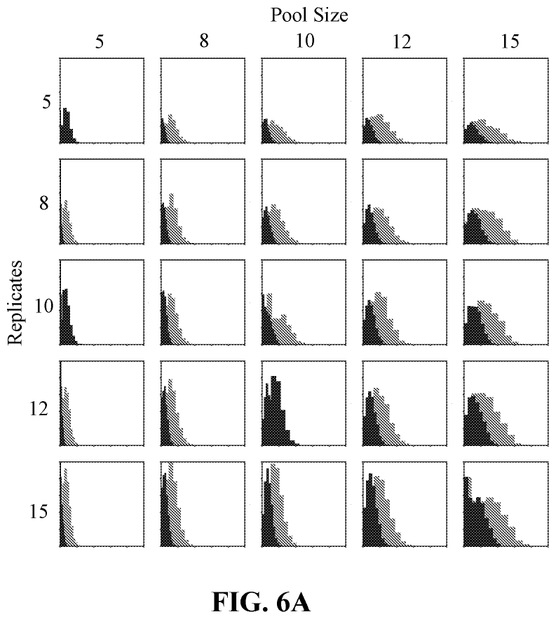

The optimization is accomplished using simulated annealing (SA) algorithm with modifications. The SA algorithm allows one to choose a subset (pool) of test compounds from a large set of test compounds, with a goal that the subset (pool) of test pools of test compounds were successfully optimized from a compound library. FIG. 4 shows that, with 50,000 iterations of optimization processes, the energy (cost) per drug in a pool is substantially reduced. The reduction in energy (cost) per drug is independent of the number of test compounds in a pool as well as the number of replicates for a test compound. Because the structure similarity of compounds in a library is usually already optimized to be minimal, i.e. most of the compounds in a library have distinct structures, the optimization process employed in the present invention does not substantially reduce the mean similarity per pool (FIGS. 5A-5B). Surprisingly, however, the optimization process employed in the present invention substantially reduces the mean connectivity per pool, and the reduction in mean connectivity per pool is independent of the number of test compounds in a pool as well as the number of replicates for a test compound (FIGS. 6A-6B). Another characteristic of the methods disclosed herein is that the repeated compound pairing is rare. The frequency of repeated compound paring does not change substantially as the number of replicates increases. However, understandably, as the number of test compounds in a pool increases, the frequency (chance) of repeated compound paring increases (FIG. 7).

### Example 5. Optimization of the Rational Design of Pools to Maximize Perturbations Using Minimal Biomass

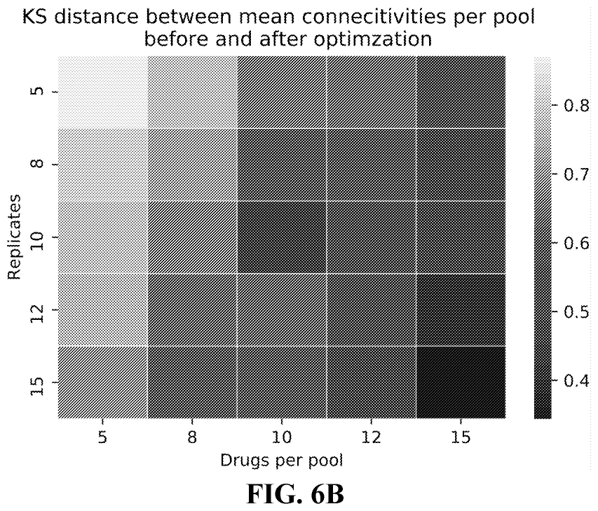

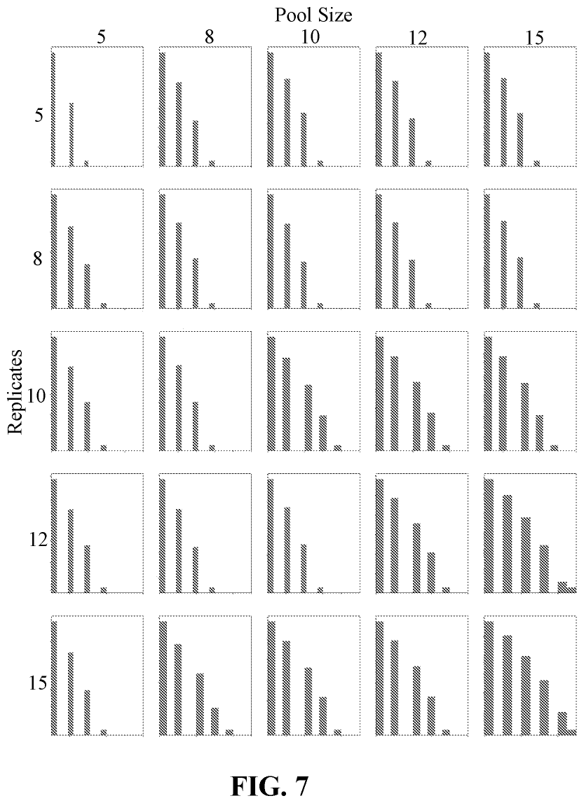

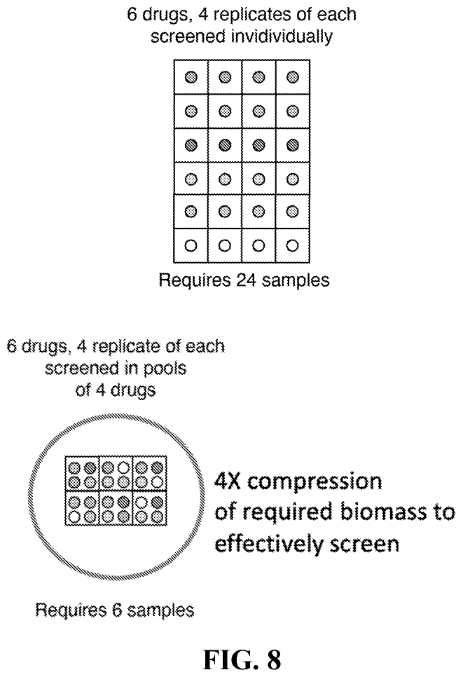

Success in identifying and developing agents with specific effects on cells, tissue, organs, organ systems, and the like needs better systems and models for identifying agents with specific effects. This is particularly challenging when the effect(s) needs to be discerned in biomass limited samples as previously discussed. As shown in FIG. 8, pooling of agents can facilitate a compression of the required biomass. However, randomized pools of agents such as in conventional methods fall short of due to inherent trade-offs as shown in FIG. 10. As shown in FIG. 9, computational methods, such as those described in several embodiments and Examples herein, can facilitate the rational design of agent pools for compressed screening that can maximize the perturbations and thus minimize the amount of biomass needed while reducing the sacrifices that must be made when using traditional randomized pools (see e.g. FIG. 10).

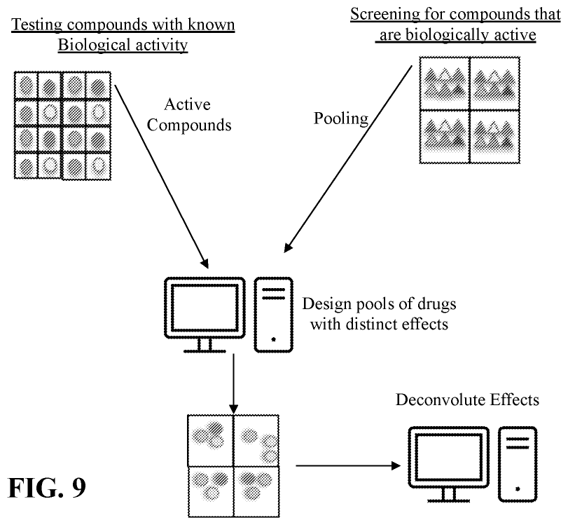

Optimizing independent agent pools for compressed screening coupled with decoding pooled screens can reduce the overlap of agent similarity and provide many-fold compression not achievable by randomized pools. FIG. 11 can demonstrate that optimization independent pools coupled with decoding pooled screens can, among other things, reduce the number or replicates per drug needed for an effective screen. In some embodiments, a matrix of drug-drug similarity can be used to calculate the ‘cost’ or ‘energy’ of any given pool and optimize for minimally interacting pools in bio-active libraries, which can provide for many-fold compression. In traditional screening, including randomized pools, the assumption is that generally each agent will elicit an effect and/or that there are synergies between agents in a pool, whose effect will be apparent. The compression and optimization methods herein can allow for an approach that is the inverse of looking for synergies and does not necessarily rely on the assumption that the agent have any effect on the biomass.

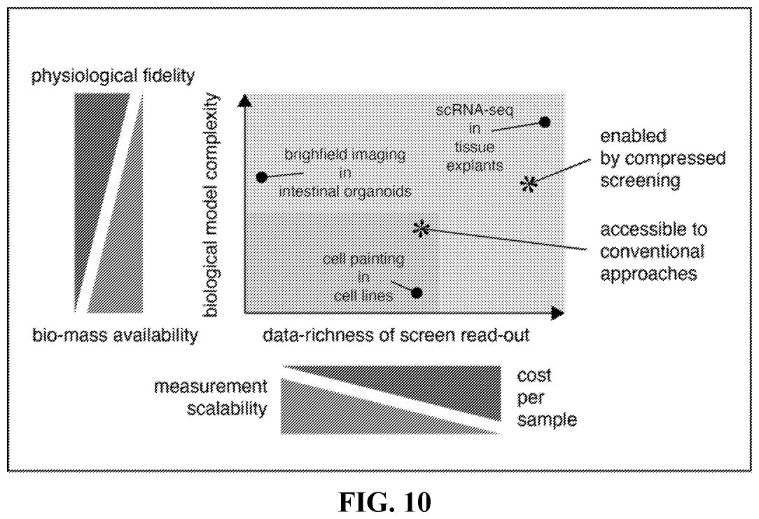

Pooled screens can be decoded by deconvoluting the output. FIG. 12 shows a general linear model for deconvolution of pooled results to determine individual agent (e.g. compound, drug, or other potentially active agent) effect where the model can be solved for the effect of the agent on the measured parameter. The parameters dictate screen “hits”.

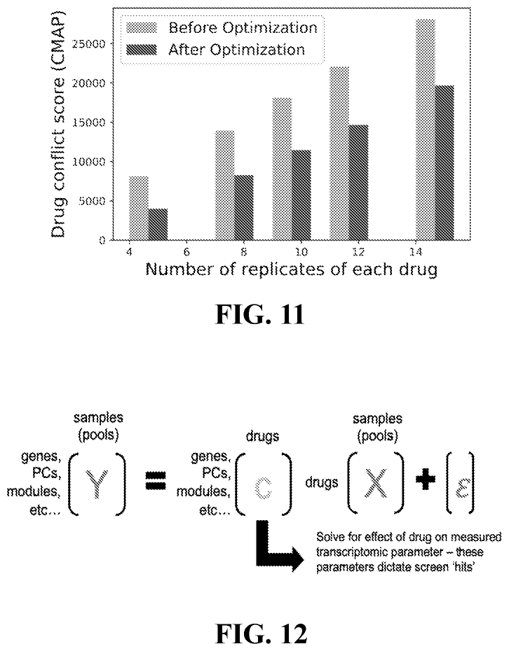

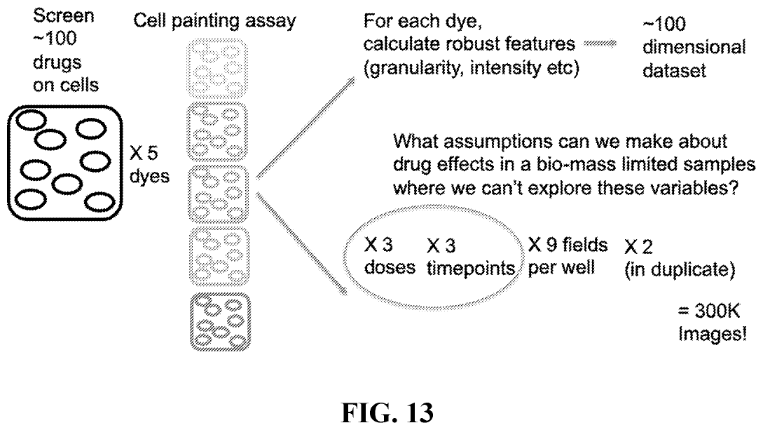

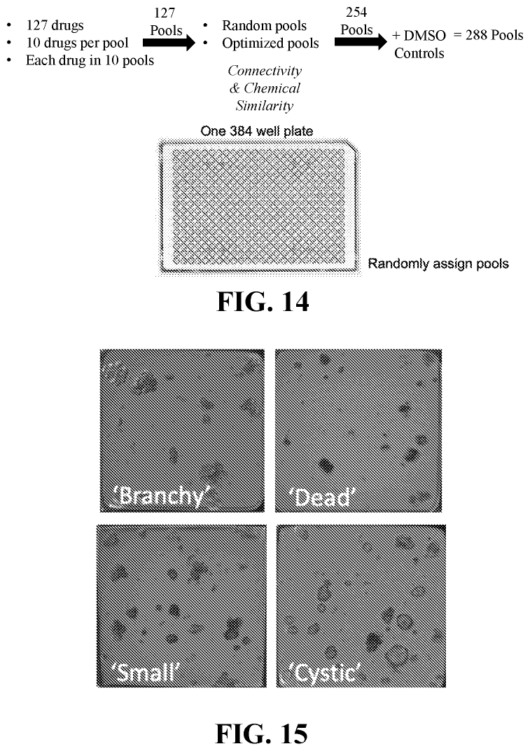

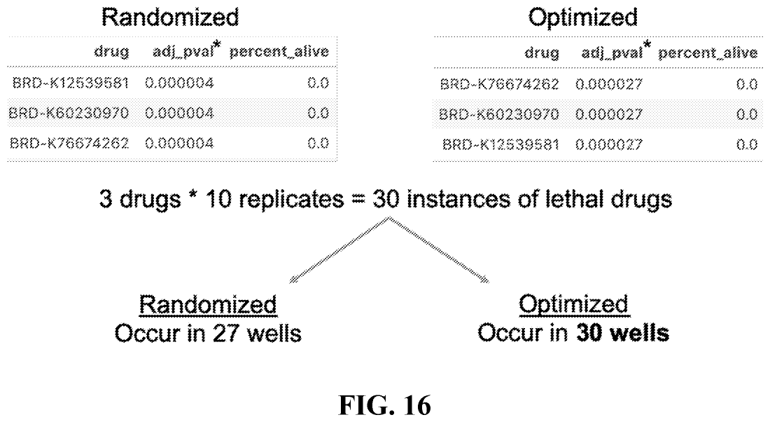

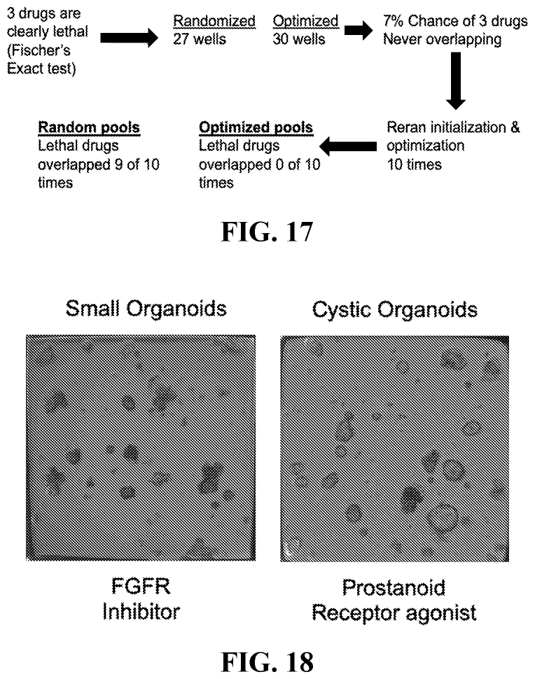

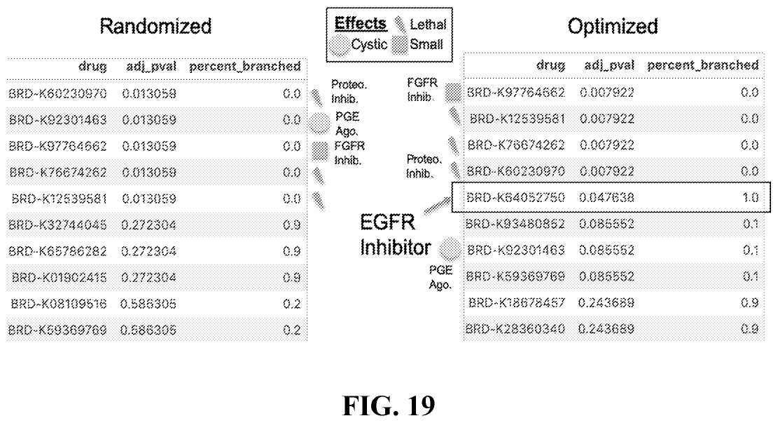

FIG. 13 shows an exemplary design of a data set for a compressed screening assay. A study was completed using an exemplary complex sample: human intestinal organoids. FIG. 14 shows an experimental design of a compressed screening assay to compare results from randomized pools versus optimized, rationally-designed pools. A first pass analysis of a simple readout was applied as shown in FIG. 15. Cell morphology of the human intestinal organoids was examined. All images were observed and were classified into one of four different classes: “branchy”, “dead”, “small”, and “cystic”. FIG. 16 show that results from the simple readout demonstrated that three drugs are lethal, which were identified in 27 wells in a conventional randomized screen and 30 wells of an optimized screen described by one or more embodiments herein. FIG. 17 can demonstrate that optimization of the rational design of pools in the compressed screening can reduce the redundancy in samples needed to demonstrate an effect of an agent in a screen. FIG. 18 can demonstrate that some phenotypes do not need optimization to discern agent effect(s). For example, in human intestinal organoids that displayed a “small” or “cystic” phenotype as determined during a first pass analysis (see e.g. FIG. 15) both randomized and optimized pools identified one agent driving each phenotype (FGFR inhibitor in the case of the “small” phenotype and prostanoid receptor agonist in the case of the “cystic” phenotype).

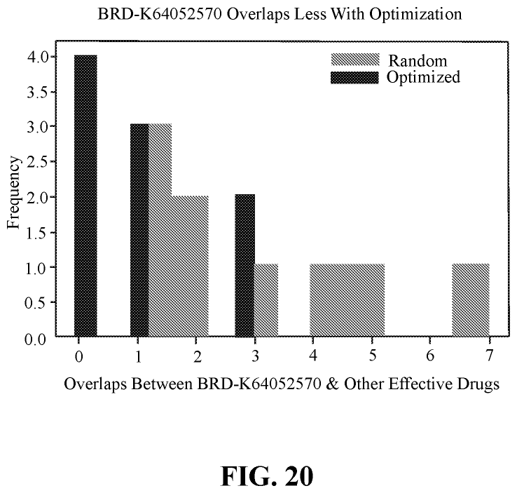

FIG. 19 can demonstrate that some phenotypes benefit from optimization to discern agent effect(s). For example, in human intestinal organoids that displayed a “branched” phenotype optimization provided clearer insight into agents affecting the phenotype.

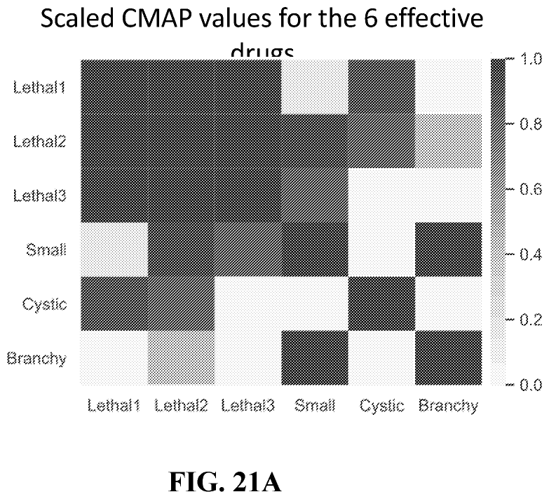

FIG. 20 can demonstrate that the effects of some agents can be buried or “swamped out” using conventional randomized screening techniques. For example, in the case of the human intestinal organoids the effect of BRD-K64052570 (see e.g. FIG. 19) could have been lost without optimization, particularly in the branched phenotype. This loss can be attributed to different agents having similar effects. As shown in FIG. 20, BRD-K64052570 overlapped with other effective drugs and optimization reduced the overlap with these drugs and allow the effect to be discerned.

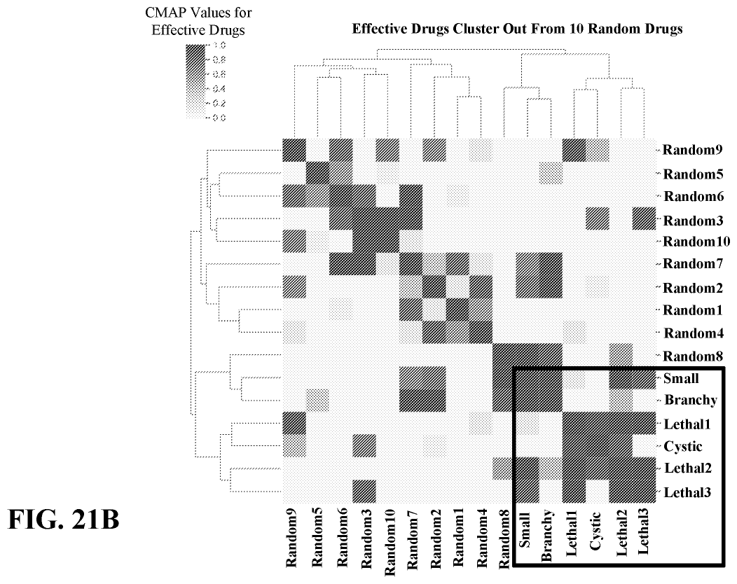

FIGS. 21A-21B can demonstrate that effective agents can be connected by CMAP scores. In the case of the human intestinal organoids the effective agents were connected by high CMAP scores.

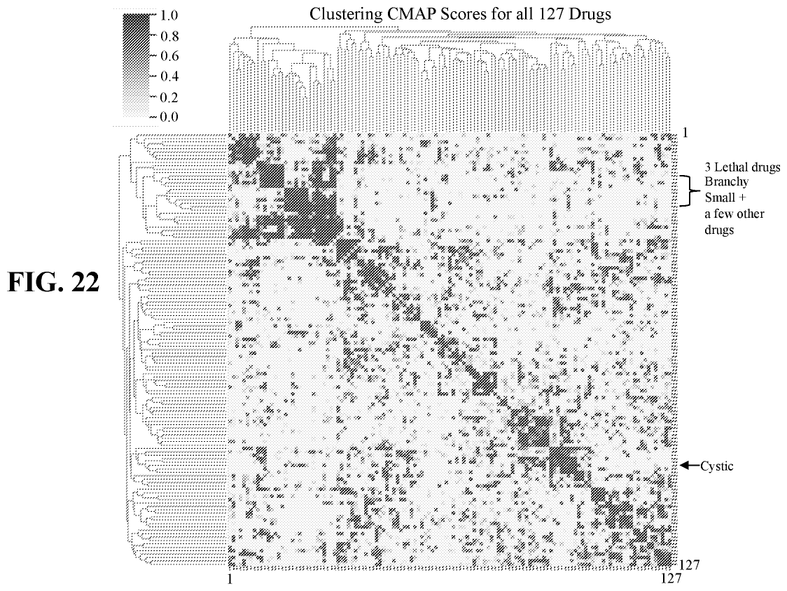

FIG. 22 can demonstrate clustering CMAP scores for all 127 agents tested against the human intestinal organoids. FIG. 22 can demonstrate that some drugs with similar effects clustered by phenotype based on effect. For example, 3 lethal drugs clustered with the “small” and “branchy” phenotype. Table 1 sets forth the drugs identified in FIG. 22. Each tick on the x and y axis identifies each drug from 1 to 127 presented in numerical order despite each number not being shown.

TABLE 1

| Drug | FIG. 22 ID |
|---|---|
| Random 78 | 1 |
| Random 66 | 2 |
| Random 86 | 3 |
| Random 118 | 4 |
| Random 60 | 5 |
| Random 45 | 6 |
| Random 84 | 7 |
| Random 96 | 8 |
| Random 117 | 9 |
| Random 36 | 10 |
| Random 98 | 11 |
| Random 100 | 12 |
| Branchy | 13 |
| Random 83 | 14 |
| Small | 15 |
| Random 82 | 16 |
| Lethal 1 | 17 |
| Lethal 3 | 18 |
| Random 62 | 19 |
| Random 57 | 20 |
| Random 121 | 21 |
| Lethal 2 | 22 |
| Random 17 | 23 |
| Random 24 | 24 |
| Random 52 | 25 |
| Random 44 | 26 |
| Random 61 | 27 |
| Random 72 | 28 |
| Random 16 | 29 |
| Random 93 | 30 |
| Random 102 | 31 |
| Random 9 | 32 |
| Random 64 | 33 |
| Random 80 | 34 |
| Random 87 | 35 |
| Random 8 | 36 |
| Random 114 | 37 |
| Random 26 | 38 |
| Random 119 | 39 |
| Random 19 | 40 |
| Random 25 | 41 |
| Random 77 | 42 |
| Random 21 | 43 |
| Random 38 | 44 |
| Random 67 | 45 |
| Random 6 | 46 |
| Random 111 | 47 |
| Random 7 | 48 |
| Random 33 | 49 |
| Random 75 | 50 |
| Random 85 | 51 |
| Random 63 | 52 |
| Random 105 | 53 |
| Random 5 | 54 |
| Random 116 | 55 |
| Random 58 | 56 |
| Random 108 | 57 |
| Random 113 | 58 |
| Random 15 | 59 |
| Random 34 | 60 |
| Random 48 | 61 |
| Random 39 | 62 |
| Random 95 | 63 |
| Random 43 | 64 |
| Random 41 | 65 |
| Random 99 | 66 |
| Random 27 | 67 |
| Random 112 | 68 |
| Random 22 | 69 |
| Random 49 | 70 |
| Random 107 | 71 |
| Random 46 | 72 |
| Random 32 | 73 |
| Random 1 | 74 |
| Random 56 | 75 |
| Random 53 | 76 |
| Random 76 | 77 |
| Random 47 | 78 |
| Random 30 | 79 |
| Random 37 | 80 |
| Random 29 | 81 |
| Random 50 | 82 |
| Random 115 | 83 |
| Random 71 | 84 |
| Random 92 | 85 |
| Random 81 | 86 |
| Random 10 | 87 |
| Random 101 | 88 |
| Random 74 | 89 |
| Random 89 | 90 |
| Random 20 | 91 |
| Random 106 | 92 |
| Random 31 | 93 |
| Random 40 | 94 |
| Random 109 | 95 |
| Random 18 | 96 |
| Random 79 | 97 |
| Cystic | 98 |
| Random 51 | 99 |
| Random 70 | 100 |
| Random 73 | 101 |
| Random 110 | 102 |
| Random 55 | 103 |
| Random 65 | 104 |
| Random 4 | 105 |
| Random 88 | 106 |
| Random 3 | 107 |
| Random 35 | 108 |
| Random 120 | 109 |
| Random 11 | 110 |
| Random 12 | 111 |
| Random 90 | 112 |
| Random 23 | 113 |
| Random 103 | 114 |
| Random 68 | 115 |
| Random 94 | 116 |
| Random 104 | 117 |
| Random 97 | 118 |
| Random 42 | 119 |
| Random 69 | 120 |
| Random 28 | 121 |
| Random 91 | 122 |
| Random 13 | 123 |
| Random 14 | 124 |
| Random 54 | 125 |
| Random 2 | 126 |
| Random 59 | 127 |

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A computer-implemented method for selecting a subset of test compounds from a group of test compounds to place in a pool of test compounds, wherein the pool of test compounds are to be evaluated in parallel for their biological functions, comprising, by one or more computing devices:
a. receiving a request to evaluate a group of test compounds;
b. determining chemical similarity of each test compound with every other test compound in the group of test compounds;
c. determining a biological connectivity of each test compound with every other test compound in the group of test compounds, wherein the biological connectivity is assessed based on a transcriptional profile, mode of action, gene targets, effect on protein-protein interactions, or any combination thereof of each test compound;
d. calculating an optimization score based in part on the chemical similarity and the biological connectivity of each test compound in the group; and
e. based on the optimization score, assigning test compounds into the subset that can be evaluated together with minimal interaction or interference with other test compounds in a given subset.

2. The computer-implemented method of claim 1, wherein assigning test compounds into the subset is based on a gradient-free optimization algorithm.

3. The computer-implemented method of claim 1, further comprising selecting a plurality of subsets such that each test compound is placed into at least one subset and a lowest total energy is determined for the plurality of subsets.

4. The computer-implemented method of claim 1, further comprising:
f. receiving a number of test compounds to include in a plurality of subgroups of test compounds;
g. determining the chemical similarity and the biological connectivity of each of the plurality of subgroups to determine the energy of each subgroup; and
h. based on the determined energy for each subgroup, selecting subsets of test compounds that minimize the determined energy for each subgroup.

5. The computer-implemented method of claim 1, further comprising:
f. calculating a scaled score of biological connectivity, a scaled score of chemical similarity, or both for each pair of test compounds evaluated, wherein the scaled scores of biological connectivity and chemical similarity are based on a scaling function; and
g. based on the scaled score of biological connectivity, the scaled score of chemical similarity, or both for each pair of test compounds evaluated, selecting a subset of the group of test compounds to place in a pool for evaluation.

6. The computer-implemented method of claim 1, wherein the biological connectivity is based on a connectivity map of common gene expression signatures, and wherein the chemical similarity is based on the calculation of Tamonoto coefficient for each pair of test compounds.

7. The computer-implemented method of claim 1, wherein the pools of test compounds are optimized to test for combinatorial effects of agents versus isolation of individual agent effects.

8. A method of parallel screening for therapeutic agents for treating a disease, the method comprising:
a. contacting a cell or a tissue with a pool of test compounds in parallel, wherein the test compounds in the pool are selected using the methods of any one of claims 1-7;
b. evaluating the effect of the test compounds on the cell or tissue; and
c. selecting one or more test compounds with a desired activity,
whereby a plurality of test compounds are screened in parallel for therapeutic application in treating the disease.

9. The method of claim 8, wherein the tissue is an in vitro cultured organoid.

10. The method of claim 9, wherein the organoid is derived from stem cells originated from tissues or organs comprising the intestine, liver, pancreas, brain, ovary, uterus, skin, esophagus, lung, spleen, kidney, bone, bone marrow, brain, central nervous system, peripheral nervous system, blood, cartilage, fat, and eye.

11. The method of claim 8, wherein the number of test compounds in a pool is any number between 1 and 1,000,000,000,000.

12. The method of claim 8, wherein the effect of the test compounds is evaluated by measuring changes in biological activities comprising transcriptomics, genomics, epigenomics, proteomics, genetics, epigenetics, metabolomics, multiomics, phenotype, or any combination thereof.

13. The method of any of claim 8, wherein the effect of the test compounds is evaluated by measuring changes in transcriptomics.

14. The method of claim 12, wherein the measurement of the effect of the test compounds is performed at single-cell level.

15. A method of screening for therapeutic agents for guiding cell differentiation, the method comprising:
a. contacting an in vitro cultured organoid or a cultured cell with a pool of test compounds in parallel, wherein the test compounds in the pool are selected using the methods of any one of claims 1-7;
b. evaluating the effect of test compounds on cell differentiation of the organoid or cultured cell; and
c. selecting the one or more test compounds capable of guiding the differentiation of the organoid or the cultured cell from a first cell state to a second cell state.

16. The method of claim 15, wherein the cultured cell is a stem cell.

17. The method of claim 15, wherein the organoid is derived from stem cells that originated from, were isolated from, or were derived from tissues or organs comprising the intestine, liver, pancreas, brain, ovary, uterus, skin, esophagus, lung, spleen, kidney, bone, bone marrow, brain, central nervous system, peripheral nervous system, blood, cartilage, fat, and eye.

18. The method of claim 15, wherein the cultured cell is isolated from tissues or organs comprising the intestine, liver, pancreas, brain, ovary, uterus, skin, esophagus, lung, spleen, kidney, bone, bone marrow, brain, central nervous system, peripheral nervous system, blood, cartilage, fat, and eye.

19. The method of claim 15, wherein the number of test compounds in a pool is any number between 1 and 1,000,000.

20. The method of claim 15, wherein the differentiation of the organoid or cultured cell is measured by changes in comprising phenotype, genotype, genomics, epigenomics, transcriptomics, genetics, epigenetics, proteomics, multiomics, metabolomics, or any combination thereof.

21. The method of claim 20, wherein the measurement is performed at single-cell level.

22. The method of claim 15, wherein the second cell state is a differentiated, functional cell selected from the group consisting of: a Paneth cell, goblet cell, M cell, Tuft cell, enteroendocrine cell, enterocytes, hepatocyte, pancreatic B-cell, pancreatic alpha-cell, neuron, glia cell, brain cell, keratinocyte, melanocyte, epithelial cell, endothelial cell, hematopoietic cell, T lymphocyte, B lymphocyte, natural killer cell, dendritic cell, macrophage, monocyte, neutrophil, eosinophil, basophil, megakaryocyte, platelet, adipocyte, osteoblast, osteoclast, chondrocyte, and a combination thereof.

23. A method for determining specific biological effect, pharmacological effect, or both of a test compound in a test pool, comprising:
a. forming an optimized pool containing the test compound of interest according to any one claims 1-7;
b. testing the biological and/or pharmacological effects of the pool of testing compounds according to any of the preceding claims; and
c. performing deconvolution of the biological effects, pharmacological effects, or both of the pool, wherein the deconvolution comprises computational de-coding using methods comprising large linear computational models,
whereby the specific biological effect, pharmaceutical effect, or both of a test compound are determined.

24. A method of determining one or more characteristics of a disease, comprising:
contacting a biological sample in vitro with a pool of test compounds in parallel, wherein the test compounds in the pool are selected using the methods of any one of claims 1-7;
evaluating the effect of one or more of the test compounds in the pool of test compounds on the biological sample,
whereby the specific biological effect, pharmaceutical effect, or both of a test compound are determined and whereby the biological effect, pharmaceutical effect, or both of a test compound determined is/are indicative of one or more disease characteristics.

25. The method of claim 24, wherein the one or more disease characteristics is evaluated by measuring or evaluating one or more of the following: expression, activity, or function of one or more genes, proteins, gene programs, biological pathways, cell processes, cell or tissue functions, or combinations thereof in the biological sample.

26. The method of claim 24, wherein evaluating the effect of one or more of the test compounds comprises measuring changes in one or more biologic activities of the biological sample.

27. The method of claim 26, wherein measuring changes in one or more biologic activities comprises a transcript or transcriptome analysis, a gene or genomic analysis, an epigenome analysis, a protein or proteome analysis, a metabolomic analysis, a multiomic analysis, a phenotype analysis, a genetic analysis, or a combination thereof.

28. The method of claim 24, wherein evaluating the effect of one or more of the test compounds further comprises performing deconvolution of the biological effects, pharmacological effects, or both the pool, wherein the deconvolution comprises computational de-coding using one or more methods comprising large linear computational models.

29. The method of claim 24, wherein the biological sample is a cell or cell population, organoid, or tissue.

30. The method of claim 24, wherein the biological sample is a biopsy sample obtained from a subject.

31. The method of claim 24, wherein the biological sample is isolated from, derived from, or comprises, cells or tissue of the intestine, liver, pancreas, ovary, uterus, skin, esophagus, lung, spleen, kidney, bone, bone marrow, brain, central nervous system, peripheral nervous system, blood, cartilage, fat, eye, heart, lymph node, lymphatic system, thyroid, endocrine system tissue or gland, or a combination thereof.

32. The method of claim 24, wherein the test pool is an optimized test pool.

33. The method of claim 24, wherein the number of test compounds in the test pool is any number from 1 to 1,000,000.

34. A method of diagnosing, prognosing, monitoring, or staging a disease in a subject, comprising:
contacting, in vitro, a biological sample obtained from the subject with a pool of test compounds in parallel, wherein the test compounds in the pool are selected using the methods of any one of claims 1-7;
evaluating the effect of one or more of the test compounds in the pool of test compounds on the biological sample,
whereby the specific biological effects, pharmaceutical effects, or both are determined and whereby the specific biological effects, pharmaceutical effects, or both of a test compound determined is/are indicative of a disease, a disease symptom, and/or stage of a disease in the subject.

35. The method of claim 34, wherein evaluating the effect of one or more of the test compounds comprises measuring changes in one or more biologic activities of the biological sample.

36. The method of claim 35, wherein measuring changes in one or more biologic activities comprises a transcript or transcriptome analysis, a gene or genomic analysis, an epigenome analysis, a protein or proteome analysis, a multiomic analysis, a metabolomic analysis, a phenotype analysis, a genetic analysis, or a combination thereof.

37. The method of claim 34, wherein evaluating the effect of one or more of the test compounds further comprises performing deconvolution of the biological effects, pharmacological effects, or both of the pool, wherein the deconvolution comprises computational de-coding using one or more methods comprising large linear computational models.

38. The method of claim 34, wherein the biological sample is a cell or cell population, organoid, or tissue.

39. The method of claim 34, wherein the biological sample is a biopsy sample obtained from a subject.

40. The method of claim 34, wherein the biological sample is isolated from, derived from, or comprises cells or tissue of the intestine, liver, pancreas, ovary, uterus, skin, esophagus, lung, spleen, kidney, bone, bone marrow, brain, central nervous system, peripheral nervous system, blood, cartilage, fat, eye, heart, lymph node, lymphatic system, thyroid, endocrine system tissue or gland, or a combination thereof.

41. The method of claim 34, wherein the test pool is an optimized test pool.

42. The method of claim 34, wherein the number of test compounds in the test pool is any number from 1 to 1,000,000.

43. A pharmaceutical formulation comprising:
one or more active agents; and
a pharmaceutically acceptable carrier,
wherein the one or more active agents is identified as an active agent by performing a method as in any one of claims 8-33.

44. A method of guiding differentiation of a cell from a first cell state to a second cell state comprising:
contacting a cell or cell population with one or more compounds capable of guiding differentiation of a cell, wherein the one or more active agents is identified as an active agent by performing a method as in any one of claims 15-22.

45. The method of claim 44, wherein the cell or cell population is a stem cell or stem cell population.

46. A differentiated cell, cell population, tissue, or organoid, wherein the differentiated cell, cell population, tissue, or organoid is produced by a method of guided differentiation as in any one of claims 44-45.

47. A method of treating a subject in need thereof, comprising:
administering a pharmaceutical formulation as in claim 44, a differentiated cell, cell population, tissue, or organoid as in claim 46, or both to the subject in need thereof.
